(19) United States
(12) Patent Application Publication
(10) Pub. No.: US 2018/0162821 A1
(43) Pub. Date: Jun. 14, 2018

(54) FACTOR XIA MACROCYCLES WITH NOVEL P1 GROUPS

(71) Applicant: BRISTOL-MYERS SQUIBB COMPANY, Princeton, NJ (US)

(72) Inventors: James R. Corte, Lawrenceville, NJ (US); Andrew K. Dilger, Ewing, NJ (US); William R. Ewing, Yardley, PA (US); Donald J.P. Pinto, Churchville, PA (US); Wu Yang, Princeton Junction, NJ (US)

(21) Appl. No.: 15/837,034

(22) Filed: Dec. 11, 2017

Related U.S. Application Data
(60) Provisional application No. 62/433,969, filed on Dec. 14, 2016.

(51) Int. Cl. C07D 225/06; C07D 403/04; C07D 401/04; C07D 471/04; C07D 471/08; C07D 487/08; C07D 245/06; A61P 7/02

(52) U.S. Cl. CPC C07D 225/06; C07D 403/04; C07D 401/04; A61P 7/02; C07D 471/08; C07D 487/08; C07D 245/06; C07D 471/04

(57) ABSTRACT

The present invention provides compounds of Formula (I):

P-Ma (I)

or stereoisomers, tautomers, or pharmaceutically acceptable salts thereof, wherein all the variables are as defined herein. These compounds are selective factor XIa inhibitors or dual inhibitors of FXIa and plasma kallikrein. This invention also relates to pharmaceutical compositions comprising these compounds and methods of treating thromboembolic and/or inflammatory disorders using the same.

### CROSS REFERENCE TO RELATED APPLICATIONS

This application is entitled to priority pursuant to 35 U.S.C. § 119(e) to U.S. provisional patent application No. 62/433,969, filed on Dec. 14, 2016, which is incorporated herein in its entirety.

### FIELD OF THE INVENTION

The present invention relates generally to novel macrocyclic compounds, and their analogues thereof, which are inhibitors of factor XIa and/or plasma kallikrein, compositions containing them, and methods of using them, for example, for the treatment or prophylaxis of thromboembolic disorders, or for the treatment of retinal vascular permeability associated with diabetic retinopathy and diabetic macular edema.

### BACKGROUND OF THE INVENTION

Thromboembolic diseases remain the leading cause of death in developed countries despite the availability of anticoagulants such as warfarin (COUMADIN®), heparin, low molecular weight heparins (LMWH), and synthetic pentasaccharides and antiplatelet aunts such as aspirin and clopidogrel (PLAVIX®). The oral anticoagulant warfarin, inhibits the post-translational maturation of coagulation factors VII, IX, X and prothrombin, and has proven effective in both venous and arterial thrombosis. However, its usage is limited due to its narrow therapeutic index, slow onset of therapeutic effect, numerous dietary and drug interactions, and a need for monitoring and dose adjustment. Thus discovering and developing safe and efficacious oral anticoagulants for the prevention and treatment of a wide range of thromboembolic disorders has become increasingly important.

One approach is to inhibit thrombin generation by targeting the inhibition of coagulation factor XIa (FXIa). Factor XIa is a plasma serine protease involved in the regulation of blood coagulation, which is initiated in vivo by the binding of tissue factor (TF) to factor VII (FVII) to generate factor VIIa (FVIIa). The resulting TF:FVIIa complex activates factor IX (FIX) and factor X (FX) that leads to the production of factor Xa (FXa). The generated FXa catalyzes the transformation of prothrombin into small amounts of thrombin before this pathway is shut down by tissue factor pathway inhibitor (TFPI). The process of coagulation is then further propagated via the feedback activation of Factors V, VIII and XI by catalytic amounts of thrombin. (Gailani, D. et al., Arterioscler. Thromb. Vase. Biol., 27:2507-2513 (2007).) The resulting burst of thrombin converts fibrinogen to fibrin that polymerizes to form the structural framework of a blood clot, and activates platelets, which are a key cellular component of coagulation (Hoffman, M., Blood Reviews, 17:S1-S5 (2003)). Therefore, factor XIa plays a key role in propagating this amplification loop and is thus an attractive target for anti-thrombotic therapy.

Plasma prekallikrein is a zymogen of a trypsin-like swine protease and is present in plasma at 35 to 50 μg/mL. The gene structure is similar to that of factor XI. Overall, the amino acid sequence of plasma kallikrein has 58% homology to factor XI. Plasma kallikrein is thought to play a role in a number of inflammatory disorders. The major inhibitor of plasma kallikrein is the serpin C₁ esterase inhibitor. Patients who present with a genetic deficiency in C1 esterase inhibitor suffer from hereditary angioedema (HAE) which results in intermittent swelling of face, hands, throat, gastro-intestinal tract and genitals. Blisters formed during acute episodes contain high levels of plasma kallikrein which cleaves high molecular weight kininogen liberating bradykinin leading to increased vascular permeability. Treatment with a large protein plasma kallikrein inhibitor has been shown to effectively treat HAE by preventing the release of bradykinin which causes increased vascular permeability (A. Lehmann “Ecallantide (DX-88), a plasma kallikrein inhibitor for the treatment of hereditary angioedema and the prevention of blood loss in on-pump cardiothoracic surgery” Expert Opin. Biol. Ther. 8, p1187-99).

The plasma kallikrein-kinin system is abnormally abundant in patients with advanced diabetic macular edema. It has been recently published that plasma kallikrein contributes to retinal vascular dysfunctions in diabetic rats (A. Clermont et al. “Plasma kallikrein mediates retinal vascular dysfunction and induces retinal thickening in diabetic rats” Diabetes, 2011, 60, p1590-98). Furthermore, administration of the plasma kallikrein inhibitor ASP-440 ameliorated both retinal vascular permeability and retinal blood flow abnormalities in diabetic rats. Therefore, a plasma kallikrein inhibitor should have utility as a treatment to reduce retinal vascular permeability associated with diabetic retinopathy and diabetic macular edema. Other complications of diabetes such as cerebral hemorrhage, nephropathy, cardiomyopathy and neuropathy, all of which have associations with plasma kallikrein may also be considered as targets for a plasma kallikrein inhibitor.

To date, no small molecule synthetic plasma kallikrein inhibitor has been approved for medical use. The large protein plasma kallikrein inhibitors present risks of anaphylactic reactions, as has been reported for Ecallantide. Thus there remains a need for compounds that inhibit plasma kallikrein, that do not induce anaphylaxis and that are orally available. Furthermore, the molecules in the known art feature a highly polar and ionizable guanidine or amidine functionality. It is well known that such functionalities may be limiting to gut permeability and therefore to oral availability.

### SUMMARY OF THE INVENTION

The present invention provides novel macrocyclic compounds, their analogues, including stereoisomers, tautomers, pharmaceutically acceptable salts, or solvates thereof, which are useful as selective inhibitors of serine protease enzymes, especially factor XIa and/or plasma kallikrein.

The present invention also provides processes and intermediates for making the compounds of the present invention.

The present invention also provides pharmaceutical compositions comprising a pharmaceutically acceptable carrier and at least one of the compounds of the present invention or stereoisomers, tautomers, pharmaceutically acceptable salts, or solvates thereof.

The compounds of the invention may be used in the treatment and/or prophylaxis of thromboembolic disorders.

The compounds of the invention may be used in the treatment of retinal vascular permeability associated with diabetic retinopathy and diabetic macular edema.

The compounds of the present invention may be used in therapy.

The compounds of the present invention may be used for the manufacture of a medicament for the treatment and/or prophylaxis of a thromboembolic disorder.

The compounds of the invention can be used alone, in combination with other compounds of the present invention, or in combination with one or more, preferably one to two other agent(s).

These and other features of the invention will be set forth in expanded form as the disclosure continues.

### DETAILED DESCRIPTION OF THE INVENTION

In a first aspect, the present disclosure provides, inter alia, a compound of Formula (I):

P-Ma (I)

or a stereoisomer, a tautomer, a pharmaceutically acceptable salt, or a solvate thereof, wherein:

Ma is independently selected from Formulae (M1), (M2), (M3), and (M4):

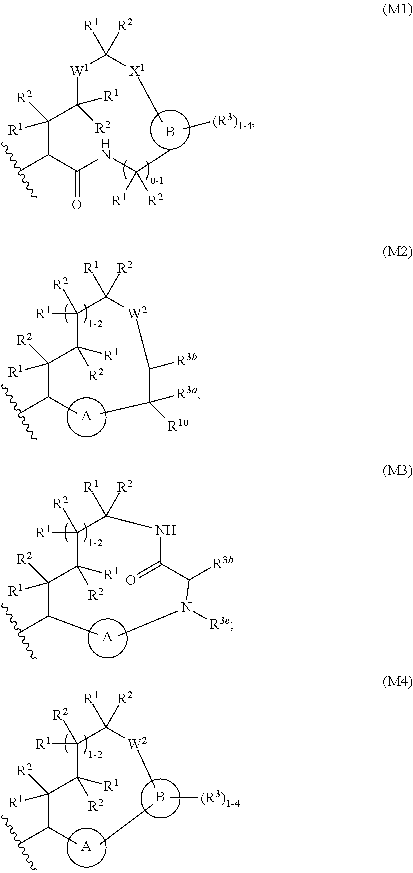

P is independently selected from Formulae (P1), (P2), (P3), (P4 ), (P5), (P6), (P7), (P8), P(9), P(10), P(11), P(12), P(13), and P(14):

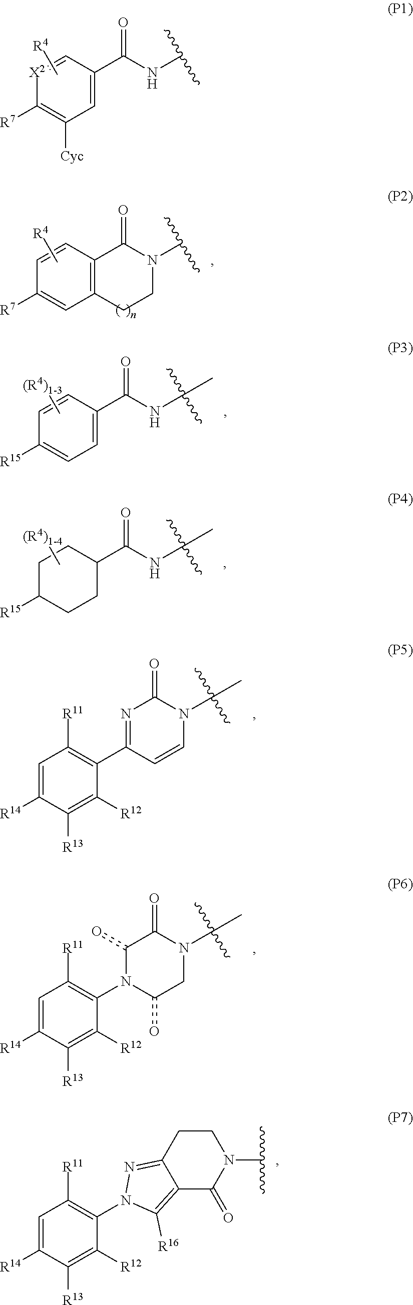

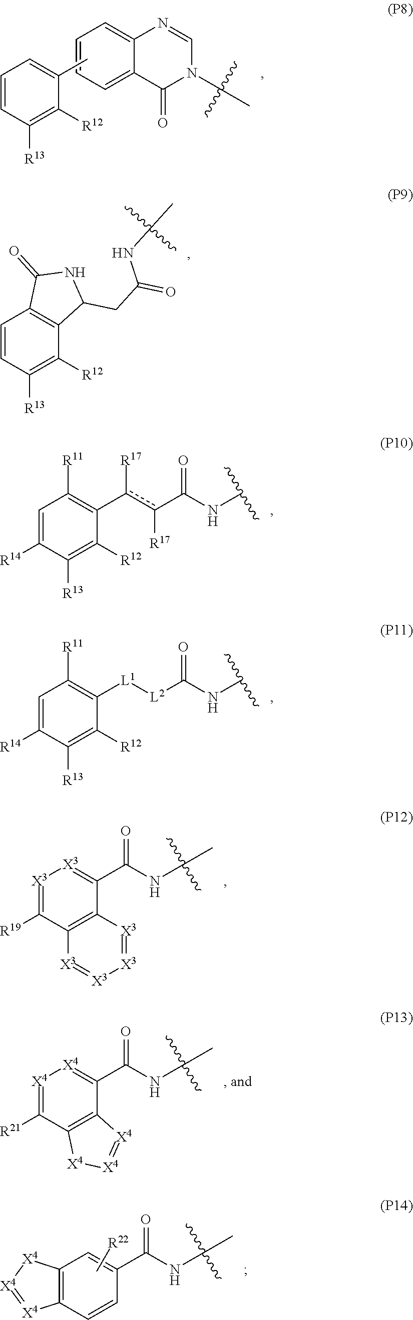

CyC is C₃₋₆ cycloalkyl;

ring A is independently selected from

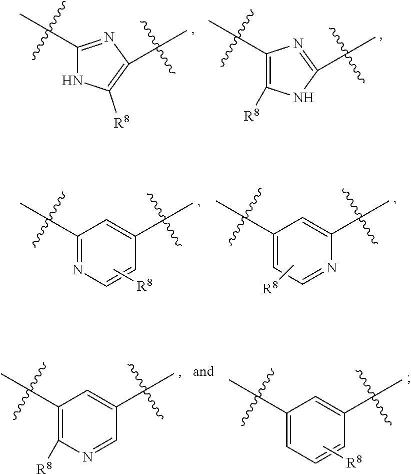

Ring B is independently selected from aryl and 5- to 10-membered heterocyclyl;

W¹ is independently selected from (CR¹R²)₁₋₂, O, NH, and N(C₁₋₄alkyl);

W² is independently selected from —C(O)NH, —NHC(O)—, and —NHC(O)CH₂—;

X¹ is independently selected from —CR²³NH—, —NHC(═O)— and —C(═O)NH—; provided when W¹ is O, NH, and N(C₁₋₄alkyl), X¹ is not —NHC(═O)—;

X² is independently selected from N and CR⁴,

X³ is independently selected from CR¹⁸ and N;

X⁴ is independently selected from CR²⁰, O, S, N, NH, and NC₁₋₄alkyl;

L¹ is independently selected from CH₂, O, NH, NC₁₋₄alkyl, and S;

L² is independently selected from NH and NC₁₋₄alkyl;

R¹ and R² are independently selected from H, halogen, C₁₋₄alkyl substituted with 0-4 R^{e}, OR^{b}, and C₃₋₅ cycloalkyl substituted with 1-4 R⁶;

alternatively, two adjacent R¹ groups form a bond;

R³ is independently selected from H, NO₂, ═O, halogen, C₁₋₄ alkyl substituted with 1-5 R⁵, C₂₋₄alkenyl substituted with 1-5 R⁵, C₂₋₄ alkynyl substituted with 1-5 R⁵, CN, —(CH₂)ₙ—-OR^{b}, —(CH₂)ₙ—NR^{a}R^{a}, —(CH₂)ₙ—C(═O)R^{b}, —(CH₂)ₙ—C(═O)OR^{b}, —(CH₂)ₙ—NR^{a}C(═O)OR^{b}, —(CH₂)ₐ—NR^{a}C(═O)R^{b}, —(CH₂)ₙ—NR^{a}C(N—CN)NR^{a}R^{a}, —(CH₂)ₙ—NR^{a}C(NH)NR^{a}R^{a}, —(CH₂)ₙ—N═CR^{b}NR^{a}R^{a}, —(CH₂)ₙ—NR^{a}C(═O)NR^{a}R^{a}, —(CH₂)ₙ—C(═O)NR^{a}R^{a}, —(CH₂)ₙ—NR^{a}C(═S)NR^{a}C(═O)R^{b}, —(CH₂)ₙ—S(═O)ₚR^{c}, —(CH₂)ₙ—S(═O)ₚNR^{a}R^{a}, (CH₂)ₙ—NR^{a}S(═O)ₚNR^{a}R^{a}, —(CH₂)ₙ—NR^{a}S(═O)ₚR^{c}, —(CR^{d}R_{d})ₙ—C₃₋₁₀carbocyclyl substituted with 1-5 R⁵, and —(CR_{d}R_{d})ₙ-4- to 10-membered heterocyclyl substituted with 1-5 R⁵; optionally, two adjacent R³ groups on the carbocyclyl and heterocyclyl may form a ring substituted with 1-5 R⁵;

R^{3a} is independently selected from H and C₁₋₄alkyl;

R^{3b} is independently selected from H, C₁₋₄ alkyl substituted with 1-5 R⁵, C₂₋₄ alkenyl substituted with 1-5 R⁵, C₂₋₄ alkynyl substituted with 1-5 R⁵, —(CH₂)ₙ—CN, —(CH₂)ₙ—OR^{b}, —(CH₂)ₙ—OC(═O)NR^{a}R^{a}, —(CH₂))ₙ—NR^{a}R^{a}, —(CH₂)ₙ-C(═O)R^{b}), —(CH₂)ₙ—C(═O)OR^{b}, —(CH₂)ₙ—NR^{a}C(═O)OR^{b}, —(CH₂)ₙ—NR^{a}C(═O)R^{b}, —(CH₂)ₙ—NR^{a}C(N—CN)NR^{a}R^{a}, —(CH₂)ₙ—NR^{a}C(NH)NR^{a}R^{a}, —(CH₂)ₙ—N═CR^{b}NR^{a}R^{a}, —(CH₂)ₙ—NR^{a}C(═O)NR^{a}R^{a}, —(CH₂)ₙ—C(═O)NR^{a}R^{a}, —(CH₂)ₙ—NR^{a}C(═S)NR^{a}C(═O)R^{b}, —(CH₂)ₙ—S(═O)ₚR^{c}, —(CH₂)ₙ—S(═O),NR^{a}R^{a}, —(CH₂)ₙ—NR^{a}S(═O)ₚNR^{a}R^{a}, —(CH₂)ₙ—NR^{a}S(═O)ₚR^{c}, —(CR_{d}R_{d})ₙ—C₃₋₁₀ carbocyclyl substituted with 1-5 R⁵, and —(CR_{d}R^{d})ₙ-4- to 10-membered heterocyclyl substituted with 1-5 R⁵; optionally, two adjacent R³ groups on the carbocyclyl and heterocyclyl may form a ring substituted with 1-5 R⁵;

alternatively, R^{3a} and R^{3b} are taken together to form a C₃₋₆ carbocyclic or heterocyclic ring comprising carbon atoms and 1-3 heteroatoms selected from O, NR^{3c}, S, wherein the carbocyclic or heterocyclic ring is substituted with R^{3d};

alternatively, R^{3a} and R¹⁰ are taken together to form a C₃₋₆ carbocyclic or heterocyclic ring comprising carbon atoms and 1-3 hetero atoms selected from N, NR^{3e}, O, and S, wherein the carbocyclic or heterocyclic ring is substituted with R^{3d};

R^{3c} is independently selected from H, C₁₋₄alkyl substituted with 1-5 R⁵, —(CH₂)ₙ—C(═O)R^{b}, —(CH₂)ₙ—C(═O)OR^{b}, —(CH₂)ₙ—C(═O)NR^{a}R^{a}, —(CH₂)ₙ—NHC(═O)OR^{b}, —(CH₂)ₙ—S(═O)ₚR^{c}, —(CH₂)ₙ—S(═O)ₚNR^{a}R^{a}, —(CR_{d}R_{d})ₙ—C₃₋₁₀ carbocyclyl substituted with 1-5 R⁵, and —(CR_{d}R^{d})ₙ-4- to 10-membered heterocyclyl substituted with 1-5 R⁵;

R^{3d} is independently selected from H, NO₂, ═O, halogen, C₁₋₄alkyl substituted with 1-5 R⁵, C₂₋₄ alkenyl substituted with 1-5 R⁵. C₂₋₄ alkynyl substituted with 1-5 R⁵, CN, —(CH₂)ₙ—OR^{b}, —(CH₂)ₙ—NR^{a}R^{a}, —(CH₂)ₙ—C(═O)R^{b}, —(CH₂)ₙ—C(═O)OR^{b}, —(CH₂)ₙ—NR^{a}C(═O)OR^{b}, —(CH₂)ₐ—NR^{a}C(═O)R^{b}, —(CH₂)ₙ—NR^{a}C(N—CN)NR^{a}R^{a}, —(CH₂)ₙ—NR^{a}C(NH)NR^{a}R^{a}, —(CH₂)ₙ—N═CR^{b}NR^{a}R^{a}, —(CH₂)ₙ—NR^{a}C(═O)NR^{a}R^{a}, —(CH₂)ₙ—C(═O)NR^{a}R^{a}, —(CH₂)ₙ—NR^{a}C(═S)NR^{a}C(═O)R^{b}, —(CH₂)ₙ—S(═O)ₚR^{c}, —(CH₂)ₙ—S(═O)ₚNR^{a}R^{a}, —(CH₂)ₙ—NR^{a}S(═O)ₚNR^{a}R^{a}, —(CH₂)ₙ—NR^{a}S(═O)ₚR^{c}, —(CR_{d}R_{d})ₙ—C₃₋₁₀ carbocyclyl substituted with 1-5 R⁵, and —(CR_{d}R^{d})ₙ-4- to 10-membered heterocyclyl substituted with 1-5 R⁵:

We is independently selected from H and C₁₋₄alkyl;

alternatively, R^{3b} and R^{3e} are taken together to form a heterocyclic ring compring carbon atoms and 1-3 heteroatoms selected from O, NR^{3c}, S, wherein the heterocyclic ring is substituted with R^{3d};

R⁴ is independently selected from H, halogen, CN, C₁₋₆ alkyl substituted with 1-5 R⁹, and C₃₋₆ cycloalkyl substituted with 1-5 R⁹;

R⁵, at each occurrence, is independently selected from H, D, —(CH₂)ₙ—OR^{b}, ═O, —(CH₂)ₙ—NH₂, —(CH₂)ₙCN, halogen, C₁₋₆ alkyl, —(CH₂)ₙ—C(═O)OR^{b}, —(CH₂)ₙ—OR^{b}, —(CH₂)ₙ—C₃₋₁₀ carbocyclyl substituted with 0-5 R^{c}, —(CH₂)ₙ-4- to 10-membered heterocyclyl substituted with 0-5 R^{e}, and —O-4- to 10-membered heterocyclyl substituted with 0-5 R^{e};

R⁶ is independently selected from H, OH, ═O, —(CH₂)ₙNH₂, —(CH₂)ₙCN, halogen, C₁₋₆ alkyl, —(CH₂)ₙ—C(═O)OH, —(CH₂)ₙ—C(═O)OC₁₋₄alkyl, —(CH₂)ₙ—OC₁₋₄alkyl, —(CH₂)ₙ—C₃₋₁₀ carbocycle substituted with 0-5 R^{e}, —(CH₂)ₙ-4- to 10-membered heterocycle substituted with 0-5 R^{e}, and —(CH₂)ₙ-4- to 10-membered heterocycle substituted with 0-5 R^{c};

R⁷ is —(CH₂)ₙNH₂;

R⁸ is independently selected from H, OH, F, Cl, Br, C₁₋₄ alkyl, C₁₋₄alkoxy, CF₃, CN, C₃₋₆ cycloalkyl, aryl, and 5- to 6-membered heterocycle;

R⁹, at each occurrence, is independently selected from H, halogen and OH;

R¹⁰ is independently selected from H, C₁₋₄alkyl, and aryl;

R¹¹ is independently selected from H, halogen, CN, OCH₃, OCF₃, CH₃, C(═O)CH₃, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴;

R¹² is independently selected from H and halogen;

R¹³ is independently selected from H, F, Cl, methyl, ethyl, isopropyl, and OCH₃;

R¹⁴ is independently selected from H, F, and Cl;

R¹⁵ is independently selected from pyrazolyl, amninopyrazolyl, pyrrolidinyl, triazolonyl, aminothiadiazolyl, oxazolyl, thiazolyl, pyridinyl, pyrimidinyl, and 2-pyridonyl;

R¹⁶ is independently selected from H, CN, OR^{b}, halogen, NR^{a}R^{a}, and C₁₋₃ alkyl substituted with 0-5 R^{e};

R¹⁷ is independently selected from H, methyl and halogen;

R¹⁸ is independently selected from H, methyl and halogen;

R¹⁹ is independently selected from C₁₋₄alkyl, OC₁₋₄alkyl, SC₁₋₄ alkyl, and C₃₋₆ cycloalkyl;

R²⁰ is independently selected from H, halogen, CN, and C₁₋₄ alkyl;

R²¹ is independently selected from C₁₋₄alkyl, OC₁₋₄alkyl SC₁₋₄ alkyl, and C₃₋₆ cycloalkyl;

R²² is independently selected from C₁₋₄ alkyl and NR^{a}R^{a};

R²³ is independently selected from H, C₁₋₄ alkyl substituted with 0-3 R^{e}, CO₂H, —(CH₂)ₙCO₂(C₁₋₄alkyl), —(CH₂)ₙCO₂NH₂, —CONH(C₁₋₄ alkoxy), —CO₂(CH₂)₂O(C₁₋₄ alkyl), —CO₂(CH₂)₂N(C₁₋₄alkyl)₂, —CONH(CH₂)₂O(C₁₋₄alkyl), —CONH(CH₂)₂N(C₁₋₄alkyl)₂, —CON(C₁₋₄alkyl)(CH₂)₂O(C₁₋₄ alkyl), —CON(C₁₋₄alkyl)(CH₂)₂N(C₁₋₄alkyl); —CONHBn, —CONH(OBn), and —(CO)₀₋₁(CH₂)₀₋₃—C₃₋₆ carbocycle;

R²⁴, at each occurrence, is independently selected from H, halogen, CN, NO₂, ═O, C(O)NR^{a}R^{a}, C(═O)OR^{b}, Si(C₁₋₄alkyl)₃, —(CH₂)ₙ—OR^{b}, —(CH₂)ₙ—NR^{a}R^{a}, C(═NOH)NH₂, C₁₋₆ alkyl substituted with 0-5 R^{e}, C₂₋₆ alkenyl substituted with 0-5 R^{e}, C₂₋₆ alkynyl substituted with 0-5 R^{e}, aryl substituted with 0-5 R^{e}. —(CH₂)ₙ—C₃₋₆ cycloalkyl substituted with 0-5 R^{e}, —(CH₂)ₙ—O-4- to 10-membered heterocyclyl substituted with 0-5 R^{e};

R^{a}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};

R^{b}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH)ₙ-heterocyclyl substituted with 0-5 R^{e};

R^{c}, at each occurrence, is independently selected from C₁₋₆ alkyl substituted with 0-5 R^{e}, C₂₋₆alkenyl substituted with 0-5 R^{e}, C₂₋₆alkynyl substituted with 0-5 R^{e}, C₃₋₆carbocyclyl, and heterocyclyl;

R^{d}, at each occurrence, is independently selected from H and C₁₋₄alkyl substituted with 0-5 R^{e};

R^{e}, at each occurrence, is independently selected from F, Cl, Br, CN, NO₂, —O; C₁₋₆ alkyl substituted with 0-5 R^{f}, C₂₋₆ alkenyl, C₂₋₆ alkynyl, —(CH₂)ₙ—C₃₋₆ cycloalkyl, —(CH₂)ₙ-aryl, —(CH₂)ₙ-heterocyclyl, CO₂H, —(CH₂)ₙOR^{f}, SR^{f}, and —(CH₂)ₙNR^{f}R^{f};

R^{f}, at each occurrence, is independently selected from H, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl;

n, at each occurrence, is an integer independently selected from 0, 1, 2, and 4; and

p, at each occurrence, is an integer independently selected from 0, 1, and 2.

In a second aspect, the present disclosure provides a compound of Formula (I), or a stereoisomer, a tautomer, a pharmaceutically acceptable salt, or a solvate thereof, within the scope of the first aspect, wherein:

Ma is (M1-I):

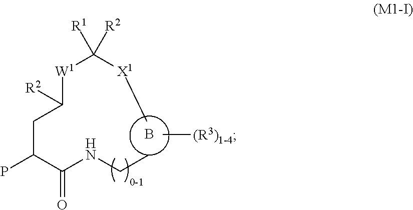

P is independently selected from Formulae (P1), (P2), (P3), (P4), (P5), (P6), (P7), (P8), P(9), P(10), P(11), P(12), P(13), and P(14):

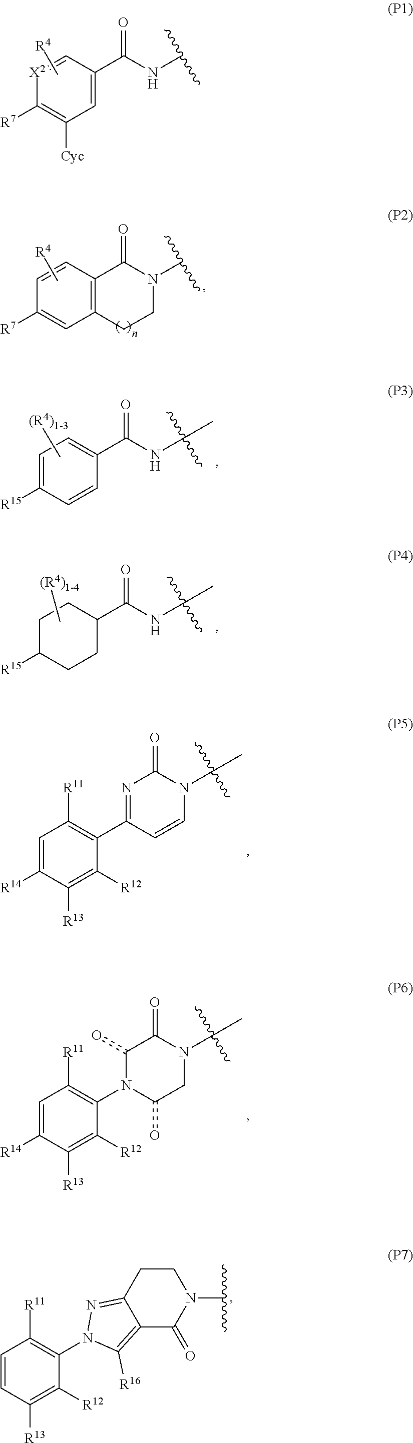

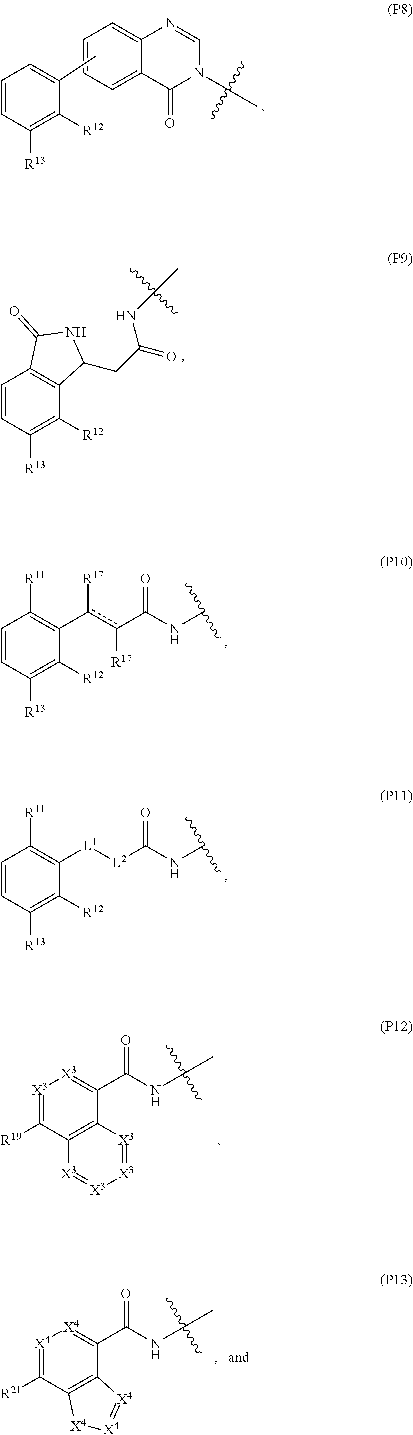

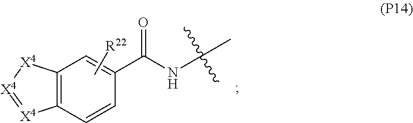

Cyc is C₃₋₆ cycloalkyl;

Ring B is independently selected from aryl and 5- to 6-membered heterocyclyl;

W¹ is independently selected from (CHR^{1a})₁₋₂, O, NH, and N(C₁₋₄ alkyl);

X¹ is independently selected from NHC(═O)— and C(═O)NH—; provided when W¹ is O, NH, and N(C₁₋₄alkyl), X¹ is not —NHC(═O)—;

X² is independently selected from N and CR⁴;

X³ is independently selected from CR¹⁸ and N;

X⁴ is independently selected from CR²⁰, O, S, N, NH, and. NC₁₋₄alkyl;

L¹ is independently selected from CH₂, O, NH, NC₁₋₄alkyl, and S;

L² is independently selected from NH and NC₁₋₄alkyl;

R¹ is independently selected from H and C₁₋₄alkyl;

R^{1a}is independently selected from H, F, CH₃, and OH;

R² is independently selected from H and OH;

R³ is independently selected from H, ═O, halogen, C₁₋₄alkyl substituted with 1-5 R⁵, CN, —OR^{b}, —(CH₂)ₙ—C(═O)R^{b}, —(CH₂)ₙ—C(═O)OR^{b}, —NR^{a}C(═O)OR^{b}, —NR^{a}C(═O)R^{b}, —NR^{a}C(═O)NR^{a}R^{a}, —C(═O)NR^{a}R^{a}, —S(═O)ₚR^{c}, —(CH₂)ₙ—C₃₋₁₀ carbocyclyl substituted with 1-5 and —(CH₂)ₙ-4- to 10-membered heterocyclyl substituted with 1-5 R⁵; optionally, two adjacent R³ groups on the carbocyclyl and heterocyclyl may form a ring substituted with 1-5 R⁵;

R⁴ is independently selected from H, halogen, CN, C₁₋₆ alkyl substituted with 1-5 R⁹, and C₃₋₆ cycloalkyl substituted with 1-5 R⁹;

R⁵, at each occurrence, is independently selected from H, D, —(CH₂)ₙ—OR^{b}, ═O, —(CH₂)ₙNH₂, —(CH₂)ₙCN, halogen, C₁₋₆ alkyl, —(CH₂)ₙ—C(═O)OR^{b}, —(CH₂)ₙ—OR^{b}, —(CH₂)ₙ—C₃₋₁₀ carbocyclyl substituted with 0-5 R^{e}, —(CH₂)ₙ-4- to 10-membered heterocyclyl substituted with 0-5 R^{e}, and —O-4- to 10-membered heterocyclyl substituted with 0-5 R^{e},

R⁷ is —(CH₂)ₙNH₂;

R⁹, at each occurrence, is independently selected from H, halogen and OH;

R¹¹ is independently selected from H, halogen, CN, OCH₃, OCF₃, CH₃, C(═O)CH₃, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴;

R¹² is independently selected from H and halogen;

R¹³ is independently selected from H, F, Cl, methyl, ethyl, isopropyl, and OCH₃;

R¹⁴ is independently selected from H, F, and Cl;

R¹⁵ is independently selected from pyrazolyl, aminopyrazolyl, pyrrolidinyl, triazolonyl, aminothiadiazolyl oxazolyl, thiazolyl, pyridinyl, pyrimidinyl, and 2-pyridonyl;

R¹⁶ is independently selected from H, CN, OR^{b}, halogen, NR^{a}R^{a}, and C₁₋₃ alkyl substituted with 0-5 R^{e};

R¹⁷ is independently selected from H, methyl and halogen;

R¹⁸ is independently selected from H, methyl and halogen;

R¹⁹ is independently selected from C₁₋₄alkyl, OC₁₋₄ alkyl, SC₁₋₄ alkyl, and C₃₋₆ cycloalkyl;

R²⁰ is independently selected from H, halogen, CN, and C₁₋₄alkyl;

R²¹ is independently selected from C₁₋₄ alkyl, OC₁₋₄alkyl, SC₁₋₄ alkyl, and C₃₋₆ cycloalkyl;

R²² is independently selected from C₁₋₄alkyl and NR^{a}R^{a};

R²⁴, at each occurrence, is independently selected from H, halogen, CN, NO₂, ═O, C(═O)NR^{a}R^{a}, C(═O)OR^{b}, —(CH₂)ₙ—OR^{b}, —(CH₂)ₙ—NR^{a}R^{a}, C(═NOH)NH₂, C₁₋₆ alkyl substituted with 0-5 R^{e}, C₂₋₆ alkenyl substituted with 0-5 R^{e}, C₂₋₆ alkynyl substituted with 0-5 R^{e}, aryl substituted with 0-5 R^{e}, —(CH₂)ₙ—C₃₋₆cycloalkyl substituted with 0-5 R^{e}, —(CH₂)ₙ—O-4- to 10-membered heterocyclyl substituted with 0-5 R^{e};

R^{e}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};

R^{b}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e};

R^{c}, at each occurrence, is independently selected from C₁₋₆ alkyl substituted with 0-5 R^{e}, C₂₋₆alkenyl substituted with 0-5 R^{e}, C₂₋₆alkynyl substituted with 0-5 R^{e}, C₃₋₆carbocyclyl, and heterocyclyl;

R^{d}, at each occurrence, is independently selected from H and C₁₋₄alkyl substituted with 0-5 R^{e};

R^{e}, at each occurrence, is independently selected from F, Cl, Br, CN, NO₂, ═O, C₁₋₆alkyl substituted with 0-5 R^{f}, C₂₋₆ alkenyl, C₂₋₆ alkynyl, —(CH₂)ₙ—C₃₋₆ cycloalkyl, —(CH₂)ₙ-aryl, —(CH₂)ₙ-heterocyclyl, CO₂H, —(CH₂)ₙOR^{f}, SR^{f}, and —(CH₂)ₙNR^{f}R^{f};

R^{f}, at each occurrence, is independently selected from F₁, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl;

n, at each occurrence, is an integer independently selected from 0, 1, 2, and 4; and

p, at each occurrence, is an integer independently selected from 0, 1, and 2.

In a third aspect, the present disclosure provides a compound of Formula (I) or a stereoisomer, a tautomer, a pharmaceutically acceptable salt, or a solvate thereof, within the scope of the first or second aspect, wherein:

ring B is independently selected from

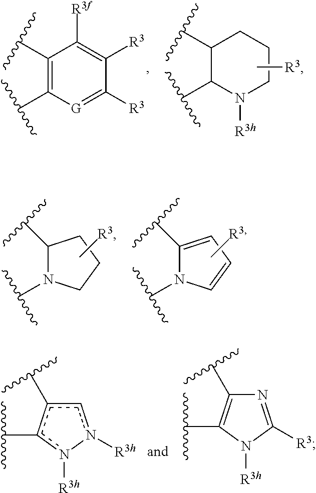

G is independently selected from N and CR^{3g};

R³ is independently selected from H, ═O, halogen, C₁₋₄alkyl substituted with 1-5 R⁵, CN, —NR^{a}R^{a}, —C(═O)R^{b}, —C(═O)OR^{b}, —NR^{a}C(═O)OR^{b}, —NR^{a}C(═O)R^{b}, —NR^{a}C(═O)NR^{a}R^{a}, —C(═O)NR^{a}R^{a}, —S(═O)ₚR^{c}, —(CH₂)ₙ—C₃₋₁₀ carbocyclyl substituted with 1-5 R⁵, and —(CH₂)ₙ-4- to 10-membered heterocyclyl substituted with 1-5 R⁵;

R^{3f} is independently selected from H and halogen;

R^{3g} is independently selected from H, halogen, methyl, and CN;

R^{3h} is absent or independently selected from H, C₁₋₄alkyl substituted with 1-4 R⁵, —(CH₂)ₙ—C(═O)R^{b}, —(CH₂)ₙ—C(═O)OR^{b}; and

R⁵, at each occurrence, is independently selected from H, D, —OR^{b}, —NH₂, —CN, halogen, C₁₋₆ alkyl, —C(═O)OR^{b}, C₃₋₁₀ carbocyclyl substituted with 0-5 R^{e}, -4- to 10-membered heterocyclyl substituted with 0-5 R^{e}, and —O-4- to 10-membered heterocyclyl substituted with 0-5 R^{e}.

In a fourth aspect, the present disclosure provides a compound of Formula (I) or a stereoisomer, a tautomer, a pharmaceutically acceptable salt, or a solvate thereof, within the scope of any of the first, second and third aspects, wherein:

Ma is (M2-I):

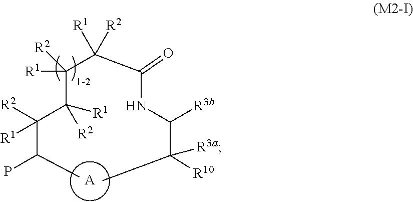

P is independently selected from Formulae (P1), (P2), (P3), (P4), (P5), (P6), (P7), (P8), P(9), P(10), P(11), P(12), P(13), and P(14):

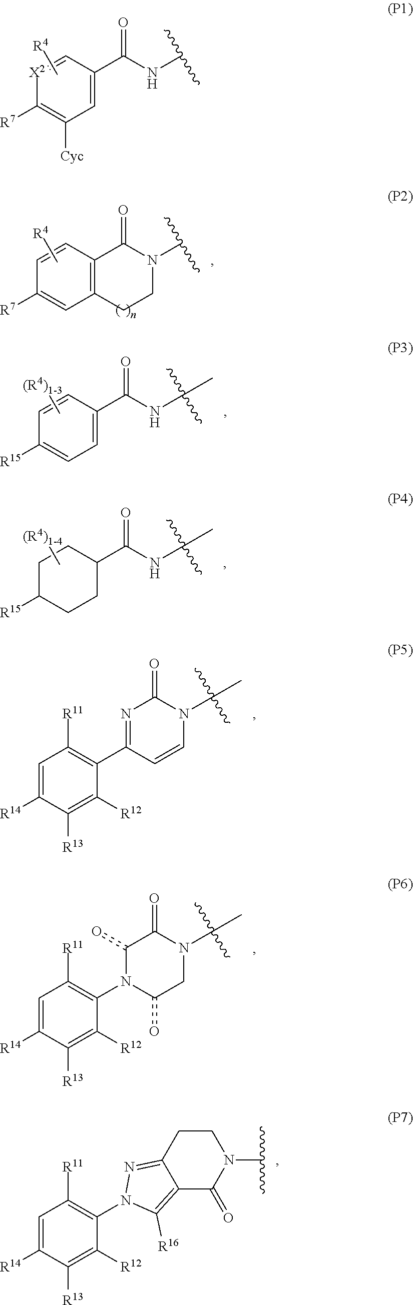

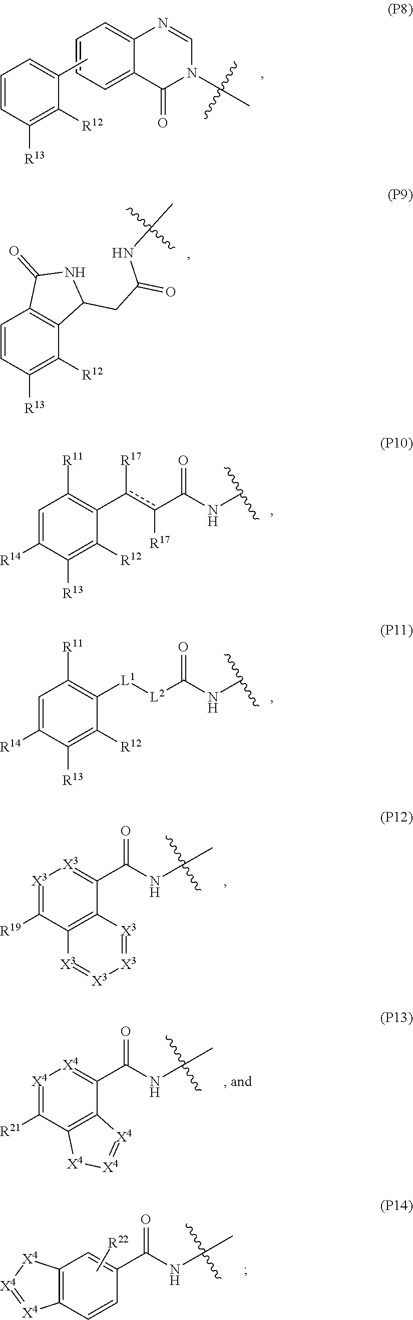

Cyc is C₃₋₆ cycloalkyl;

ring A is independently selected from

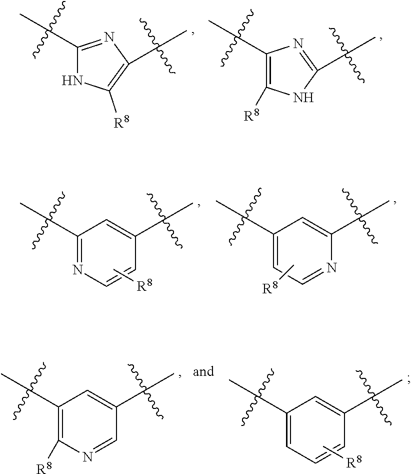

X² is independently selected from N and CR⁴;

X³ is independently selected from CR¹⁸ and N;

X⁴ is independently selected from CR²⁰, O, S, N, NH, and NC₁₋₄alkyl;

L¹ is independently selected from CH₂, O, NH, NC₁₋₄alkyl, and S;

L² is independently selected from NH and NC₁₋₄alkyl;

R¹ and R² are independently selected from H, halogen, C₁₋₄alkyl, and OH;

alternatively, two adjacent R′ groups form a bond;

R^{3a} is independently selected from H and C₁₋₄alkyl;

R^{3b} is independently selected from —(CH₂)ₙ—CN, —(CH₂)ₙ—OR^{b}, —(CH₂)ₙ—OC(═O)NR^{a}R^{a}, —(CH₂)ₙ—NR^{a}R^{a}, —(CH₂)ₙ—C(═O)R^{b}, —(CH₂)ₙ—C(═O)OR^{b}, —(CH₂)ₙ—NR^{a}C(═O)OR^{b}, —(CH₂)ₙ—NR^{a}C(═O)R^{b}, —NR^{a}C(═O)NR^{a}R^{a}, —C(═O)NR^{a}R^{a};

alternatively, R^{3b} and R^{3b} are taken together to form a heterocyclic ring selected from

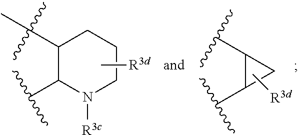

alternatively, R^{3a} and R¹⁰ are taken together to form a heterocyclic ring comprising carbon atoms and 1-3 NR^{3c}, wherein the heterocyclic ring is substituted with R^{3d};

R^{3c} is independently selected from H, C₁₋₄ alkyl substituted with 1-5 R⁵, —(CH₂)ₙ—C(═O)R^{b}, —(CH₂)ₙ—C(═O)OR^{b}, —(CH₂)ₙ—C(═O)NR^{a}R^{a}, —(CH₂)ₙ—NHC(═O)OR^{b}, —(CH₂)ₙ—S(═O)ₚR^{c}, —(CH₂)ₙ—S(═O)ₚNR^{a}R^{a}, —(CR_{d}R_{d})ₙ—C₃₋₁₀ carbocyclyl substituted with 1-5 R⁵, and —(CR_{d}R_{d})ₐ-4- to 10-membered heterocyclyl substituted with 1-5 R⁵;

R^{3d} is independently selected from H, ═O, and C₁₋₄ alkyl substituted with 1-5 R⁵;

R⁴ is independently selected from H, halogen, CN, C₁₋₆ alkyl substituted with 1-5 R⁹, and C₃₋₆ cycloalkyl substituted with 1-5 R⁹;

R⁵, at each occurrence, is independently selected from H, D, —(CH₂)ₙ—OR^{b}, ═O, —(CH₂)ₙNH₂, —(CH₂)ₙCN, halogen, C₁₋₆ alkyl, —(CH₂)ₙ—C(═O)OR^{b}, —(CH₂)ₙ—OR^{b}, —(CH₂)ₙ—C₃₋₁₀ carbocyclyl substituted with 0-5 R^{e}, —(CH₂)ₙ-4- to 10-membered heterocyclyl substituted with 0-5 R^{e}, and —O-4- to 10-membered heterocyclyl substituted with 0-5 R^{e};

R⁷ is —(CH₂)ₙNH₂;

R⁸ is independently selected from H, OH, F, Cl, Br, C₁₋₄alkyl, C₁₋₄alkoxy, CF₃, CN, C₃₋₆ cycloalkyl, aryl, and 5- to 6-membered heterocycle;

R⁹, at each occurrence, is independently selected from H, halogen and OH;

R¹⁰ is independently selected from H, C₁₋₄alkyl, and aryl;

R¹¹ is independently selected from H, halogen, CN, OCH₃, OCF₃, CH₃, C(═O)CH₃, CHF₂, CF₃, CCH₃F₂, OCHF₂, aryl, C₃₋₆cycloalkyl, and 4-6 membered heterocycle, wherein said aryl, cycloalkyl and heterocycle is optionally substituted with R²⁴;

R¹² is independently selected from H and halogen;

R¹³ is independently selected from H, F, Cl, methyl, ethyl, isopropyl, and OCH₃;

R¹⁴ is independently selected from H, F, and Cl;

R¹⁵ is independently selected from pyrazolyl, aminopyrazolyl, pyrrolidinyl, triazolonyl, aminothiadiazolyl, oxazolyl, thiazolyl, pyridinyl, pyrimidinyl, and 2-pyridonyl;

R¹⁶ is independently selected from H, CN, OR^{b}, halogen, NR^{a}R^{a}, and C₁₋₃ alkyl substituted with 0-5 R^{e};

R¹⁷ is independently selected from H, methyl and halogen;

R¹⁸ is independently selected from H, methyl and halogen;

R¹⁹ is independently selected from C₁₋₄alkyl, OC₁₋₄alkyl, SC₁₋₄ alkyl, and C₃₋₆ cycloalkyl;

R²⁰ is independently selected from H, halogen, CN, and C₁₋₄alkyl;

R²¹ is independently selected from C₁₋₄alkyl, OC₁₋₄alkyl, SC₁₋₄alkyl, and C₃₋₆ cycloalkyl;

R²² is independently selected from C₁₋₄ alkyl and NR^{a}R^{a};

R²⁴, at each occurrence, is independently selected from H, F, Cl, Br, C(═O)NR^{a}R^{a}, C(═O)OR^{b}, —(CH₂)ₙ—OR^{b}, —(CH₂)ₙ—NR^{a}R^{a}, C₁₋₆ alkyl substituted with 0-5 R^{e}, aryl substituted with 0-5 R^{e}, —(CH₂)ₙ—C₃₋₆cycloalkyl substituted with 0-5 R^{e}, —(CH₂)ₙ—O-4- to 10-membered heterocyclyl substituted with 0-5 R^{e};

R^{a}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};

R^{b}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e};

R^{c}, at each occurrence, is independently selected from C₁₋₆ alkyl substituted with 0-5 R^{e}, C₂₋₆alkenyl substituted with 0-5 R^{e}, C₂₋₆alkynyl substituted with 0-5 R^{e}, C₃₋₆carbocyclyl, and heterocyclyl;

R^{d}, at each occurrence, is independently selected from H and C₁₋₄alkyl substituted with 0-5 R^{e};

R^{e}, at each occurrence, is independently selected from F, Cl, Br, CN, NO₂, ═O, C₁₋₆ alkyl substituted with 0-5 R^{f}, C₂₋₆ alkenyl, C₂₋₆ alkynyl, —(CH₂)ₙ—C₃₋₆ cycloalkyl, —(CH₂)ₙ-aryl, —(CH₂)ₙ-heterocyclyl, CO₂H, —(CH₂)ₙOR^{f}, SR^{f}, and —(CH₂)ₙNR^{f}R^{f};

R^{f}, at each occurrence, is independently selected from H, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl;

n, at each occurrence, is an integer independently selected from 0, 1, 2, and 4; and

p, at each occurrence, is an integer independently selected from 0, 1, and 2.

In a fifth aspect, the present disclosure provides a compound of Formula (I), or a stereoisomer, a tautomer, a pharmaceutically acceptable salt, or a solvate thereof, within the scope of any of the first, second, third, and fourth aspects, wherein:

Ma is (M2-II):

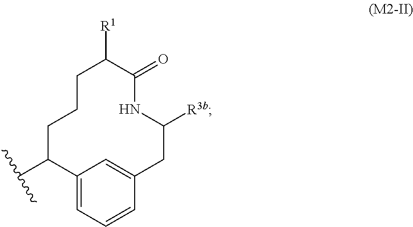

P is independently selected from Formulae (P1), (P2), (P3), (P4), (P5), (P6), (P7), (P8), P(9), P(10), P(11), P(12), P(13) and P(14):

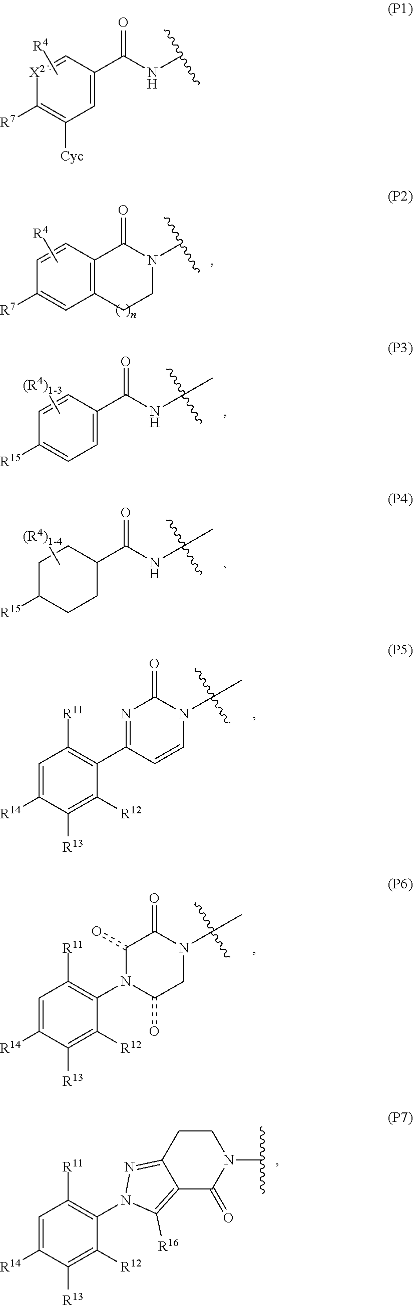

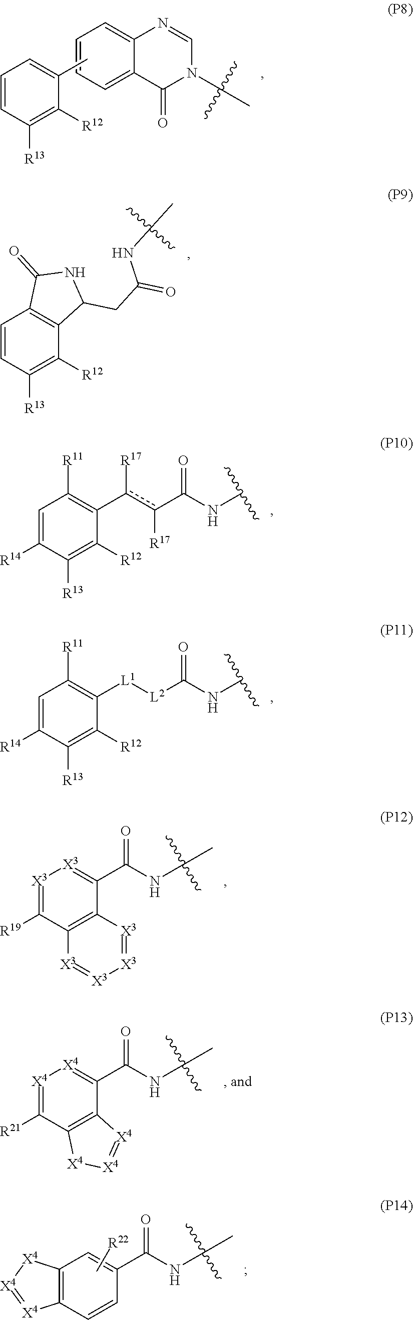

Cyc is C₃₋₆ cycloalkyl;

X² is independently selected from N and CR⁴;

X³ is independently selected from CR¹⁸and N;

X⁴ is independently selected from CR²⁰, O, S, N, NH, and NC₁₋₄alkyl;

L¹ is independently selected from CH₂, O, NH, NC₁₋₄alkyl, and S;

L² is independently selected from NH and NC₁₋₄alkyl;

R¹ is independently selected from H and C₁₋₄alkyl;

R^{3b} is independently selected from —(CH₂)ₙ—CN, —(CH₂)ₙ—OR^{b}, —(CH₂)ₙ—OC(═O)NR^{a}R^{a}, —C(═O)OR^{b}, —(CH₂)ₙ—NR^{a}C(═O)OR^{b}, —(CH₂)ₙ—NR^{a}C(═O)R^{b}, —NR^{a}C(═O)NR^{a}R^{a}, and —C(═O)NR^{a}R^{a};

R⁴ is independently selected from H, halogen, CN, C₁₋₆ alkyl substituted with 1-5 R⁹, and C₃₋₆ cycloalkyl substituted with 1-5 R⁹;

R⁵, at each occurrence, is independently selected from H, D, —(CH₂)ₙ—OR^{b}, ═O, —(CH₂)ₙNH₂, —(CH₂)ₙCN, halogen, C₁₋₆ alkyl, —(CH₂)ₙ—C(═O)OR^{b}, —(CH₂)ₙ—OR^{b}, —(CH₂)ₙ—C₃₋₁₀ carbocyclyl substituted with 0-5 R^{e}, —(CH₂)ₙ-4- to 10-membered heterocyclyl substituted with 0-5 R^{e}, and —O-4- to 10-membered heterocyclyl substituted with 0-5 R^{e};

R⁷ is —(CH₂)ₙNH₂,

R⁹, at each occurrence, is independently selected from H, halogen and OH;

R¹⁰ is independently selected from H, C₁₋₄alkyl, and aryl;

R¹¹ is independently selected from H, halogen, CN, OCH₃, OCF₃, CH₃, C(═O)CH₃, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴;

R¹² is independently selected from H and halogen;

R¹³ is independently selected from H, F, Cl, methyl, ethyl, isopropyl, and OCH₃;

R¹⁴ is independently selected from H, F, and Cl;

R¹⁵ is independently selected from pyrazolyl, aminopyrazolyl, pyrrolidinyl, triazolonyl, aminothiadiazolyl, oxazolyl, thiazolyl, pyridinyl, pyrimidinyl, and 2-pyridonyl;

R¹⁶ is independently selected from H, CN, Or^{b}, halogen, NR^{a}R^{a}, and C₁₋₃ alkyl substituted with 0-5 R^{e};

R¹⁷ is independently selected from H, methyl and halogen;

R¹⁸ is independently selected from H, methyl and halogen;

R¹⁹ is independently selected from C₁₋₄alkyl, OC₁₋₄ alkyl, SC₁₋₄ alkyl, and C₃₋₆ cycloalkyl;

R²⁰ is independently selected from H, halogen, CN, and C₁₋₄ alkyl;

R²¹ is independently selected from C₁₋₄alkyl, OC₁₋₄ alkyl, SC₁₋₄alkyl, and C₃₋₆ cycloalkyl;

R²² is independently selected from C₁₋₄alkyl and NR^{a}R^{a};

R²⁴, at each occurrence, is independently selected from H, F, Cl, Br, C(═O)NR^{a}R^{a}, C(═O)OR^{b}, —(CH₂)ₙ—OR^{b}, —(CH₂)ₙ—NR^{a}R^{a}, C₁₋₆ alkyl substituted with 0-5 R^{e}, aryl substituted with 0-5 R^{e}, —(CH₂)ₙ—C₃₋₆ cycloalkyl substituted with 0-5 R^{e}, —(CH₂)ₙ—O-4- to 10-membered heterocyclyl substituted with 0-5 R^{e};

R^{a}, at each occurrence, is independently selected from C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₂₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};

R^{b}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R_{c}, —(CH₂)ₙ—C₃₋₁₀carbocyclyl substituted with 0-5 R^{e}, and —(CH₂)ₙ-heterocyclyl substituted with 0-5 R^{e};

R^{e}, at each occurrence, is independently selected from F, Cl, Br, CN, NO₂, ═O, C₁₋₆ alkyl substituted with 0-5 R^{f}, C₂₋₆ alkenyl, C₂₋₆ alkynyl, —(CH₂)ₙ-C₃₋₆ cycloalkyl, —(CH₂)ₙ-aryl, —(CH₂)ₙ-heterocyclyl, CO₂H, —(CH₂)ₙOR^{f}, SR^{f}. and —(CH₂)ₙNR^{f}R^{f};

R^{f}, at each occurrence, is independently selected from H, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl; and

n, at each occurrence, is an integer independently selected from 0, 1, 2, and 4,

In a sixth aspect, the present disclosure provides a compound of Formula (I) or a stereoisomer, a tautomer, a pharmaceutically acceptable salt, or a solvate thereof, within the scope of any of the first, second, third and fourth aspects, wherein:

Ma is (M2-II):

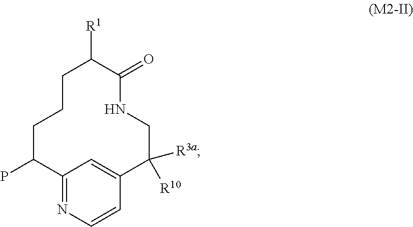

P is independently selected from Formulae (P1), (P2), (P3), (P4), (P5), (P6), (P7), (P8), P(9), P(10), P(11), P(12), P(13), and P(14):

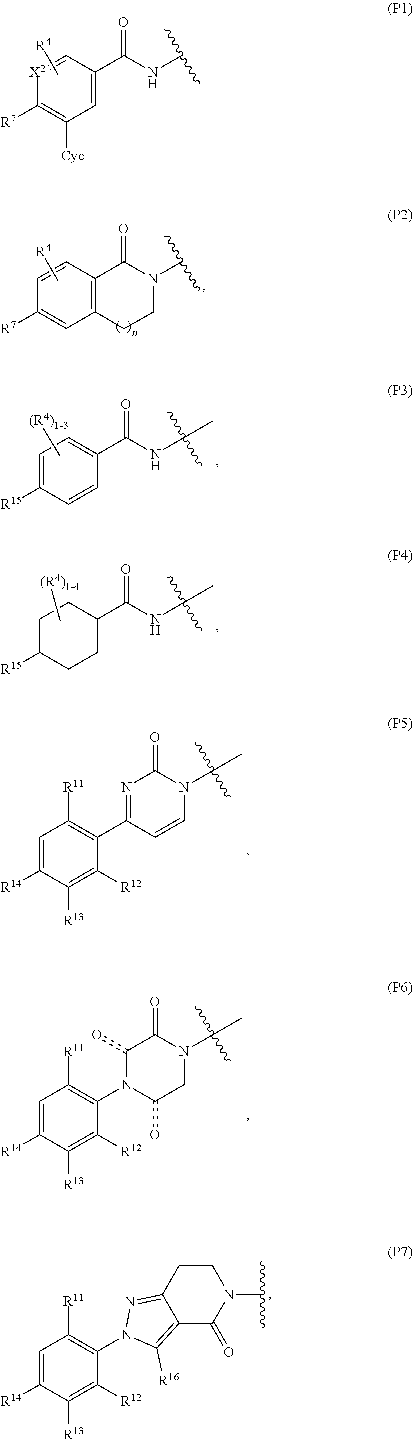

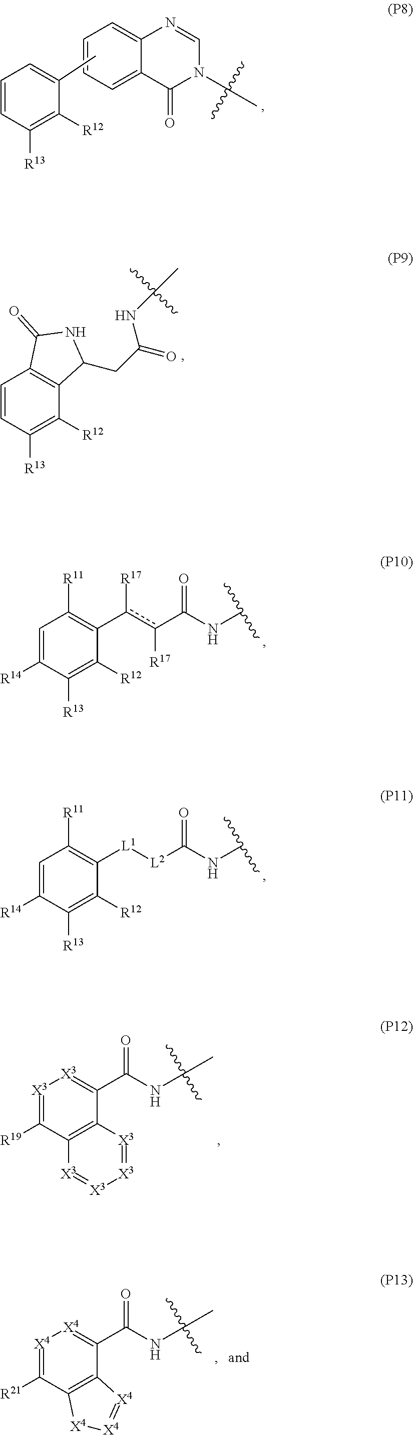

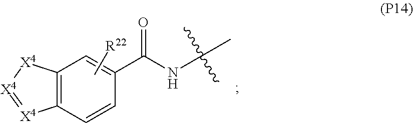

Cyc is C₃₋₆ cycloalkyl;

X² is independently selected from N and CR⁴;

X³ is independently selected from CR¹⁸ and N;

X⁴ is independently selected from CR²⁰, O, S, N, NH, and NC₁₋₄alkyl;

L¹ is independently selected from CH₂, O, NH, NC₁₋₄alkyl, and S;

L² is independently selected from NH and NC₁₋₄alkyl;

R¹ is independently selected from H and C₁₋₄alkyl;

R^{3a} is independently selected from H and C₁₋₄ alkyl;

alternatively, R^{3a} and R¹⁰ are taken together to form

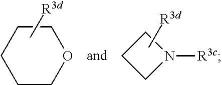

R^{3c} is independently selected from H, C₁₋₄alkyl substituted with 1-5 R⁵, —(CH₂)ₙ—C(═O)R^{b}, —(CH₂)ₙ—C(═O)OR^{b}, —(CH₂)ₙ—C(═O)NR^{a}R^{a}, and —(CH₂)ₙ—NHC(═O)OR^{b},

R^{3d} is independently selected from H and ═O;

R⁴ is independently selected from H, halogen, CN, C₁₋₆alkyl substituted with 1-5 R⁹, and C₃₋₆ cycloalkyl substituted with 1-5 R⁹;

R⁵, at each occurrence, is independently selected from H, D, —(CH₂)ₙ-OR^{b}, ═O, —(CH₂)ₙNH₂, —(CH₂)ₙCN, halogen, C₁₋₆ alkyl, —(CH₂)ₙ—C(═O)OR^{b}, —(CH₂)ₙ—OR^{b}, —(CH₂)ₙ—C₃₋₁₀ carbocyclyl substituted with 0-5 R^{e}, —(CH₂)ₙ-4- to 10-membered heterocyclyl substituted with 0-5 R^{e}, and —O-4- to 1.0-membered heterocyclyl substituted with 0-5 R^{e};

R⁶ is independently selected from H, OH, ═O, —(CH₂)ₙNH₂, —(CH₂)ₙCN, halogen, C₁₋₆ alkyl, —(CH₂)ₙ—C(═O)OH, —(CH₂)ₙ—C(═O)OC₁₋₄alkyl, —(CH₂)ₙ—OC₁₋₄ alkyl, —(CH₂)ₙ—C₃₋₁₀carbocycle substituted with 0-5 R^{e}, —(CH₂)ₙ-4- to 10-membered heterocycle substituted with 0-5 R^{e}, and —(CH₂)ₙ-4- to 10-membered heterocycle substituted with 0-5 R^{e};

R⁷ is —(CH₂)ₙNH₂;

R⁹, at each occurrence, is independently selected from H, halogen and OH;

R¹⁰ is independently selected from H, C₁₋₄alkyl, and aryl;

R¹¹ is independently selected from H, halogen, CN, OCH₃, OCF₃, CH₃, C(═O)CH₃, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴;

R¹² is independently selected from H and halogen;

R¹³ is independently selected from H, F, Cl, methyl, ethyl, isopropyl, and OCH₃;

R¹⁴ is independently selected from H, F, and Cl;

R¹⁵ is independently selected from pyrazolyl, aminopyrazolyl, pyrrolidinyl, triazolonyl, aminothiadiazolyl, oxazolyl, thiazolyl, pyridinyl, pyrimidinyl, and 2-pyridonyl;

R¹⁶ is independently selected from H, CN, OR^{b}, halogen, NR^{a}R^{a}, and C₁₋₃ alkyl substituted with 0-5 R^{e};

R¹⁷ is independently selected from H, methyl and halogen;

R¹⁸ is independently selected from H, methyl and halogen;

R¹⁹ is independently selected from C₁₋₄ alkyl, OC₁₋₄ alkyl, SC₁₋₄alkyl, and C₃₋₆ cycloalkyl;

R²⁰ is independently selected from H, halogen, CN, and C₁₋₄alkyl;

R²¹ is independently selected from C₁₋₄alkyl, OC₁₋₄ alkyl, SC₁₋₄ alkyl, and C₃₋₆ cycloalkyl;

R²² is independently selected from C₁₋₄ alkyl and NR^{a}R^{a};

R²⁴, at each occurrence, is independently selected from H, F, Cl, Br, C(═O)NR^{a}R^{a}, C(O)OR^{b}, —(CH₂)ₙ—OR^{b}, —(CH₂)ₙ—NR^{a}R^{a}, C₁₋₆ alkyl substituted with 0-5 R^{e}, aryl substituted with 0-5 R^{e}, —(CH₂)ₙ—C₃₋₆ cycloalkyl substituted with 0-5 R^{e}, —(CH₂)ₙ—O-4- to 10-membered heterocyclyl substituted with 0-5 R^{e};

R^{a}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};

R^{b}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e};

R^{c}, at each occurrence, is independently selected from F, Cl, Br, CN, NO₂, ═O, C₁₋₆ alkyl substituted with 0-5 C₂₋₆ alkenyl, C₂₋₆ alkynyl, cycloalkyl, —(CH₂)ₙ-aryl, —(CH₂)ₙ-heterocyclyl, CO₂H, —(CH₂)ₙOR^{f}, SR^{f}, and —(CH₂)ₙNR^{f}R^{f};

R^{f}, at each occurrence, is independently selected from H, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl; and

n, at each occurrence, is an integer independently selected from 0, 1, 2, and 4,

In a seventh aspect, the present disclosure provides a compound of Formula (I) or a stereoisomer, a tautomer, a pharmaceutically acceptable salt, or a solvate thereof, within the scope of any of the first, second, third, and fourth aspects, wherein:

Ma is (M2-III):

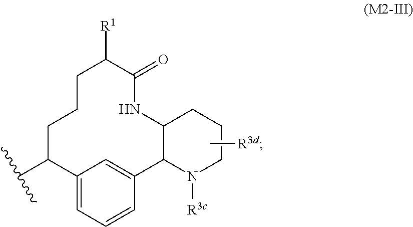

P is independently selected from Formulae (P1), (P2), (P3), (P4), (P5), (P6), (P7), (P8), P(9), P(10), P(11), P(12), P(13), and P(14):

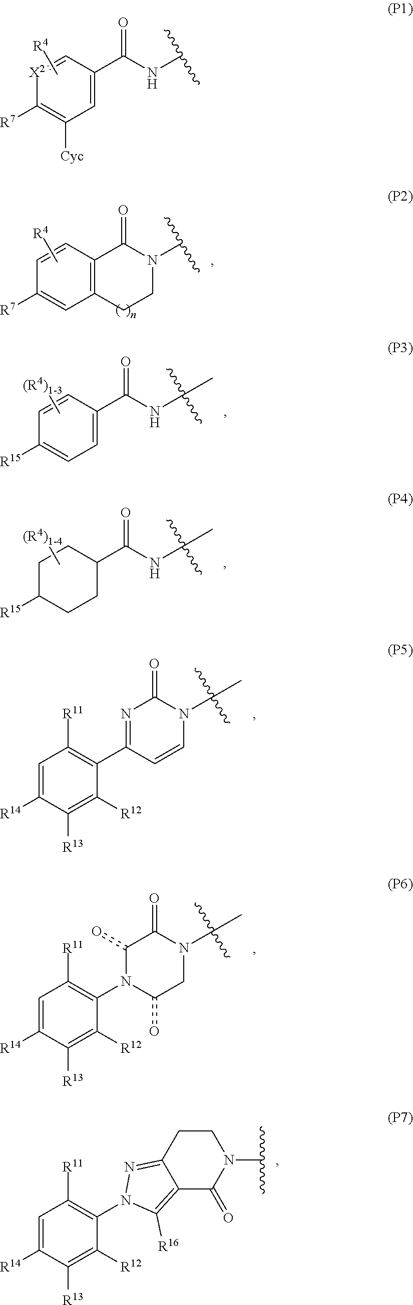

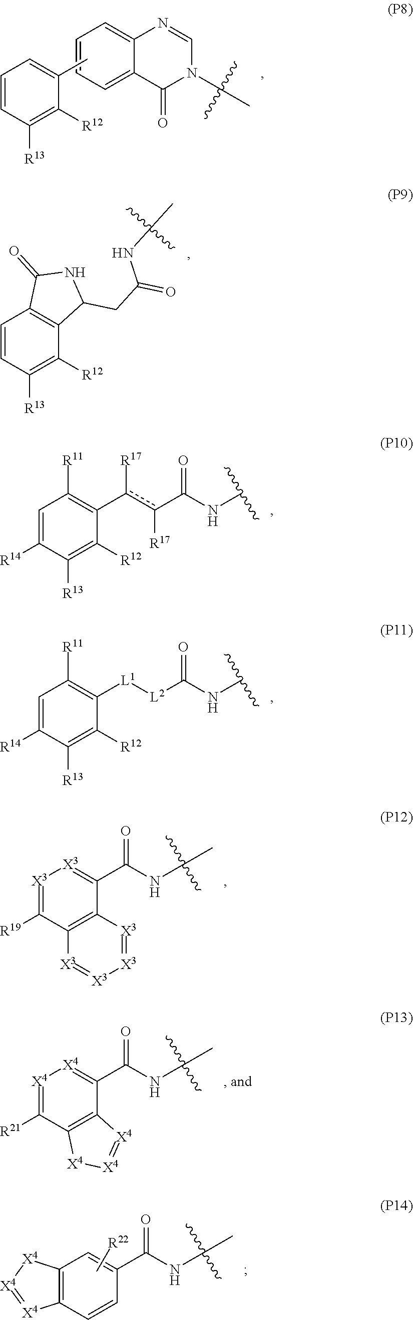

Cyc is C₃₋₆ cycloalkyl;

X² is independently selected from N and CR⁴;

X³ is independently selected from CR¹⁸ and N;

X⁴ is independently selected from CR²⁰, O, S, N, NH, and NC₁₋₄alkyl;

L¹ is independently selected from CH₂, O, NH, NC₁₋₄alkyl, and S;

L² is independently selected from NH and NC₁₋₄alkyl;

R¹ is independently selected from H and C₁₋₄alkyl;

R^{3c} is independently selected from H, C₁₋₄alkyl, —C(═O)C₁₋₄alkyl, —(CH₂)ₙ—C(═O)OH, and —(CH₂)ₙ—C(═O)OC₁₋₄alkyl;

R^{3d} is independently selected from H and ═O;

R⁴ is independently selected from H, halogen, CN, C₁₋₆ alkyl substituted with 1-5 R⁹, and C₃₋₆ cycloalkyl substituted with 1-5 R⁹;

R⁵, at each occurrence, is independently selected from H, D, —(CH₂)ₙ—OR^{b}, ═O, —(CH₂)ₙNH₂, —(CH₂)ₙCN, halogen, C₁₋₆ alkyl, —(CH₂)ₙ—C(═O)OR^{b}, —(CH₂)ₙ—OR^{b}, —(CH₂)ₙ—C₃₋₁₀ carbocyclyl substituted with 0-5 R^{e}, —(CH₂)ₙ-4- to 10-membered heterocyclyl substituted with 0-5 R^{e}, and —O-4- to 10-membered heterocyclyl substituted with 0-5 R^{e};

R⁷ is —(CH₂)ₙNH₂;

R⁹, at each occurrence, is independently selected from H, halogen and OH;

R₁₁ is independently selected from H, halogen, CN, OCH₃, OCF₃, CH₃, C(═O)CH₃, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴;

R¹² is independently selected from H and halogen;

R¹³ is independently selected from H, F, Cl, methyl, ethyl, isopropyl, and OCH₃;

R¹⁴ is independently selected from H, F, and Cl;

R¹⁵ is independently selected from pyrazolyl, amninopyrazolyl, pyrrolidinyl, triazolonyl, aminothiadiazolyl, oxazolyl, thiazolyl, pyridinyl, pyrimidinyl, and 2-pyridonyl;

R¹⁶ is independently selected from H, CN, OR^{b}, halogen, NR^{a}R^{a}, and C₁₋₃ alkyl substituted with 0-5 R^{e};

R¹⁷ is independently selected from H, methyl and halogen;

R¹⁸ is independently selected from H, methyl and halogen;

R¹⁹ is independently selected from C₁₋₄alkyl, OC₁₋₄alkyl, SC₁₋₄ alkyl, and C₃₋₆ cycloalkyl;

R²⁰ is independently selected from H, halogen, CN, and C₁₋₄alkyl;

R²¹ is independently selected from C₁₋₄alkyl, OC₁₋₄alkyl, SC₁₋₄alkyl, and C₃₋₆ cycloalkyl;

R²² is independently selected from C₁₋₄ alkyl and NR^{a}R^{a};

R²⁴, at each occurrence, is independently selected from H, F, Cl, Br, C(═O)NR^{a}R^{a}, C(═O)OR^{b}, —(CH₂)ₙ—OR^{b}, —(CH₂)ₙ—NR^{a}R^{a}, C₁₋₆ alkyl substituted with 0-5 R^{e}, aryl substituted with 0-5 R^{e}, —(CH₂)ₙ—C₃₋₆ cycloalkyl substituted with 0-5 R^{e}, —(CH₂)ₙ—O-4- to 10-membered heterocyclyl substituted with 0-5 R^{e};

R^{a}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}; and —(CH₂)ₙ-heterocyclyl substituted with 0-5 R^{e}; or R^{a} and R^{a} together with the nitrogen atom to which they are both attached form a heterocyclic ring substituted with 0-5 R^{e};

R^{b}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e};

R^{c}, at each occurrence, is independently selected from F, Cl, Br, CN, NO₂, ═O, C₁₋₆ alkyl substituted with 0-5 R^{f}, C₂₋₆ alkenyl, C₂₋₆ alkynyl, —(CH₂)ₙ—C₃₋₆ cycloalkyl, —(CH₂)ₙ-aryl, —(CH₂)ₙ-heterocyclyl, CO₂H, —(CH₂)ₙOR^{f}, SR^{f}, and —(CH₂)ₙNR^{f}R^{f};

R^{f}, at each occurrence, is independently selected from H, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl;

n, at each occurrence, is an integer independently selected from 0, 1, 2, and 4.

In an eighth aspect, the present disclosure provides a compound of Formula (I) or a stereoisomer, a tautomer, a pharmaceutically acceptable salt, or a solvate thereof, within the scope of any of the first, second, third and fourth aspects, wherein:

Ma is (M2-IV):

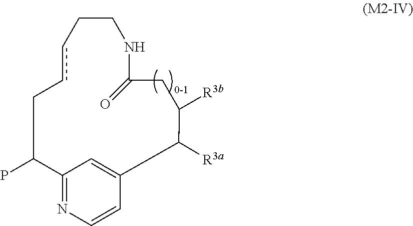

P is independently selected from Formulae (P1), (P2), (P3), (P4), (P5), (P6), (P7), (P8), P(9), P(10), P(11), P(12), P(13), and P(14):

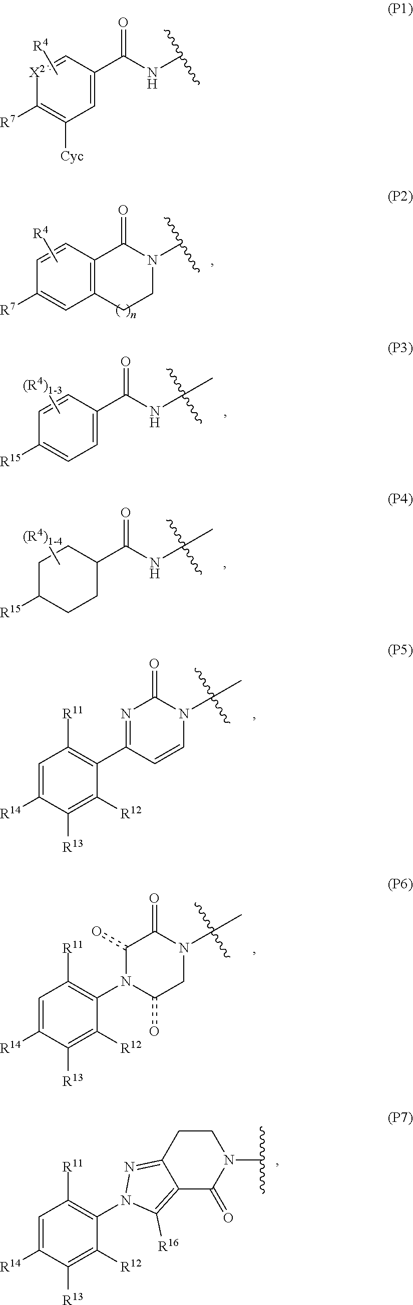

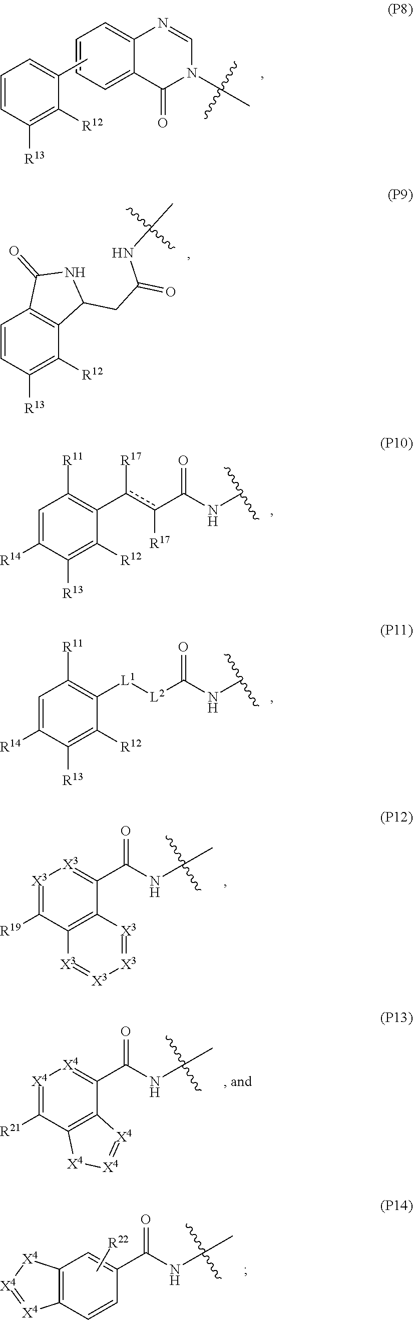

Cyc is C₃₋₆ cycloalkyl;

X² is independently selected from N and CR⁴;

X³ is independently selected from CR¹⁸ and N;

X⁴ is independently selected from CR²⁰, O, S, N, NH, and NC₁₋₄alkyl;

L¹ is independently selected from CH₂, O, NH, NC₁₋₄alkyl, and S;

L² is independently selected from NH and NC₁₋₄alkyl;

R¹ is independently selected from H and C₁₋₄alkyl;

R^{3a} is H;

R^{3b} is H;

alternatively, R^{3a} and R^{3b} are taken together to form

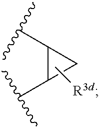

R^{3d} is independently selected from H and ═O;

R⁴ is independently selected from H, halogen, CN, C₁₋₆ alkyl substituted with 1-5 R⁹, and C₃₋₆cycloalkyl substituted with 1-5 R⁹;

R⁵, at each occurrence, is independently selected from H, D, —(CH₂)ₙ—OR^{b}, ═O, —(CH₂)ₙNH₂, —(CH₂)ₙ—CN, halogen, C₁₋₆alkyl, —(CH₂)ₙ—C(═O)OR^{b}, —(CH₂)ₙ—OR^{b}, —(CH₂)ₙ—C₃₋₁₀ carbocyclyl substituted with 0-5 R^{e}, —(CH₂)ₙ-4- to 10-membered heterocyclyl substituted with 0-5 R^{e}, and —O-4- to 10-membered heterocyclyl substituted with 0-5 R^{e};

R⁷ is —(CH₂)ₙNH₂;

R⁹, at each occurrence, is independently selected from H, halogen and OH;

R¹¹ is independently selected from H, halogen, CN, OCH₃, OCF₃, CH₃, C(═O)CH₃, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴;

R¹² is independently selected from H and halogen;

R¹³ is independently selected from H, F, Cl, methyl, ethyl, isopropyl, and OCH₃;

R¹⁴ is independently selected from H, F, and Cl;

R¹⁵ is independently selected from pyrazolyl, aminopyrazolyl, pyrrolidinyl, triazolonyl, aminothiadiazolyl, thiazolyl, pyridinyl, pyrimidinyl, and 2-pyridonyl;

R¹⁶ is independently selected from H, CN, OR^{b}, halogen, NR^{a}R^{a}, and C₁₋₃ alkyl substituted with 0-5 R^{e};

R¹⁷ is independently selected from H, methyl and halogen;

R¹⁸ is independently selected from H, methyl and halogen;

R¹⁹ is independently selected from C₁₋₄alkyl, OC₁₋₄ alkyl, SC₁₋₄alkyl, and C₃₋₆ cycloalkyl;

R²⁰ is independently selected from H, halogen, CN, and C₁₋₄ alkyl;

R²¹ is independently selected from C₁₋₄alkyl, OC₁₋₄ alkyl, SC₁₋₄alkyl, and C₃₋₆ cycloalkyl;

R²² is independently selected from C₁₋₄alkyl and NR^{a}R^{a};

R²⁴, at each occurrence, is independently selected from H, F, Cl, Br. C(═O)NR^{a}R^{a}, C(═O)OR^{b}, —(CH₂)ₙ—OR^{b}, —(CH₂)ₙ—NR^{a}R^{a}, C₁₋₆ alkyl substituted with 0-5 R^{e}, aryl substituted with 0-5 R^{e}, —(CH₂)ₙ—C₃₋₆ cycloalkyl substituted with 0-5 R^{e}, —(CH₂)ₙ—O-4 to 10-membered heterocyclyl substituted with 0-5 R^{e};

R^{a}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ-C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are bath attached form a heterocyclic ring substituted with 0-5 R^{e};

R^{b}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e};

R^{c}, at each occurrence, is independently selected from F, Cl, Br, CN, NO₂, ═O, C₁₋₆ alkyl substituted with 0-5 R^{f}, C₂₋₆ alkenyl, C₂₋₆ alkynyl, —(CH₂)ₙ—C₃₋₆ cycloalkyl, —(CH₂)ₙ-heterocyclyl, CO₂H, —(CH₂)ₙOR^{f}, SR^{f}, and —(CH₂)ₙNR^{f}R^{f}.

R^{f}, at each occurrence, is independently selected from H, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl;

n, at each occurrence, is an integer independently selected from 0, 1, 2, and 4.

In a ninth aspect, the present disclosure provides a compound of Formula (I) or a stereoisomer, a tautomer, a pharmaceutically acceptable salt, or a solvate thereof, within the scope of the first aspect, wherein:

Ma is independently selected from Formula (M3):

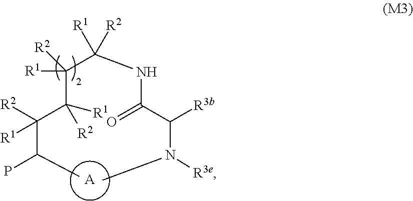

P is independently selected from Formulae (P1), (P2), (P3), (P4), (P5), (P6), (P7), (P8), P(9), P(10), P(11), P(12), P(13), and P(14):

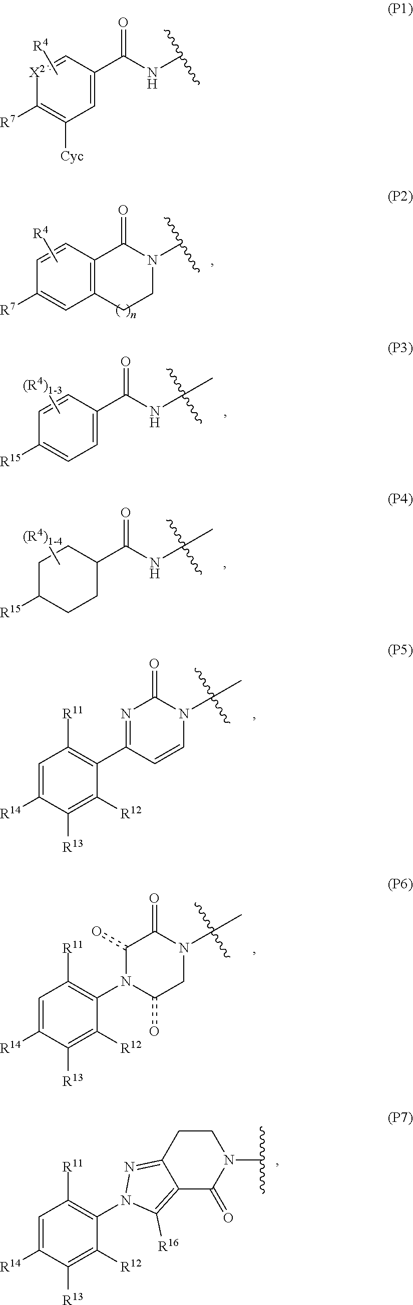

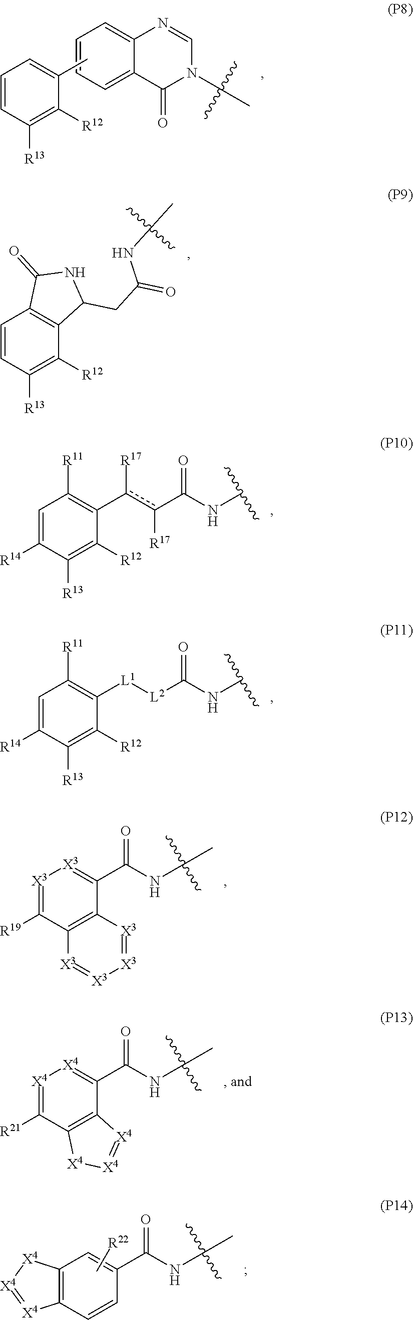

Cyc is C₃₋₆ cycloalkyl;

ring A is independently selected from

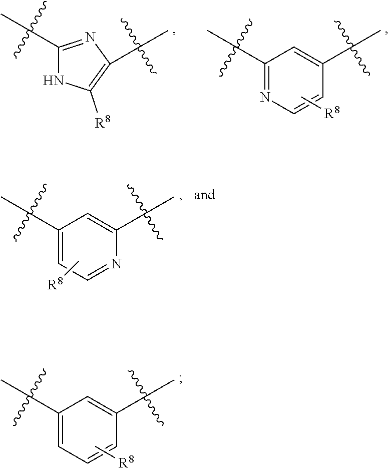

X² is independently selected from N and CR⁴;

X³ is independently selected from CR¹⁸ and N;

X⁴ is independently selected from CR²⁰, O, S, N, NH, and NC₁₋₄alkyl;

L¹ is independently selected from CH₂, O, NH, NC₁₋₄alkyl, and S;

L² is independently selected from NH and NC₁₋₄alkyl;

R¹ and R² are independently selected from H and C₁₋₄alkyl;

alternatively, two adjacent R′ groups form a bond;

R^{3b} is independently selected from —(CH₂)ₙ—NR^{a}R^{a}, —(CH₂)ₙ—C(═O)R^{b}, —(CH₂)ₙ—C(═O)OR^{b}, —(CH₂)ₙ—NR^{a}C(═O)OR^{b}, —(CH₂)ₙNR^{a}C(═O)R^{b}, —NR^{a}C(═O)NR^{a}R^{a}, —C(═O)NR^{a}R^{a};

R^{3c} is independently selected from H and C₁₋₄alkyl;

alternatively, R³b and R^{1c} are taken together to form a heterocyclic ring selected from

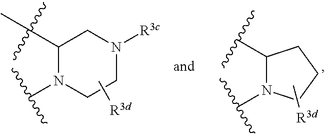

R^{3c} is independently selected from H, C₁₋₄alkyl substituted with 1-5 R⁵, —(CH₂)ₙ—C(═O)R^{b}, —(CH₂)ₙ—C(═O)OR^{b}, —(CH₂)ₙ—C(═O)NR^{a}R^{a}, —(CH₂)ₙ—NHC(═O)OR^{b}, —(CH₂)ₙ—S(═O)ₚR^{c}, —(CH₂)ₙ—S(═O)ₚNR^{a}R^{a}, —(CR_{d}R_{d})ₙ-C₃₋₁₀ carbocyclyl substituted with 1-5 R⁵, and —(CR_{d}R_{d})ₙ-4- to 10-membered heterocyclyl substituted with 1-5 R⁵;

R^{3d} is independently selected from H, ═O, and C₁₋₄alkyl substituted with 1-5 R⁵;

R⁴ is independently selected from H, halogen, CN, C₁₋₆alkyl substituted with 1-5 R⁹, and C₃₋₆ cycloalkyl substituted with 1-5 R⁹;

R⁵, at each occurrence, is independently selected from H, D, —(CH₂)ₙ—OR^{b}, ═O, —(CH₂)ₙNH₂, —(CH₂)ₙCN, halogen, C₁₋₆ alkyl, —(CH₂)ₙ—C(═O)OR^{b}, —(CH₂)ₙ—OR^{b}, —(CH₂)ₙ—C₃₋₁₀ carbocyclyl substituted with 0-5 R^{e}, —(CH₂)ₙ-4- to 10-membered heterocyclyl substituted with 0-5 R^{e}, and —O-4- to 10-membered heterocyclyl substituted with 0-5 R^{e};

R⁷ is —(CH₂)ₙNH₂;

R⁸ is independently selected from H, OH, F, Cl, Br, C₁₋₄ alkyl, C₁₋₄alkoxy, CF₃, CN, C₃₋₆ cycloalkyl, aryl, and 5- to 6-membered heterocycle,

R⁹, at each occurrence, is independently selected from H, halogen and OH;

R¹¹ is independently selected from H, halogen, CN, OCH₃, OCF₃, CH₃, C(═O)CH₃, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴;

R¹² is independently selected from H and halogen;

R¹³ is independently selected from H, F, Cl, methyl, ethyl, isopropyl, and OCH₃;

R¹⁴ is independently selected from H, F, and Cl;

R¹⁵ is independently selected from pyrazolyl, aminopyrazolyl, pyrrolidinyl, triazolonyl, aminothiadiazolyl, oxazolyl, thiazolyl, pyridinyl, pyrimidinyl, and 2-pyridonyl;

R¹⁶ is independently selected from H, CN, OR^{b}, halogen, NR³R^{a}, and C₁₋₃ alkyl substituted with 0-5 R^{e};

R¹⁷ is independently selected from H, methyl and halogen;

R¹⁸ is independently selected from H, methyl and halogen:

R¹⁹ is independently selected from C₁₋₄ alkyl, OC₁₋₄ alkyl, SC₁₋₄alkyl, and C₃₋₆ cycloalkyl;

R²⁰ is independently selected from H, halogen, CN, and C₁₋₄alkyl;

R²¹ is independently selected from C₁₋₄ alkyl, OC₁₋₄ alkyl, SC₁₋₄ alkyl, and C₃₋₆ cycloalkyl;

R²² is independently selected from C₁₋₄alkyl and NR^{a}R^{a};

R²⁴, at each occurrence, is independently selected from H, F, Cl, Br, C(═O)NR^{a}R^{a}, C(═O)OR^{b}, —(CH₂)ₙ—OR^{b}, —(CH₂)ₙ—NR^{a}R^{a}, C₁₋₆ alkyl substituted with 0-5 R^{e}, aryl substituted with 0-5 R^{e}, —(CH₂)ₙ—C₃₋₆ cycloalkyl substituted with 0-5 R^{e}, —(CH₂)ₙ—O-4- to 10-membered heterocyclyl substituted with 0-5 R^{e};

R^{a}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ-C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};

R^{b}, at each occurrence, is independently selected from H, C₁₋₅ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e};

R^{c}, at each occurrence, is independently selected from C₁₋₆ alkyl substituted with 0-5 R^{e}, C₂₋₆alkenyl substituted with 0-5 R^{e}, C₂₋₆alkynyl substituted with 0-5 R^{e}, C₃₋₆carbocyclyl, and heterocyclyl;

R^{d}, at each occurrence, is independently selected from H and C₁₋₄alkyl substituted with 0-5 R^{e};

R^{e}, at each occurrence, is independently selected from F, Cl, Br, CN, NO₂, ═O, C₁₋₆alkyl substituted with 0-5 R^{f}, C₂₋₆ alkenyl, C₂₋₆ alkynyl, —(CH₂)ₙ—C₃₋₆ cycloalkyl, —(CH₂)ₙ-aryl, —(CH₂)ₙ-heterocyclyl, CO₂H, —(CH₂)ₙOR^{f}, SR^{f}, and —(CH₂)ₙNR^{f}R^{f};

R^{f}, at each occurrence, is independently selected from H, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl;

n, at each occurrence, is an integer independently selected from 0, 1. 2, and 4; and

p, at each occurrence, is an integer independently selected from 0, 1, and 2.

In a tenth aspect, the present disclosure provides a compound of Formula (I) or a stereoisomer, a tautomer, a pharmaceutically acceptable salt, or a solvate thereof, within the scope of any of the first and ninth aspects, wherein:

Ma is (M3-I):

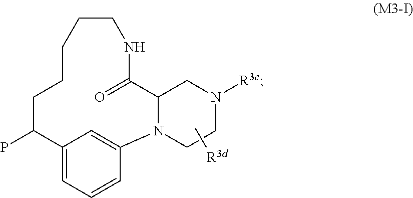

P is independently selected from Formulae (P1), (P2), (P3), (P4), (P5), (P6), (P7), (P8), P(9), P(10), P(11), P(12), P(13), and P(14):

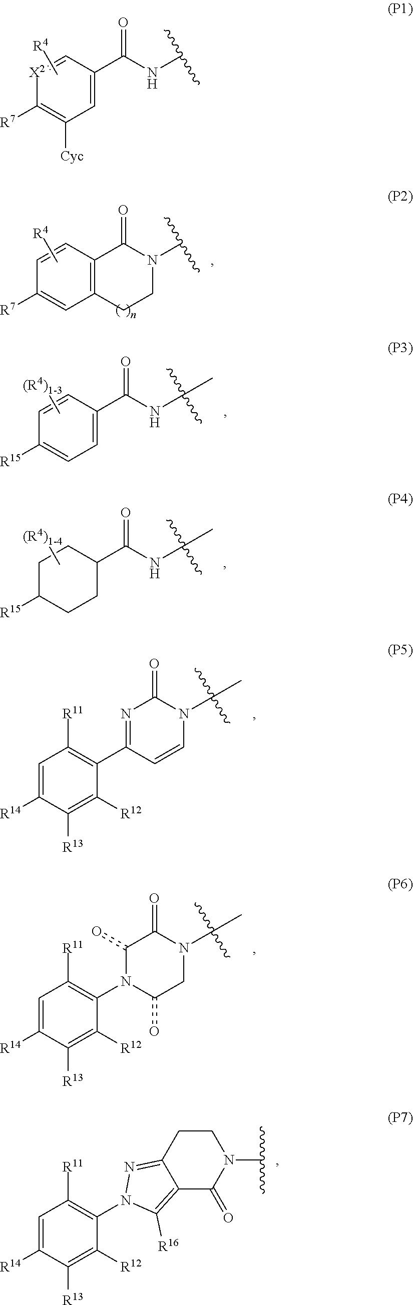

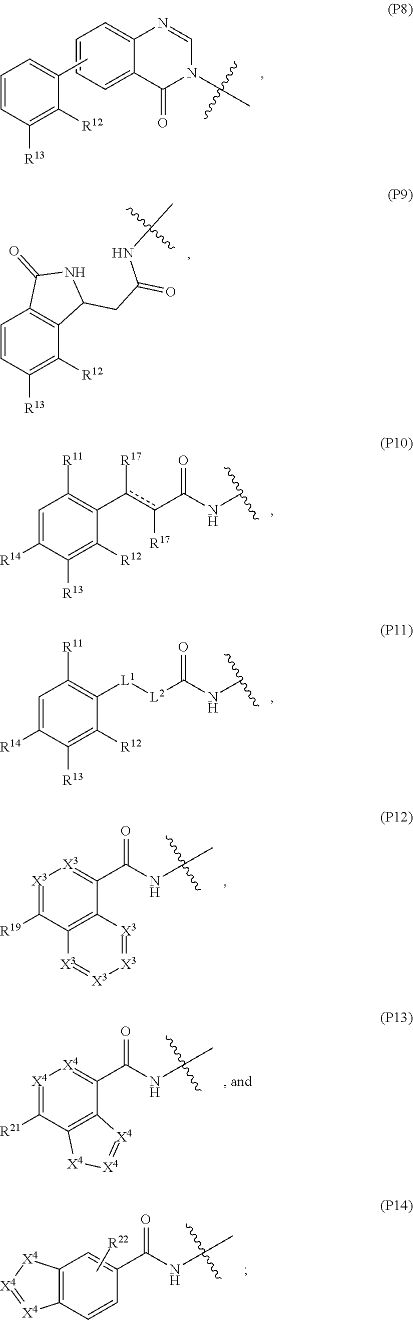

Cyc is C₃₋₆cycloalkyl;

X² is independently selected from N and CR⁴;

X³ is independently selected from CR¹⁸ and N;

X⁴ is independently selected from CR²⁰, O, S, N, NH, and NC₁₋₄alkyl;

L¹ is independently selected from CH₂, O, NH, NC₁₋₄alkyl, and S;

L² is independently selected from NH and NC₁₋₄alkyl;

R^{3c} is independently selected from H, C₁₋₄alkyl, —(CH₂)ₙ—C(═O)R^{b}, —(CH₂)ₙ—C(═O)OR^{b}, —(CH₂)ₙ—C(═O)NR^{a}R^{a}, —(CH₂)ₙ—S(═O)ₚR^{c}, —S(═O)ₚNR^{a}R^{a}, —(CH₂)ₙ—C₃₋₁₀ carbocyclyl substituted with 1-5 R⁵, and —(CR_{d}R_{d})ₙ-4- to 10-membered heterocyclyl substituted with 1-5 R⁵;

R^{3d} is independently selected from H and ═O;

R⁵, at each occurrence, is independently selected from H, D, —(CH₂)ₙ—OR^{b}, ═O, —(CH₂)ₙNH₂, —(CH₂)ₙCN, halogen, C₁₋₆ alkyl, —(CH₂)ₙ—C(═O)OR^{b}, —(CH₂)ₙ—OR^{b}, —(CH₂)ₙ—C₃₋₁₀ carbocyclyl substituted with 0-5 R^{e}. —(CH₂)ₙ-4- to 10-membered heterocyclyl substituted with 0-5 R^{e}, and —O-4- to 10-membered heterocyclyl substituted with 0-5 R^{e};

R⁷ is —(CH₂)ₙNH₂;

R⁸ is independently selected from H, OH, F, Cl, Br, C₁₋₄ alkyl, C₁₋₄ alkoxy, CF₃, CN, C₃₋₆ cycloalkyl, aryl, and 5- to 6-membered heterocycle;

R⁹, at each occurrence, is independently selected from H, halogen and OH;

R¹¹ is independently selected from H, halogen, CN, OCH₃, OCF₃, CH₃, C(═O)CH₃, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴;

R¹² is independently selected from H and halogen;

R¹³ is independently selected from H, F, Cl, methyl, ethyl, isopropyl, and OCH₃;

R¹⁴ is independently selected from H, F, and Cl;

R¹⁵ is independently selected from pyrazolyl, aminopyrazolyl, pyrrolidinyl, triazolonyl, aminothiadiazolyl, oxazolyl, thiazolyl, pyridinyl, pyrimidinyl, and 2-pyridonyl;

R¹⁶ is independently selected from H, CN, OR, halogen, NR^{a}R^{a}, and C₁₋₃ alkyl substituted with 0-5 R^{e};

R¹⁷ is independently selected from H, methyl and halogen;

R¹⁸ is independently selected from H, methyl and halogen;

R¹⁹ is independently selected from C₁₋₄alkyl, OC₁₋₄alkyl, SC₁₋₄alkyl, and C₃₋₆ cycloalkyl;

R²⁰ is independently selected from H, halogen, CN, and C₁₋₄alkyl;

R²¹ is independently selected from C₁₋₄ alkyl, OC₁₋₄ alkyl, SC₁₋₄ alkyl, and C₃₋₆ cycloalkyl;

R²² is independently selected from C₁₋₄alkyl and NR^{a}R^{a};

R²⁴, at each occurrence, is independently selected from H, F, Cl, Br, C(═O)NR^{a}R^{a}, C(═O)OR^{b}, —(CH₂)ₙ—OR^{b}, —(CH₂)ₙ—NR^{a}R^{a}, C₁₋₆ alkyl substituted with 0-5 R^{e}, aryl substituted with 0-5 R^{e}, —(CH₂)ₙ—C₃₋₆ cycloalkyl substituted with 0-5 R^{e}, —(CH₂)ₙ—O-4- to 10-membered heterocyclyl substituted with 0-5 R^{e},

R^{a}, at each occurrence, is independently selected from C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};

R^{b}, at each occurrence, is independently selected from H, C₁₋₆ alkyl substituted with 0-5 R^{e}, C₂ alkenyl substituted with 0-5 R^{e}, C₂₋₆ alkynyl substituted with 0-5 R_{c}, —(CH₂)ₙ—C₃₋₁₀carbocyclyl substituted with 0-5 R^{e}, and —(CH₂)ₙ-heterocyclyl substituted with 0-5 R^{e};

R^{c}, at each occurrence, is independently selected from C₁₋₆ alkyl substituted with 0-5 R^{e}, C₂₋₆alkenyl substituted with 0-5 R^{e}, C₂₋₆alkynyl substituted with 0-5 R^{e}, C₃₋₆carbocyclyl, and heterocyclyl;

R^{d}, at each occurrence, is independently selected from H and C₁₋₄alkyl substituted with 0-5 R^{e};

R^{e}, at each occurrence, is independently selected from F, Cl, Br, CN, NO₂, ═O, C₁₋₆ alkyl substituted with 0-5 R^{f}, C₁₋₆ alkenyl, C₁₋₆ alkynyl, —(CH₂)ₙ—C₃₋₆ cycloalkyl, —(CH₂)ₙ-aryl, —(CH₂)ₙ-heterocyclyl, CO₂H, —(CH₂)ₙOR^{f}, SR^{f}, and —(CH₂)ₙNR^{f}R^{f};

R^{f}, at each occurrence, is independently selected from C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl;

n, at each occurrence, is an integer independently selected from 0, 1, 2, and 4; and

p, at each occurrence, is an integer independently selected from 0, 1, and 2.

In an eleventh aspect, the present disclosure provides a compound of Formula (M4) or a stereoisomer, a tautomer, a pharmaceutically acceptable salt, or a solvate thereof, within the scope of any of the first and ninth aspects, wherein:

Ma is independently selected from Formula (M4):

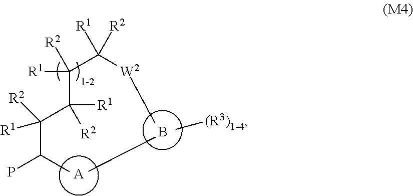

P is independently selected from Formulae (P1), (P2), (P3), (P4). (P5), (P6), (P7), (P8), P(9), P(10), P(11), P(12), P(13), and P(14):

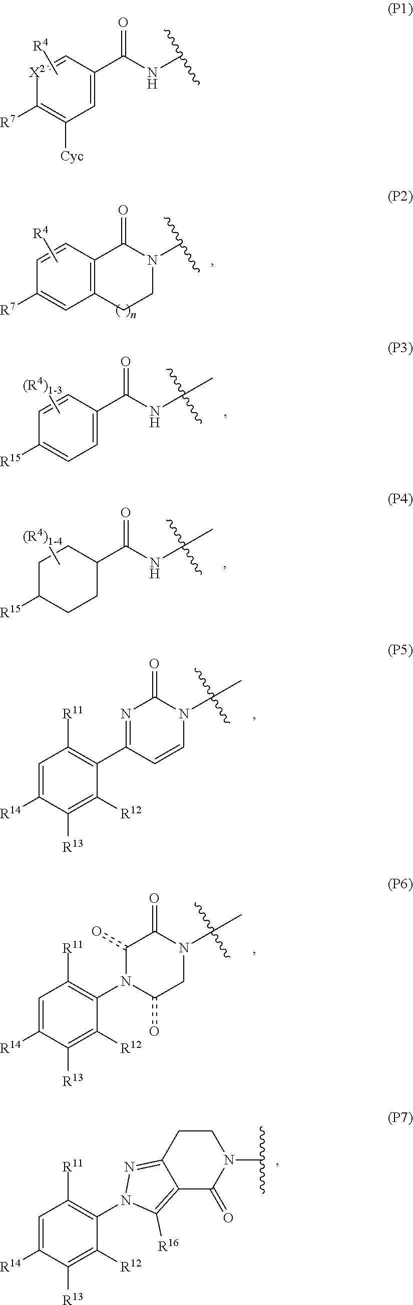

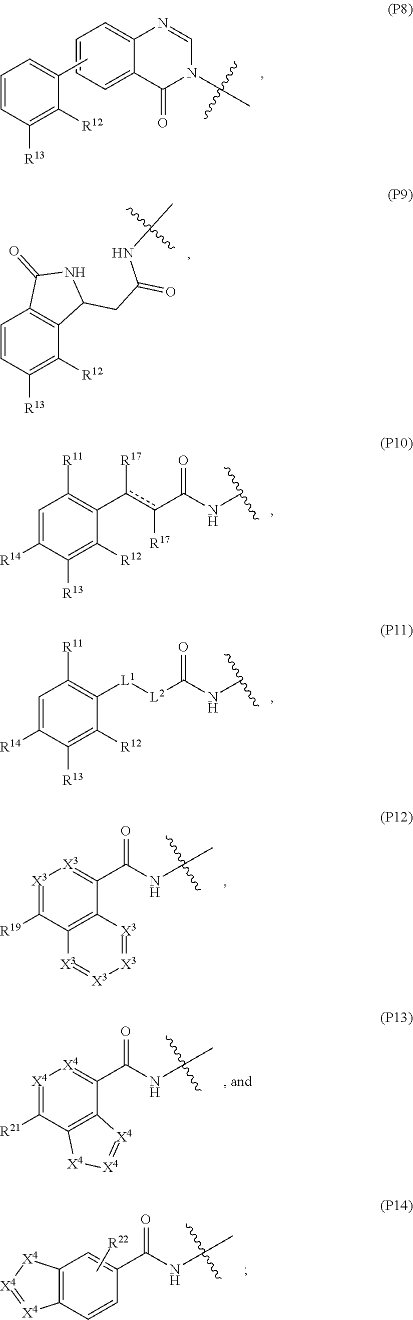

Cyc is C₃₋₆ cycloalkyl; ring A is independently selected from

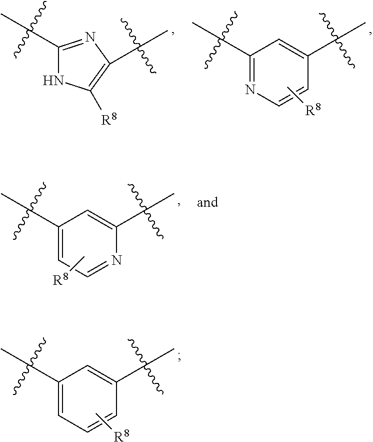

ring B is independently selected from

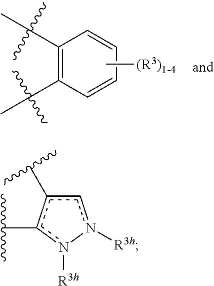

W² is independently selected from —C(O)NH, —NHC(O)—, and —NHC(O)CH₂—;

X² is independently selected from N and CR ⁴;

X³ is independently selected from CR¹⁸ and N;

X⁴ is independently selected from CR²⁰, O, S, N, NH, and NC₁₋₄alkyl;

L¹ is independently selected from CH₂, O, NH, NC₁₋₄alkyl, and S;

L² is independently selected from NH and NC₁₋₄alkyl;

R¹ and R² are independently selected from H and C₁₋₄ alkyl;

R³ is independently selected from H, ═O, halogen, C₁₋₄ alkyl substituted with 1-5 R⁵, CN, —OR^{b}, —NR^{a}R^{a}, —C(═O)R^{b}, —C(═O)OR^{b}, —NR^{a}C(═O)OR^{b}, —NR^{a}C(═O)R^{b}, —NR^{a}C(═O)NR^{a}R^{a}, —C(═O)NR^{a}R^{a}, —S(═O)ₚR^{c}, —(CH₂)ₙ—C₃₋₁₀carbocyclyl substituted with 1-5 R⁵, and —(CH₂)ₙ-4- to 10-membered heterocyclyl substituted with 1-5 R⁵;

R^{3b} is absent or independently selected from H, C₁₋₄alkyl substituted with 1-4 R⁵, —(CH₂)ₙ—C(═O)R^{b}, —(CH₂)ₙ—C(═O)OR^{b};

R⁴ is independently selected from H, halogen, CN, C₁₋₆ alkyl substituted with 1-5 R⁹, and C₃₋₆ cycloalkyl substituted with 1-5 R⁹;

R⁵, at each occurrence, is independently selected from H, D, —OR^{b}, —NH₂, —CN, halogen, C₁₋₆alkyl, —C(═O)OR^{b}, C₃₋₁₀ carbocyclyl substituted with 0-5 R^{e}, -4- to 10-membered heterocyclyl substituted with 0-5 R^{e}, and —O-4- to 10-membered heterocyclyl substituted with 0-5 R^{e};

R⁷ is —(CH₂)ₙNH₂;

R⁸ is independently selected from H, OH, F, Cl, Br, C₁₋₄ alkyl, C₁₋₄ alkoxy, CF₃, CN, C₃₋₆ cycloalkyl, aryl, and 5- to 6-membered heterocycle;

R⁹, at each occurrence, is independently selected from H, halogen and OH;

R¹¹ is independently selected from H, halogen, CN, OCH₃, OCF₃, CH₃, C(═O)CH₃, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴;

R¹² is independently selected from H and halogen;

R¹³ is independently selected from H, F, Cl, methyl, ethyl, isopropyl, and OCH₃;

R¹⁴ is independently selected from H, F, and Cl;

R¹⁵ is independently selected from pyrazolyl, aminopyrazolyl, pyrrolidinyl, triazolonyl, aminothiadiazolyl, oxazolyl, thiazolyl, pyridinyl, pyrimidinyl, and 2-pyridonyl;

R¹⁶ is independently selected from H, CN, OR^{b}, halogen, NR^{a}R^{a}, and C₁₋₃ alkyl substituted with 0-5 R^{e};

R¹⁷ is independently selected from H, methyl and halogen;

R¹⁸ is independently selected from H, methyl and halogen;

R¹⁹ is independently selected from C₁₋₄alkyl, OC₁₋₄ alkyl, SC₁₋₄ alkyl, and C₃₋₆ cycloalkyl;

R²⁰ is independently selected from H, halogen, CN, and C₁₋₄ alkyl;

R²¹ is independently selected from C₁₋₄alkyl, OC₁₋₄alkyl, SC₁₋₄ alkyl, and C₃₋₆ cycloalkyl;

R²² is independently selected from C₁₋₄alkyl and NR^{a}R^{a};

R²⁴, at each occurrence, is independently selected from H, F, Cl, Br, C(═O)NR^{a}R^{a}, ═O)OR^{b}, —(CH₂)ₙ—OR^{b}, —(CH₂)ₙ—NR^{a}R^{a}, C₁₋₆ alkyl substituted with 0-5 R^{e}, aryl substituted with 0-5 R^{e}, —(CF₂)ₙ—C₃₋₆ cycloalkyl substituted with 0-5 R^{e}, —(CH₂)ₙ—O-4- to 10-membered heterocyclyl substituted with 0-5 R^{e};

R^{a}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};

R^{b}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 Rₑ, —(CH₂)ₙ—C₃₋₁₀carbocyclyl substituted with 0-5 R^{e}, and —(CH₂)ₙ-heterocyclyl substituted with 0-5 R^{e};

R^{c}, at each occurrence, is independently selected from C₁₋₆ alkyl substituted with 0-5 R^{e}, C₂₋₆alkenyl substituted with 0-5 R^{e}, C₂₋₆alkynyl substituted with 0-5 R^{e}, C₃₋₆carbocyclyl, and heterocyclyl;

R^{d}, at each occurrence, is independently selected from H and C₁₋₄alkyl substituted with 0-5 R^{e};

R^{e}, at each occurrence, is independently selected from F, Cl, Br, CN, NO₂, ═O, C₁₋₆ alkyl substituted with 0-5 R^{f}, C₂₋₆ alkenyl, C₂₋₆ alkynyl, —(CH₂)ₙ—C₃₋₆ cycloalkyl, —(CH₂)ₙ—heterocyclyl, CO₂H, —(CH₂)ₙOR^{f}, SR^{f}, and —(CH₂)ₙNR^{f}R^{f};

R^{f}, at each occurrence, is independently selected from H, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl;

n, at each occurrence, is an integer independently selected from 0, 1, 2, and 4; and

p, at each occurrence, is an integer independently selected from 0, 1, and 2.

In a twelfth aspect, the present disclosure provides a compound of Formula (I) or a stereoisomer, a tautomer, a pharmaceutically acceptable salt, or a solvate thereof, within the scope of any of the eleventh aspect, wherein:

Ma is (M4-I):

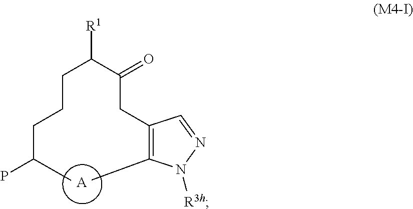

P is independently selected from Formulae (P 1), (P2), (P3), (P4), (P5), (P6), (P7), (P8), P(9). P(10), P(11), P(12), P(13), and P(14):

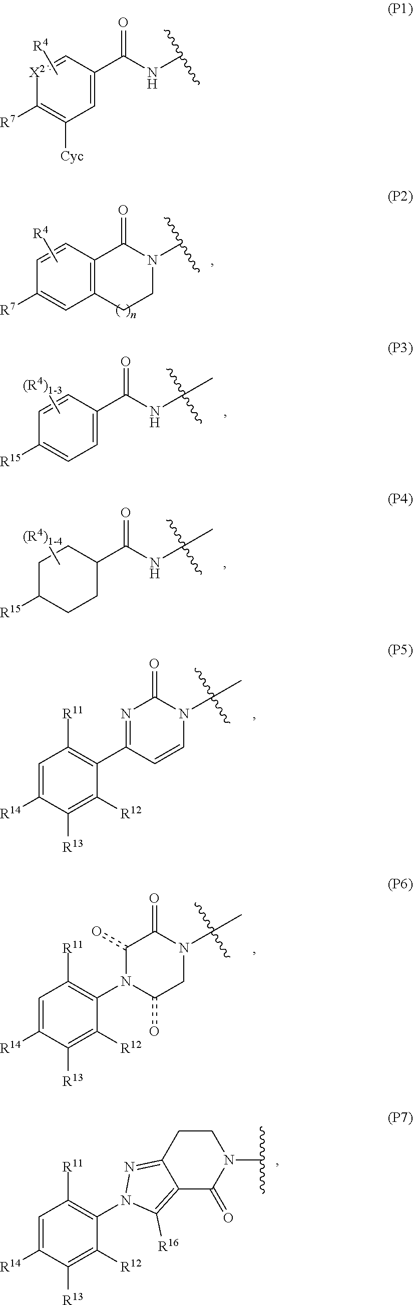

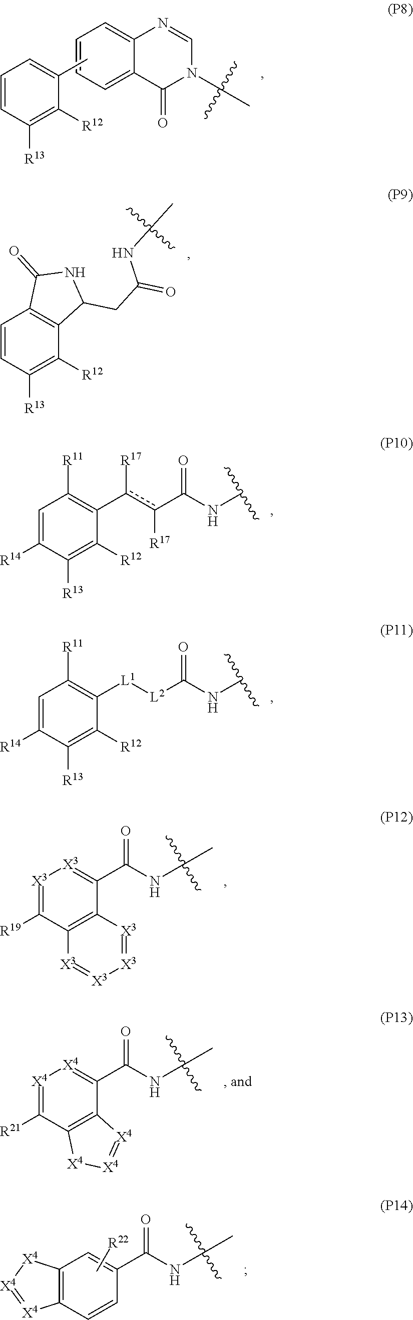

Cyc is C₃₋₆ cycloalkyl;

ring A is independently selected from

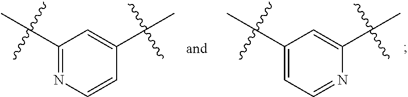 [SMILES: *c1ccnc(*)c1 ; *c1ccnc(*)c1]

X² is independently selected from N mid CR⁴;

X³ is independently selected from CR¹⁸ and N;

X⁴ is independently selected from CR²⁰, O, S, N, NH, and NC₁₋₄alkyl;

L¹ is independently selected from CH₂, O, NH, NC₁₋₄alkyl, and S;

L² is independently selected from NH and NC₁₋₄alkyl;

R¹ is independently selected from H and C₁₋₄alkyl;

R^{3h} is independently selected from H, CHF₂, CD₃, and CH₃;

R⁷ is —(CH₂)ₙNH₂;

R⁹, at each occurrence, is independently selected from H, halogen and OH;

R¹¹ is independently selected from H, halogen, CN, OCH₃, OCF₃, CH₃, C(═O)CH₃, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴;

R¹² is independently selected from H and halogen;

R¹³ is independently selected from H, F, Cl, methyl, ethyl, isopropyl, and OCH₃;

R¹⁴ is independently selected from H, F, and Cl;

R¹⁵ is independently selected from pyrazolyl, aminopyrazolyl, pyrrolidinyl, triazolonyl, aminothiadiazolyl, oxazolyl, thiazolyl, pyridinyl, pyrimidinyl, and 2-pyridonyl;

R¹⁶ is independently selected from H, CN, OR^{b}, halogen, NR^{a}R^{a}, and C₁₋₃ alkyl substituted with 0-5 R^{e};

R¹⁷ is independently selected from H, methyl and halogen;

R¹⁸ is independently selected from H, methyl and halogen;

R¹⁹ is independently selected from C₁₋₄ alkyl, OC₁₋₄ alkyl, SC₁₋₄ alkyl, and C₃₋₆ cycloalkyl;

R²⁰ is independently selected from H, halogen, CN, and C₁₋₄alkyl;

R²¹ is independently selected from C₁₋₄ alkyl, OC₁₋₄ alkyl, SC₁₋₄ alkyl, and C₃₋₆ cycloalkyl;

R²² is independently selected from C₁₋₄alkyl and NR^{a}R^{a};

R²⁴, at each occurrence, is independently selected from H, F, Cl, Br, C(═O)NR^{a}R^{a}, C(═O)OR^{b}, —(CH₂)ₙ—OR^{b}, —(CH₂)ₙ—NR^{a}R^{a}, C₁₋₆ alkyl substituted with 0-5 R^{e}, aryl substituted with 0-5 R^{e}, —(CH₂)ₙ—C₃₋₆ cycloalkyl substituted with 0-5 R^{e}, —(CH₂)ₙ—O-4 to 10-membered heterocyclyl substituted with 0-5 R^{e}.

R^{a}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};

R^{b}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e};

R^{c}, at each occurrence, is independently selected from C₁₋₆ alkyl substituted with 0-5 R^{e}, C₂₋₆alkenyl substituted with 0-5 R^{e}, C₂₋₆alkynyl substituted with 0-5 R^{e}, C₃₋₆carbocyclyl, and heterocyclyl;

R^{d}, at each occurrence, is independently selected from H and C₁₋₄alkyl substituted with 0-5 R^{e};

R^{e}, at each occurrence, is independently selected from F, Cl, Br, CN, NO₂, ═O, C₁₋₆ alkyl substituted with 0-5 R^{f}, C₂₋₆ alkenyl, C₂₋₆ alkynyl, —(CH₂)ₙ—C₂₋₆ cycloalkyl, —(CH₂)ₙ-aryl, —(CH₂)ₙ-heterocyclyl, CO₂H, —(CH₂)ₙOR^{f}, SR^{f}, and —(CH₂)ₙNR^{f}R^{f};

R^{f}, at each occurrence, is independently selected from H, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl;

n, at each occurrence, is an integer independently selected from 0, 1, 2, and 4; and

p, at each occurrence, is an integer independently selected from 0, 1, and 2.

In a thirteenth aspect, the present disclosure provides a compound of Formula (I)

or a stereoisomer, a tautomer, a pharmaceutically acceptable salt, or a solvate thereof, within the scope from the first to the twelfth aspects, wherein:

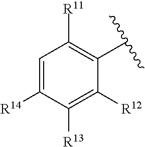

of (P5), (P6), (P7) P(10) and (P11) is independently selected from

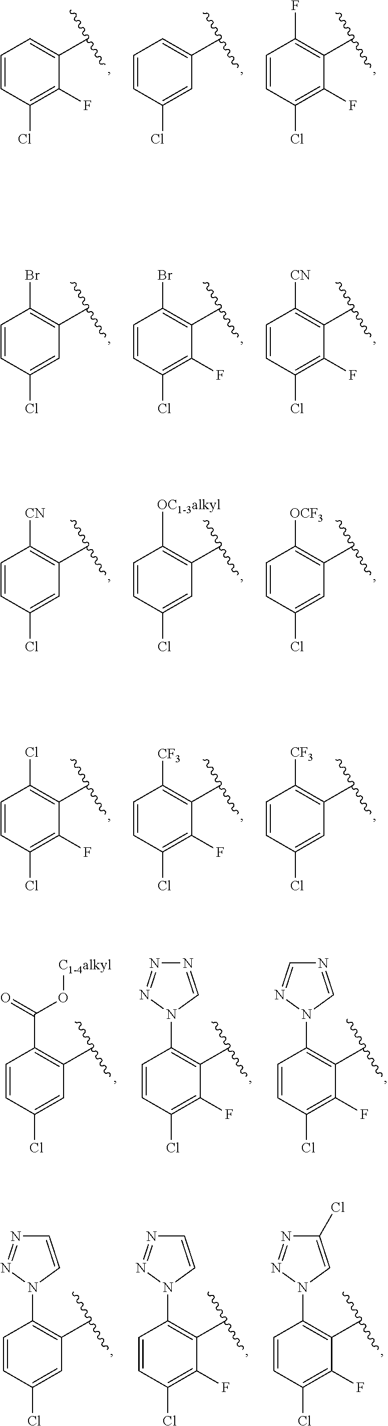

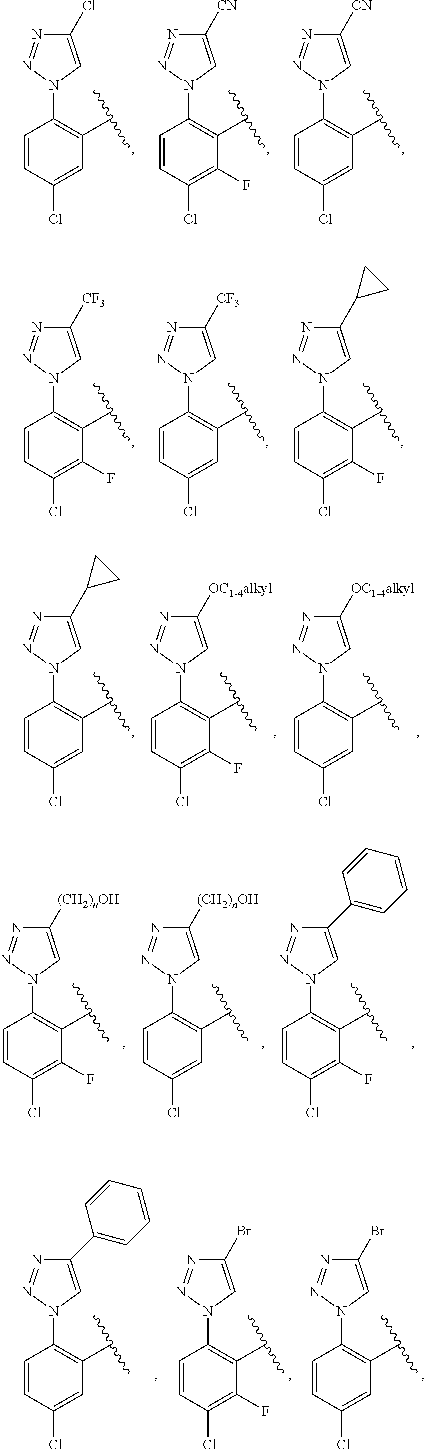

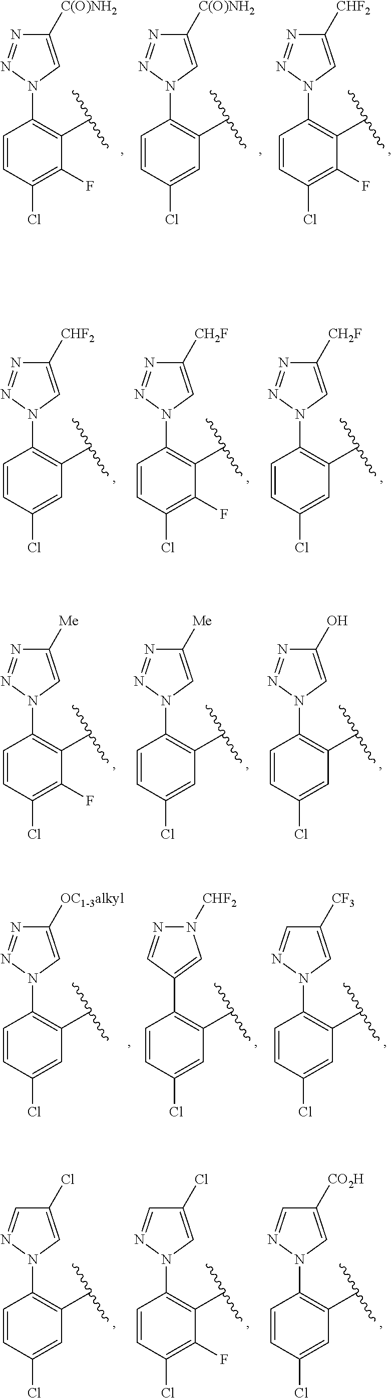

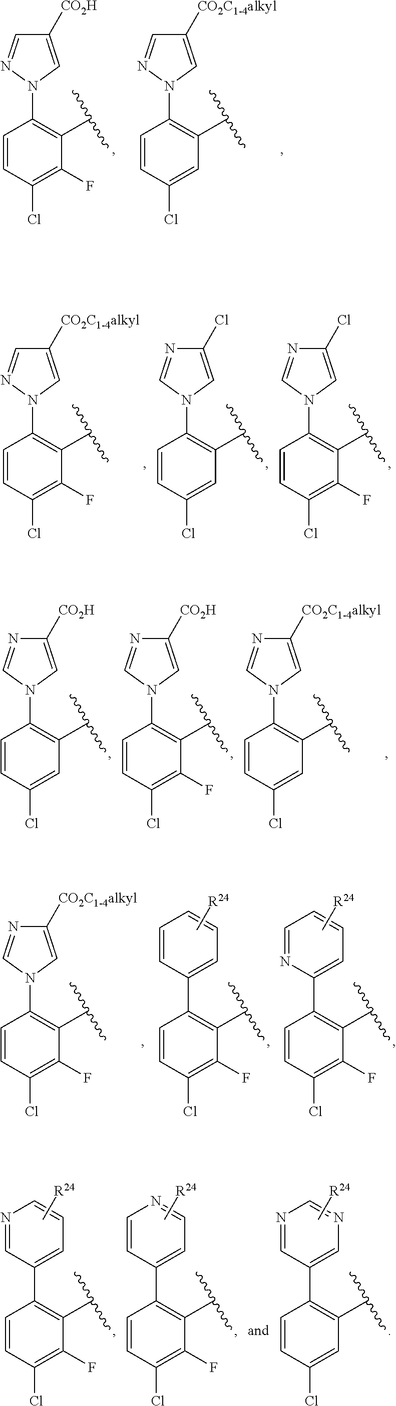

In a fourteenth aspect, the present invention provides a compound selected from the exemplified examples or a stereoisomer, a tautomer, a pharmaceutically acceptable salt, or a solvate thereof.

In another aspect, the present invention provides a compound selected from any subset list of compounds within the scope of the eighth aspect.

In another embodiment, the compounds of the present invention have Factor XIa Kᵢ values ≤10 μM, using the assays disclosed herein, preferably, Kᵢ values ≤1 μM, more preferably, Kᵢ values ≤0.5 μM , even more preferably, Kᵢ values ≤0.1 μM.

In another embodiment, the compounds of the present invention have plasma kallikrein Kᵢ values ≤15 μM, using the assays disclosed herein, preferably, Kᵢ values ≤10 μM, more preferably, Kᵢ values ≤1.0 μM, even more preferably, Kᵢ values ≤0.5 μM.

### II. Other Embodiments of the Invention

In another embodiment, the present invention provides a composition comprising at least one of the compounds of the present invention or a stereoisomer, tautomer, a pharmaceutically acceptable salt, or a solvate thereof.

In another embodiment, the present invention provides a pharmaceutical composition comprising a pharmaceutically acceptable carrier and at least one of the compounds of the present invention or a stereoisomer, a tautomer, a pharmaceutically acceptable salt, or a solvate, thereof.

In another embodiment, the present invention provides a pharmaceutical composition, comprising: a pharmaceutically acceptable carrier and a therapeutically effective amount of at least one of the compounds of the present invention or a stereoisomer, a tautomer, a pharmaceutically acceptable salt, or a solvate thereof.

In another embodiment, the present invention provides a process for making a compound of the present invention.

In another embodiment, the present invention provides an intermediate for making a compound of the present invention.

In another embodiment, the present invention provides a pharmaceutical composition further comprising additional therapeutic agent(s). In a preferred embodiment, the present invention provides pharmace1263utical composition, wherein the additional therapeutic agent(s) are an anti-platelet agent or a combination thereof. Preferably, the anti-platelet agent(s) are clopidogrel and/or aspirin, or a combination thereof.

In another embodiment, the present invention provides a method for the treatment and/or prophylaxis of a thromboembolic disorder comprising administering to a patient in need of such treatment and/or prophylaxis a therapeutically effective amount of at least one of the compounds of the present invention or a stereoisomer, a tautomer, a pharmaceutically acceptable salt, or a solvate thereof.

In another embodiment, the present invention provides a compound of the present invention or a stereoisomer, a tautomer, a pharmaceutically acceptable salt, or a solvate thereof, for use in therapy.

In another embodiment, the present invention provides a compound of the present invention or a stereoisomer, a tautomer, a pharmaceutically acceptable salt, or a solvate thereof, for use in therapy for the treatment and/or prophylaxis of a thromboembolic disorder.

In another embodiment, the present invention also provides the use of a compound of the present invention or a stereoisomer, a tautomer, a pharmaceutically acceptable salt, or a solvate thereof, for the manufacture of a medicament for the treatment and/or prophylaxis of a thromboembolic disorder.

In another embodiment, the present invention provides a method for treatment and/or prophylaxis of a thromboembolic disorder, comprising: administering to a patient in need thereof a therapeutically effective amount of a first and second therapeutic agent, wherein the first therapeutic agent is a compound of the present invention or a stereoisomer, a tautomer, a pharmaceutically acceptable salt, or a solvate thereof, and the second therapeutic agent is at least one agent selected from a factor Xa inhibitor such as apixaban, rivaroxaban, betrixaban, edoxaban, an anti-coagulant agent, an anti-platelet agent, a thrombin inhibiting agent such as dabigatran, a thrombolytic agent, and a fibrinolytic agent. Preferably, the second therapeutic agent is at least one agent selected from warfarin, unfractionated heparin, low molecular weight heparin, synthetic pentasaccharide, hirudin, argatroban, aspirin, ibuprofen, naproxen, sulindac, indomethacin, mefenamate, droxicam, diclofenac, sulfinpyrazone, piroxicam, ticlopidine, clopidogrel, tirofiban, eptifibatide, abciximab, melagatran, desulfatohirudin, tissue plasminogen activator, modified tissue plasminogen activator, anistreplase, urokinase, and streptokinase. Preferably, the second therapeutic agent is at least one anti-platelet agent. Preferably, the anti-platelet agent(s) are clopidogrel and/or aspirin, or a combination thereof.

The thromboembolic disorder includes arterial cardiovascular thromboembolic disorders, venous cardiovascular thromboembolic disorders, arterial cerebrovascular thromboembolic disorders, and venous cerebrovascular thromboembolic disorders. Examples of the thromboembolic disorder include, but are not limited to, unstable angina, an acute coronary syndrome, atrial fibrillation, first myocardial infarction, recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from medical implants, devices, or procedures in which blood is exposed to an artificial surface that promotes thrombosis.

In another embodiment, the present invention provides a method for the treatment and/or prophylaxis of an inflammatory disorder comprising: administering to a patient in need of such treatment and/or prophylaxis a therapeutically effective amount of at least one of the compounds of the present invention or a stereoisomer, a tautomer, a pharmaceutically acceptable salt, or a solvate thereof. Examples of the inflammatory disorder include, but are not limited to, sepsis, acute respiratory distress syndrome, and systemic inflammatory response syndrome.

In another embodiment, the present invention provides a method for the prophylaxis of a disease or condition in which plasma kallikrein activity is implicated comprising administering to a patient in need of such treatment and/or prophylaxis a therapeutically effective amount of at least one of the compounds of the present invention or a stereoisomer, a tautomer, a pharmaceutically acceptable salt, or a solvate thereof.

The disease or condition in which plasma kallikrein activity is implicated includes, but not limited to, impaired visual acuity, diabetic retinopathy, diabetic macular edema, hereditary angioedema, diabetes, pancreatitis, nephropathy, cardio myopathy, neuropathy, inflammatory bowel disease, arthritis, inflammation, septic shock, hypotension, cancer, adult respiratory distress syndrome, disseminated intravascular coagulation, and cardiopulmonary bypass surgery.

In another embodiment, the present invention provides a combined preparation of a compound of the present invention and additional therapeutic agent(s) for simultaneous, separate or sequential use in therapy.

In another embodiment, the present invention provides a combined preparation of a compound of the present invention and additional therapeutic agent(s) for simultaneous, separate or sequential use in treatment and/or prophylaxis of a thromboembolic disorder.

The present invention may be embodied in other specific forms without departing from the spirit or essential attributes thereof. This invention encompasses all combinations of preferred aspects of the invention noted herein. It is understood that any and all embodiments of the present invention may be taken in conjunction with any other embodiment or embodiments to describe additional embodiments. It is also to be understood that each individual element of the embodiments is its own independent embodiment. Furthermore, any element of an embodiment is meant to be combined with any and all other elements from any embodiment to describe an additional embodiment.

### III. Chemistry

Throughout the specification and the appended claims, a given chemical formula or name shall encompass all stereo and optical isomers and racemates thereof where such isomers exist. Unless otherwise indicated, all chiral (enantiomeric and diastereomeric) and racemic forms are within the scope of the invention. Many geometric isomers of C═C double bonds, C═N double bonds, ring systems, and the like can also he present in the compounds, and all such stable isomers are contemplated in the present invention. Cis- and trans- (or E- and Z-) geometric isomers of the compounds of the present invention are described and may be isolated as a mixture of isomers or as separated isomeric forms. The present compounds can be isolated in optically active or racemic forms. Optically active forms may be prepared by resolution of racemic forms or by synthesis from optically active starting materials. All processes used to prepare compounds of the present invention and intermediates made therein are considered to be part of the present invention. When enantiomeric or diastereomeric products are prepared, they may be separated by conventional methods, for example, by chromatography or fractional crystallization. Depending on the process conditions the end products of the present invention are obtained either in free (neutral) or salt form. Both the free form and the salts of these end products are within the scope of the invention. If so desired, one form of a compound may be converted into another form. A free base or acid may be converted into a salt; a salt may be converted into the free compound or another salt; a mixture of isomeric compounds of the present invention may be separated into the individual isomers. Compounds of the present invention, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the invention.

The term “stereoisomer” refers to isomers of identical constitution that differ in the arrangement of their atoms in space. Enantiomers and diastereomers are examples of stereoisomers. The term “enantiomer” refers to one of a pair of molecular species that are minor images of each other and are not superimposable. The term “diastereomer” refers to stereoisomers that are not minor images. The term “racemate” or “racemic mixture” refers to a composition composed of equimolar quantities of two enantiomeric species, wherein the composition is devoid of optical activity.

The symbols “R” and “S” represent the configuration of substituents around a chiral carbon atom(s). The isomeric descriptors “R” and “S” are used as described herein for indicating atom configuration(s) relative to a core molecule and are intended to be used as defined in the literature (IUPAC Recommendations 1996. Pure and Applied Chemistry, 68:2193-2222 (1996)).

The term “chiral” refers to the structural characteristic of a molecule that makes it impossible to superimpose it on its mirror image. The term “homochiral” refers to a state of enantiomeric purity. The term “optical activity” refers to the degree to which a homochiral molecule or nonracemic mixture of chiral molecules rotates a plane of polarized light.

As used herein, the term “alkyl” or “alkylene” is intended to include both branched and straight-chain saturated aliphatic hydrocarbon groups having the specified number of carbon atoms. For example, “C₁ to C₁ alkyl” or “C₁₋₁₀ alkyl” (or alkylene), is intended to include C₁, C₂, C₃, C₄, C₅, C₆, C₇, C₈, C₉, and C₁₀ alkyl groups. Additionally, for example, “C₁ to C₆ alkyl” or “C₁-C₆ alkyl” denotes alkyl having 1 to 6 carbon atoms. Alkyl group can be unsubstituted or substituted with at least one hydrogen being replaced by another chemical group. Example alkyl groups include, but are not limited to, methyl (Me), ethyl (Et), propyl (e.g., n-propyl and isopropyl), butyl (e.g., n-butyl, isobutyl, t-butyl), and pentyl (e.g., n-pentyl, isopentyl, neopentyl). When “C₀ alkyl” or “C₀ alkylene” is used, it is intended to denote a direct bond.

“Alkynyl” or “alkynylene” is intended to include hydrocarbon chains of either straight or branched configuration having one or more, preferably one to three, carbon-carbon triple bonds that may occur in any stable point along the chain. For example, “C₂to C₆ alkynyl” or “C₂₋₆ alkynyl” (or alkynylene), is intended to include C₂, C₃, C₄, C₅, and C₆ alkynyl groups; such as ethynyl, propynyl, butynyl, pentynyl, and hexynyl.

The term “alkoxy” or “alkyloxy” refers to an —O-alkyl group. “C₁ to C₆ alkoxy” or “C₁₋₆ alkoxy” (or alkyloxy), is intended to include C₁, C₂, C₃, C₄, C₅, and C₆ alkoxy groups. Example alkoxy groups include, but are not limited to, methoxy, ethoxy, propoxy (e.g., n-propoxy and isopropoxy), and t-butoxy. Similarly, “alkylthio” or “thioalkoxy” represents an alkyl group as defined above with the indicated number of carbon atoms attached through a sulphur bridge; for example methyl-S— and ethyl-S—.

“Halo” or “halogen” includes fluoro, chloro, bromo, and iodo. “Haloalkyl” is intended to include both branched and straight-chain saturated aliphatic hydrocarbon groups having the specified number of carbon atoms, substituted with 1 or more halogens. Examples of haloalkyl include, but are not limited to, fluoromethyl, difluoromethyl, trifluoromethyl, trichloromethyl, pentafluoroethyl, pentachloroethyl, 2,2,2-trifluoroethyl, heptafluoropropyl, and heptachloropropyl. Examples of haloalkyl also include “fluoroalkyl” that is intended to include both branched and straight-chain saturated aliphatic hydrocarbon groups having the specified number of carbon atoms, substituted with 1 or more fluorine atoms.

“Haloalkoxy” or “haloalkyloxy” represents a haloalkyl group as defined above with the indicated number of carbon atoms attached through an oxygen bridge. For example, “C₁ to C₆ haloalkoxy” or “C₁₋₆ haloalkoxy”, is intended to include C₁, C₂, C₃, C₄, C₅, and C₆ haloalkoxy groups. Examples of haloalkoxy include, but are not limited to, trifluoromethoxy, 2,2,2-trifluoroethoxy, and pentafluorothoxy. Similarly, “haloalkylthio” or “thiohaloalkoxy” represents a haloalkyl group as defined above with the indicated number of carbon atoms attached through a sulphur bridge; for example trifluoromethyl-S—, and pentafluoroethyl-S—.

The term “amino,” as used herein, refers to —NH₂.

The term “substituted amino,” as used herein, refers to the defined terms below having the suffix “amino” such as “acylamino,” “alkylamino,” “arylamino,” etc.

The term “alkoxycarbonyl,” as used herein, refers to an alkoxy group attached to the parent molecular moiety through a carbonyl group.

The term “alkoxycarbonylamino,” as used herein, refers to an —NHR wherein R is an alkoxycarbonyl group.

The term “alkylamino,” as used herein refers to —NHR, wherein R is an alkyl group.

The term “alkylcarbonyl,” as used herein, refers to an alkyl group attached to the parent molecular moiety through a carbonyl group.

The term “alkylcarbonylamino,” as used herein, refers to —NHR wherein R is an alkylcarbonyl group.

The term “aminosulfonyl,” as used herein, refers to —SO₂NH².

The term “arylalkyl,” as used herein, refers to an alkyl group substituted with one, two, or three aryl groups.

The term “arylamino,” as used herein, refers to —NHR wherein R is an aryl group.

The term “arylcarbonyl,” as used herein, refers to an aryl group attached to the parent molecular moiety through a carbonyl group.

The term “arylcarbonylamino,” as used herein refers to —NHR wherein R is an arylcarbonyl group.

The term “carbonyl,” as used herein, refers to —C(O)—.

The term “cyano,” as used herein, refers to —CN.

The term “cycloalkylamino,” as used herein, refers to —NHR wherein R is a cycloalkyl group.

The term “cycloalkylcarbonyl,” as used herein, refers to a cycloalkyl group attached to the parent molecular moiety through a carbonyl group.

The term “cycloalkylcarbonylamino,” as used herein, refers to —NHR wherein R is a cycloalkylcarbonyl group.

The term “cycloalkyloxy,” as used herein, refers to a cycloalkyl group attached to the parent molecular moiety through an oxygen atom.

The term “dialkylamino,” as used herein, refers to NR⁹, wherein each R is an alkyl group. The two alkyl groups are the same or different.

The term “haloalkoxy,” as used herein, refers to a haloalkyl group attached to the parent molecular moiety through an oxygen atom.

The term “haloalkyl,” as used herein, refers to an alkyl group substituted by one, two, three, or four halogen atoms.

The term “haloalkylamino,” as used herein, refers to —NHR wherein R is a haloalkyl group.

The term “carbonyl” refers to C(═O).

The term “carboxy” refers to C(═O)OH.

The term “haloalkylcarbonyl,” as used herein, refers to a haloalkyl group attached to the parent molecular moiety through a carbonyl group.

The term “haloalkylcarbonylamino,” as used herein, refers to —NHR wherein R is a haloalkylcarbonyl group.

The terms “alkylcarbonyl” refer to an alkyl or substituted alkyl bonded to a carbonyl.

The term “alkoxycarbonyl,” as used herein, refers to an alkoxy group attached to the parent molecular moiety through a carbonyl group.

The term “hydroxy” or “hydroxyl” refers to OH.

The term “cycloalkyl” refers to cyclized alkyl groups, including mono-, bi- or poly-cyclic ring systems. “C₃ to C₇ cycloalkyl” or “C₃₋₇ cycloalkyl” is intended to include C₃, C₄, C₅, C₆, and C₇ cycloalkyl groups. Example cycloalkyl groups include, but are not limited to, cyclopropyl, cyclobutyl, cyclopentyl, cyclohexyl, and norbornyl. Branched cycloalkyl groups such as 1-methylcyclopropyl and 2-methylcyclopropyl are included in the definition of “cycloalkyl”.

As used herein, “carbocycle” or “carbocyclic residue” is intended to mean any stable 3-, 4-, 5-, 6-, 7-, or 8-membered monocyclic or bicyclic or 7-, 8-, 9-, 10-, 11-, 12-, or 13-membered bicyclic or tricyclic hydrocarbon ring, any of which may be saturated, partially unsaturated, unsaturated or aromatic. Examples of such carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, [3.3.0]bicyclooctane, [4,3.0]bicyclononane, [4.4.0]bicyclodecane (decalin), [2.2.2]bicyclooctane, fluorenyl, phenyl, naphthyl, indanyl, adamantyl anthracenyl, and tetrahydronaphthyl (tetralin). As shown above, bridged rings are also included in the definition of carbocycle (e.g., [2.2.2]bicyclooctane). Preferred carbocycles, unless otherwise specified, are cyclopropyl, cyclobutyl, cyclopentyl, cyclohexyl, phenyl, and indanyl. When the term “carbocycle” is used, it is intended to include “aryl”. A bridged ring occurs when one or more carbon atoms link two non-adjacent carbon atoms. Preferred bridges are one or two carbon atoms. It is noted that a bridge always converts a monocyclic ring into a tricyclic ring. When a ring is bridged, the substituents recited for the ring may also be present on the bridge.

As used herein, the term “bicyclic carbocycle” or “bicyclic carbocyclic group” is intended to mean a stable 9- or 10-membered carbocyclic ring system that contains two fused rings and consists of carbon atoms. Of the two fused rings, one ring is a benzo ring fused to a second ring; and the second ring is a 5- or 6-membered carbon ring which is saturated, partially unsaturated, or unsaturated. The bicyclic carbocyclic group may be attached to its pendant group at any carbon atom which results in a stable structure. The bicyclic carbocyclic group described herein may be substituted on any carbon if the resulting compound is stable. Examples of a bicyclic carbocyclic group are, but not limited to, naphthyl, 1,2-dihydronaphthyl, 1,2,3,4-tetrahydronaphthyl, and indanyl.

“Aryl” groups refer to monocyclic or polycyclic aromatic hydrocarbons, including, for example, phenyl, naphthyl, and phenanthranyl. Aryl moieties are well known and described, for example, in Hawley's Condensed Chemical Dictionary (13th Ed.), Lewis, R. J, ed., J. Wiley & Sons, Inc., New York (1997). “C₆ or C₁₀aryl” or “C₆₋₁₀ aryl” refers to phenyl and naphthyl. Unless otherwise specified, “aryl”, “C₆ or C₁₀ aryl” or “C₆₋₁₀ aryl” or “aromatic residue” may be unsubstituted or substituted with 1 to 5 groups, preferably 1 to 3 groups, OH, OCH₃, Cl, F, Br, I, CN, NO₂, NH₂, N(CH₃)H, N(CH₃)₂, CF₃, OCF₃, C(═O)CH₃, SCH₃, S(═O)CH₃, S(═O)₂CH₃, CH₃, CH₂CH₃, CO₂H, and CO₂CH₃.

The term “benzyl” as used herein, refers to a methyl group on which one of the hydrogen atoms is replaced by a phenyl group, wherein said phenyl group may optionally be substituted with 1 to 5 groups, preferably 1 to 3 groups, OH, OCH₃, Cl, F, Br, I, CN, NO₂, NH₂, N(CH₃)H, N(CH₃)₂, CF₃, OCF₃, C(═O)CH₃, SCH₃, S(═O)CH₃, S(═O)₂CH₃, CH₃, CH₂CH₃, CO₂H, and CO₂CH₃.

As used herein, the term “heterocycle” or “heterocyclic ring” is intended to mean a stable 3-, 4-, 5-, 6-, or 7-membered monocyclic or bicyclic or 7-, 8-, 9-, 10-, 11-, 12-, 13-, or 14-membered polycyclic heterocyclic ring that is saturated, partially unsaturated, or filly unsaturated, and that contains carbon atoms and 1, 2, 3 or 4 heteroatoms independently selected from the group consisting of N, O and S; and including any polycyclic group in which any of the above-defined heterocyclic rings is fused to a benzene ring. The nitrogen and sulfur heteroatoms may optionally be oxidized. (i.e., N→O and S(O)ₚ, wherein p is 0, 1 or 2). The nitrogen atom may be substituted or unsubstituted N or NR wherein R is H or another substituent, if defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. It is preferred that when the total number of S and O atoms in the heterocycle exceeds 1, then these heteroatoms are not adjacent to one another. It is preferred that the total number of S and O atoms in the heterocycle is not more than 1. When the term “heterocycle” is used, it is intended to include heteroaryl.

Examples of heterocycles include, but are not limited to, acridinyl, azetidinyl, azocinyl, henzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3b]tetrahydrofuran, furanyl, furazanyl, imidazolidinyl, imidazolinyl, imidazolyl, 1H-indazolyl, imidazolopyridinyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isothiazolopyridinyl, isoxazolyl, isoxazolopyridinyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolopyridinyl, oxazolidinylperimidinyl, oxindolyl, pyrimidinyl, phenanthridinyl, phenanthrolinyl, phenazinyl, phenothiazinyl, phenoxathiinyl, phenoxazinyl, phthalazinyl, piperazinyl, piperidinyl piperidonyl, 4-piperidonyl, piperonyl, pteridinyl, purinyl, pyranyl, pyrazinyl, pyrazolidinyl, pyrazolinyl, pyrazolopyridinyl, pyrazolyl, pyridazinyl, pyridooxazolyl, pyridoimidazolyl, pyridothiazolyl, pyridinyl, pyrimidinyl, pyrrolidinyl, pyrrolinyl, 2-pyrrolidonyl, 2H pyrrolyl, pyrrolyl, quinazolinyl, quinolinyl, 4H-quinolizinyl, quinoxalinyl, quinuclidinyl, tetrazolyl, tetrahydrofuranyl, tetrahydroisoquinolinyl, tetrahydroquinolinyl, 6H-1,2,5-thiadiazinyl, 1,2,3-thiadiazolyl, 1 2,4-thiadiazolyl, 1,2,5-thiadiazolyl, 1,3,4-thiadiazolyl, thianthrenyl, thiazolyl, thienyl, thiazolopyridinyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Also included are fused ring and Spiro compounds containing, for example, the above heterocycles.

Examples of 5- to 10-membered heterocycles include, but are not limited to, pyridinyl, furanyl, thienyl, pyrrolyl, pyrazolyl, pyrazinyl, piperazinyl, piperidinyl, imidazolyl, imidazolidinyl, indolyl, tetrazolyl, isoxazolyl, morpholinyl, oxazolyl, oxadiazolyl, oxazolidinyl, tetrahydrofuranyl, thiadiazinyl, thiadiazolyl, thiazolyl, triazinyl, triazolyl, benzimidazolyl, 1H-indazolyl, benzofuranyl, benzothiofuranyl, benztetrazolyl, benzotriazolyl, benzisoxazolyl, benzoxazolyl, oxindolyl, benzoxazolinyl, benzthiazolyl, benzisothiazolyl, isatinoyl, isoquinolinyl, octahydroisoquinolinyl, tetrahydroisoquinolinyl, tetrahydroquinolinyl, isoxazolopyridinyl, quinazolinyl, quinolinyl, isothiazolopyridinyl, thiazolopyridinyl, oxazolopyridinyl, imidazolopyridinyl, and pyrazolopyridinyl.

Examples of 5- to 6-membered heterocycles include, but are not limited to, pyridinyl, furanyl, thienyl, pyrrolyl, pyrazolyl, pyrazinyl, piperazinyl, piperidinyl, imidazolyl, imidazolidinyl, indolyl, tetrazolyl, isoxazolyl, morpholinyl, oxazolyl, oxadiazolyl, oxazolidinyl, tetrahydrofuranyl, thiadiazinyl, thiadiazolyl, thiazolyl, triazinyl, and triazolyl. Also included are fused ring and spiro compounds containing, for example, the above heterocycles.

As used herein, the term “bicyclic heterocycle” or “bicyclic heterocyclic group” is intended to mean a stable 9- or 10-membered heterocyclic ring system which contains two fused rings and consists of carbon atoms and 1, 2, 3, or 4 heteroatoms independently selected from the group consisting of N, O and S. Of the two fused rings, one ring is a 5- or 6-membered monocyclic aromatic ring comprising a 5-membered heteroaryl ring, a 6-membered heteroaryl ring or a benzo ring, each fused to a second ring. The second ring is a 5- or 6-membered monocyclic ring which is saturated, partially unsaturated, or unsaturated, and comprises a 5-membered heterocycle, a 6-membered heterocycle or a carbocycle (provided the first ring is not benzo when the second ring is a carbocycle).

The bicyclic heterocyclic group may be attached to its pendant group at any heteroatom or carbon atom which results in a stable structure. The bicyclic heterocyclic group described herein may be substituted on carbon or on a nitrogen atom if the resulting compound is stable. It is preferred that when the total number of S and O atoms in the heterocycle exceeds 1, then these heteroatoms are not adjacent to one another. It is preferred that the total number of S and O atoms in the heterocycle is not more than 1.

Examples of a bicyclic heterocyclic group are, but not limited to, quinolinyl, isoquinolinyl, phthalazinyl, quinazolinyl, indolyl, isoindolyl, indolinyl, 1H-indazolyl, benzimidazolyl, 1,2,3,4-tetrahydroquinolinyl, 1,2,3,4-tetrahydroisoquinolinyl, 5,6,7,8-tetrahydro-quinolinyl, 2,3-dihydro-benzofuranyl, chromanyl, 1,2,3.4-tetrahydro-quinoxalinyl, and 1,2,3,4-tetrahydro-quinazolinyl,

As used herein, the term “aromatic heterocyclic group” or “heteroaryl” is intended to mean stable monocyclic and polycyclic aromatic hydrocarbons that include at least one heteroatom ring member such as sulfur, oxygen, or nitrogen. Heteroaryl groups include, without limitation, pyridyl, pyrimidinyl, pyrazinyl, pyridazinyl, triazinyl, furyl, quinolyl, isoquinolyl, thienyl, imidazolyl, thiazolyl, indolyl, pyrroyl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, benzodioxolanyl, and benzodioxane. Heteroaryl groups are substituted or unsubstituted. The nitrogen atom is substituted or unsubstituted (i.e., N or NR wherein R is H or another substituent, if defined). The nitrogen and sulfur heteroatoms may optionally be oxidized (i.e., N→O and S(O)ₚ, wherein p is 0, 1 or 2).

Bridged rings are also included in the definition of heterocycle. A bridged ring occurs when one or more atoms (i.e., C, O, N, or S) link two non-adjacent carbon or nitrogen atoms. Examples of bridged rings include, but are not limited to, one carbon atom, two carbon atoms, one nitrogen atom, two nitrogen atoms, and a carbon-nitrogen group. It is noted that a bridge always converts a monocyclic ring into a tricyclic ring. When a ring is bridged, the substituents recited for the ring may also be present on the bridge.

The term “counterion” is used to represent a negatively charged species such as chloride, bromide, hydroxide, acetate, and sulfate.

When a dotted ring is used within a ring structure, this indicates that the ring structure may be saturated, partially saturated or unsaturated.

As referred to herein, the term “substituted” means that at least one hydrogen atom is replaced with a non-hydrogen group, provided that normal valencies are maintained and that the substitution results in a stable compound. When a substituent is keto (i.e., ═O), then 2 hydrogens on the atom are replaced. Keto substituents are not present on aromatic moieties. When a ring system (e.g., carbocyclic or heterocyclic) is said to be substituted with a carbonyl group or a double bond, it is intended that the carbonyl group or double bond be part (i.e., within) of the ring. Ring double bonds, as used herein, are double bonds that are formed between two adjacent ring atoms (e.g., C═C, C═N, or N═N).

In cases wherein there are nitrogen atoms e.g., amines) on compounds of the present invention, these may be converted to N-oxides by treatment with an oxidizing agent (e.g., mCPBA and/or hydrogen peroxides) to afford other compounds of this invention. Thus, shown and claimed nitrogen atoms are considered to cover both the shown nitrogen and its N-oxide (N→O) derivative.

When any variable occurs more than one time in any constituent or formula for a compound, its definition at each occurrence is independent of its definition at every other occurrence. Thus, for example, if a group is shown to be substituted with 0-3 R groups, then said group may optionally be substituted with up to three R groups, and at each occurrence R is selected independently from the definition of R. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom in which such substituent is bonded to the rest of the compound of a given formula, then such substituent may be bonded via any atom in such substituent. Combinations of substituents and/or variables are permissible only if such combinations result in stable compounds.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, and/or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th Edition, Mack Publishing Company, Easton, Pa. (1990), the disclosure of which is hereby incorporated by reference.

In addition, compounds of formula I may have prodrug forms. Any compound that will be converted in vivo to provide the bioactive agent (i.e., a compound of formula I) is a prodrug within the scope and spirit of the invention. Various forms of prodrugs are well known in the art. For examples of such prodrug derivatives, see:

a) Bundgaard, H., ed., Design of Prodrugs, Elsevier (1985), and Widder, K. et al., eds., Methods in Enzymology, 112:309-396, Academic Press (1985);

b) Bundgaard, H., Chapter 5, “Design and Application of Prodrugs.” A Textbook of Drug Design and Development, pp. 113-191, Krosgaard-Larsen, P. et al., eds., Harwood Academic Publishers (1991);

c) Bundgaard. H., Adv. Drug Deliv. Rev., 8:1-38 (1992);

d) Bundgaard, H. et al., J Pharm. Sci., 77:285 (1988); and

e) Kakeya, N. et al., Chem. Pharm. Bull., 32:692 (1984).

Compounds containing a carboxy group can form physiologically hydrolyzable esters that serve as prodrugs by being hydrolyzed in the body to yield formula I compounds per se. Such prodrugs are preferably administered orally since hydrolysis in many instances occurs principally under the influence of the digestive enzymes. Parenteral administration may be used where the ester per se is active, or in those instances where hydrolysis occurs in the blood. Examples of physiologically hydrolyzable esters of compounds of formula I include C₁₋₆alkyl, C₁₋₆alkylbenzyl, 4-methoxyhenzyl, indanyl, phthalyl, methoxymethyl, C₁₋₆alkanoyloxy-C₁₋₆alkyl (e.g., acetoxymethyl, pivaloyloxymethyl or propionyloxymethyl), C₁₋₆alkoxycarbonyloxy-C₁₋₆alkyl (e.g., methoxycarbonyl-oxymethyl or ethoxycarbonyloxymethyl, glycyloxymethyl, phenylglycyloxymethyl, (5-methyl-2-oxo-1,3-dioxolen-4-yl)-methyl), and other well known physiologically hydrolyzable esters used, for example, in the penicillin and cephalosporin arts. Such esters may be prepared by conventional techniques known in the art.

Preparation of prodrugs is well known in the art and described in, for example, King, F. D., ed., Medicinal Chemistry: Principles and Practice, The Royal Society of Chemistry, Cambridge, UK (1994); Testa, B. et al., Hydrolysis in Drug and Prodrug Metabolism, Chemistry, Biochemistry and Enzymology, VCHA and Wiley-VCH Zurich, Switzerland (2003); Wermuth, C. G., ed., The Practice of Medicinal Chemistry, Academic Press, San Diego, Calif. (1999).

The present invention is intended to include all isotopes of atoms occurring in the present compounds. Isotopes include those atoms having the same atomic number but different mass numbers. By way of general example and without limitation, isotopes of hydrogen include deuterium and tritium. Deuterium has one proton and one neutron in its nucleus and that has twice the mass of ordinary hydrogen. Deuterium can be represented by symbols such as “II” or “D”. The term “deuterated” herein, by itself or used to modify a compound or group, refers to replacement of one or more hydrogen atom(s), which is attached to carbon(s), with a deuterium atom. Isotopes of carbon include ¹³C and 14C.

Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds have a variety of potential uses, e.g., as standards and reagents in determining the ability of a potential pharmaceutical compound to bind to target proteins or receptors, or for imaging compounds of this invention bound to biological receptors in vivo or in vitro.

“Stable compound” and “stable structure” are meant to indicate a compound that is sufficiently robust to survive isolation to a useful degree of purity from a reaction mixture, and formulation into an efficacious therapeutic agent. It is preferred that compounds of the present invention do not contain a N-halo, S(O)₂H, or S(O)H group.

The term “solvate” means a physical association of a compound of this invention with one or more solvent molecules, whether organic or inorganic. This physical association includes hydrogen bonding. In certain instances the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution-phase and isolable solvates. Exemplary solvates include, but are not limited to, hydrates, ethanolates, methanolates, and isopropanolates. Methods of solvation are generally known in the art.

Abbreviations as used herein, are defined as follows: “1×” for once, “2×” for twice, “3×” for thrice, “° C.” for degrees Celsius, “eq” for equivalent or equivalents, “g” for gram or grams, “mg” for milligram or milligrams, “L” for liter or liters, “mL” for milliliter or milliliters, “μL” for microliter or microliters, “N” for normal, “M” for molar, “mmol” for millimole or millimoles, “min” for minute or minutes, “h” for hour or hours, “rt” for room temperature, “RT” for retention time, “RBF” for round bottom flask, “atm” for atmosphere, “psi” for pounds per square inch, “conc.” for concentrate, “RCM” for ring-closing metathesis, “sat” or “sat'd ” for saturated, “SFC” for supercritical fluid chromatography “MW” for molecular weight, “trip” for melting point, “ee” for enantiomeric excess, “MS” or “Mass Spec” for mass spectrometry, “ESI” for electrospray ionization mass spectroscopy, “HR” for high resolution, “HRMS” for high resolution mass spectrometry, “LCMS” for liquid chromatography mass spectrometry, “HPLC” for high pressure liquid chromatography, “RP HPLC” for reverse phase HPLC, “TLC” or “tlc” for thin layer chromatography, “NMR” for nuclear magnetic resonance spectroscopy. “nOe” for nuclear Overhauser effect spectroscopy, “¹H” for proton, “δ” for delta, “s” for singlet, “d” for doublet, “t” for triplet, “q” for quartet, “m” for multiplet, “br” for broad, “Hz” for hertz, and “α”, “β”, “R”, “S”, “E”, and “Z” are stereochemical designations familiar to one skilled in the art.
Me methyl
Et ethyl
Pr propyl
i-Pr isopropyl
Bu butyl
i-Bu isobutyl
t-Bu tert-butyl
Ph phenyl
Bn benzyl
Boc or BOC tert-butyloxycarbonyl
Boc₂O di-tert-butyl dicarbonate
AcOH or HOAc acetic acid
AlCl₃ aluminum chloride
AIBN Azobisisobuty ronitrile
BBr₃ boron tribromide
BCl₃ boron trichloride
BEMP 2-tert-butylimino-2-diethylamino-1,3-dimethylperhydro-1,3,2-diazaphosphorine
BOP reagent benzotriazol-1-yloxytris(dimethylamino)phosphonium hexafluorophosphate
Burgess reagent 1-methoxy-N-triethylammoniosulfonyl-methanimidate
Cbz carbobenzyloxy
DCM or CH₂C₁₂ dichloromethane
CH₃CN or ACN acetonitrile
CDCl₃ deutero-chloroform
CHCl₃ chloroform
mCPBA or m-CPBA meta-chloroperbenzoic acid
Cs₂CO₃ cesium carbonate
Cu(OAc)₂ copper (II) acetate
CuI copper(I) iodide
CuSO₄ copper(II) sulfate
Cy₂NMe N-cyclohexyl-N-methylcyclohexanamine
DBU 1,8-diazabicyclo└5.4.0┘undec-7-ene
DCE 1,2 dichloroethane
DEA diethylamine
Dess-Martin 1,1,1-tris(acetyloxy)-1,1-dihydro-1,2-beniziodoxol-3-(1H)-one
DIC or DIPCDI diisopropylcarbodiimide
DIEA, DIPEA or Hunig's base diisopropylethylamine
DMAP 4-dimethylaminopyridine
DME 1,2-dimethoxyethane
DMF dimethyl formamide
DMSO dimethyl sulfoxide
cDNA complimentary DNA
Dppp (R)-(+)-1,2-bis(diphenylphosphino)propane
DuPhos (+)-1,2-bis((2S,5S)-2,5-diethylphospholano)benzene
EDC N-(3-dimethylaminopropyl)-N′-ethylcarbodiimide
EDCI N-(3-dimethylaminopropyl)-N′-ethylcarbodiimide hydrochloride
EDTA ethylenediaminetetraacetic acid
(S,S)-EtDuPhosRh(I) (+)-1,2-bis((2S,5S)-2,5-diethylphospholano)benzene(1,5-cyclooctadiene)rhodium(I) trifluoromethanesulfonate
Et₃N or TEA triethylamine
EtOAc ethyl acetate
Et₂O diethyl ether
EtOH ethanol
GMF glass microfiber filter
Grubbs II (1,3-bis(2,4,6-trimethylphenyl)-2-imidazolidinylidene)dichloro (phenylmethylene)(triycyclohexylphosphine)ruthenium
HCl hydrochloric acid
HATU O-(7-azabenzotriazol-1-yl)-N,N,N′,N′-tetramethyluronium hexafluorophosphate
HEPES 4-(2-hydroxyethyl)piperaxine-1-ethanesulfonic acid
Hex hexane
HOBt or HOBT 1-hydroxybenzotriazole
H₂O₂ hydrogen peroxide
H₂SO₄ sulfuric acid
IBX 2-iodoxybenzoic acid
InCl₃ Indium(III) chloride
Jones reagent CrO₃ in aqueous H₂SO₄, 2 M
K₂CO₃ potassium carbonate
K₂HPO₄ potassium phosphate dibasic
K₃PO₄ potassium phosphate tribasic
KOAc potassium acetate
K₃PO₄ potassium phosphate
LAH lithium aluminum hydride
LG leaving group
LiOH lithium hydroxide
MeOH methanol
MgSO₄ magnesium sulfate
MsOH or MSA methylsulfonic acid
NaCl sodium chloride
NaH sodium hydride
NaHCO₃ sodium bicarbonate
Na₂CO₃ sodium carbonate
NaOH sodium hydroxide
Na₂SO₃ sodium sulfite
Na₂SO₄ sodium sulfate
NBS N-bromosuccinimide
NCS N-chlorosuccinimide
NH₃ ammonia
NH₄Cl ammonium chloride
NH₄OH ammonium hydroxide
NH₄COOH ammonium formate
NMM N-methylmorpholine
OTf triflate or trifluoromethanesulfonate
Pd₂(dba)₃ tris(dibenzylideneacetone)dipalladium(0)
Pd(OAc)₂ palladium(II) acetate
Pd/C palladium on carbon
Pd(dppf)Cl ₂ [1,1′-bis(diphenylphosphino)-ferrocene]dichloropalladium(II)
Ph₃PCl₂ triphenylphosphine dichloride
PG protecting group
POCl₃ phosphorus oxychloride
i-PrOH or IPA isopropanol
PS Polystyrene
rt room temperature
SEM-Cl 2-(trimethysilyl)ethoxymethyl chloride
SiO₂ silica oxide
SnCl₂ tin(II) chloride
TBAI tetra-n-butylammonium iodide
TFA trifluoroacetic acid
THF tetrahydrofuran
TMSCHN₂ trimethylsilyldiazomethane
T3P® propane phosphonic acid anhydride
TRIS tris (hydroxymethyl) aminomethane
pTsOH p-toluenesulfonic acid

The compounds of the present invention can be prepared in a number of ways known to one skilled in the art of organic synthesis, which are described in more detail in Section VI.

### IV. Biology

While blood coagulation is essential to the regulation of an organism's hemostasis, it is also involved in many pathological conditions. In thrombosis, a blood clot, or thrombus, may form and obstruct circulation locally, causing ischemia and organ damage. Alternatively, in a process known as embolism, the clot may dislodge and subsequently become trapped in a distal vessel, where it again causes ischemia and organ damage. Diseases arising from pathological thrombus formation are collectively referred to as thromboembolic disorders and include acute coronary syndrome, unstable angina, myocardial infarction, thrombosis in the cavity of the heart, ischemic stroke, deep vein thrombosis, peripheral occlusive arterial disease, transient ischemic attack, and pulmonary embolism. In addition, thrombosis occurs on artificial surfaces in contact with blood, including catheters, stents, artificial heart valves, and hemodialysis membranes.

Some conditions contribute to the risk of developing thrombosis. For example, alterations of the vessel wall, changes in the flow of blood, and alterations in the composition of the vascular compartment. These risk factors are collectively known as Virchow's triad. (Colman, R. W. et al., eds., Hemostasis and Thrombosis, Basic Principles and Clinical Practice, 5th Edition, p. 853, Lippincott Williams & Wilkins (2006)).

Antithrombotic agents are frequently given to patients at risk of developing thromboembolic disease because of the presence of one or more predisposing risk factors from Virchow's triad to prevent formation of an occlusive thrombus (primary prevention). For example, in an orthopedic surgery setting (e.g., hip and knee replacement), an antithrombotic agent is frequently administered prior to a surgical procedure. The antithrombotic agent counterbalances the prothrombotic stimulus exerted by vascular flow alterations (stasis), potential surgical vessel wall injury, as well as changes in the composition of the blood due to the acute phase response related to surgery. Another example of the use of an antithrombotic agent for primary prevention is dosing with aspirin, a platelet activation inhibitor, in patients at risk for developing thrombotic cardiovascular disease. Well recognized risk factors in this setting include age, male gender, hypertension, diabetes mellitus, lipid alterations, and obesity.

Antithrombotic agents are also indicated for secondary prevention, following an initial thrombotic episode. For example, patients with mutations in factor V (also known as factor V Leiden) and additional risk factors (e.g., pregnancy), are dosed with anticoagulants to prevent the reoccurrence of venous thrombosis. Another example entails secondary prevention of cardiovascular events in patients with a history of acute myocardial infarction or acute coronary syndrome. In a clinical setting, a combination of aspirin and clopidogrel (or other thienopyridines) may be used to prevent a second thrombotic event.

Antithrombotic agents are also given to treat the disease state (i.e., by arresting its development) after it has already started. For example, patients presenting with deep vein thrombosis are treated with anticoagulants (i.e., heparin, warfarin, or LMWH) to prevent further growth of the venous occlusion. Over time, these agents also cause a regression of the disease state because the balance between prothrombotic factors and anticoagulant/profibrinolytic pathways is changed in favor of the latter. Examples on the arterial vascular bed include the treatment of patients with acute myocardial infarction or acute coronary syndrome with aspirin and clopidogrel to prevent further growth of vascular occlusions and eventually leading to a regression of thrombotic occlusions.

Thus, antithrombotic agents are used widely for primary and secondary prevention (i.e., prophylaxis or risk reduction) of thromboembolic disorders, as well as treatment of an already existing thrombotic process. Drugs that inhibit blood coagulation, or anticoagulants, are “pivotal agents for prevention and treatment of thromboembolic disorders” (Hirsh, J. et al., Blood, 105:453-463 (2005)).

An alternative way of initiation of coagulation is operative when blood is exposed to artificial surfaces (e.g., during hemodialysis, “on-pump” cardiovascular surgery, vessel grafts, bacterial sepsis), on cell surfaces, cellular receptors, cell debris, DNA, RNA, and extracellular matrices. This process is also termed contact activation. Surface absorption of factor XII leads to a conformational change in the factor XII molecule, thereby facilitating activation to proteolytic active factor XII molecules (factor XIIa and factor XIIf). Factor XIIa (or XIIf) has a number of target proteins, including plasma prekallikrein and factor XI. Active plasma kallikrein further activates factor XII, leading to an amplification of contact activation. Alternatively, the serine protease prolylcarboxylpeptidase can activate plasma kallikrein complexed with high molecular weight kininogen in a multiprotein complex thrilled on the surface of cells and matrices (Shariat-Madar et al., Blood, 108:192-199 (2006)). Contact activation is a surface mediated process responsible in part for the regulation of thrombosis and inflammation, and is mediated, at least in part, by fibrinolytic-, complement-, kininogen/kinin-, and other humoral and cellular pathways (for review, Coleman. R., “Contact Activation Pathway”, Hemostasis and Thrombosis, pp. 103-122, Lippincott Williams & Wilkins (2001); Schmaier, A. H., “Contact Activation”, Thrombosis and Hemorrhage, pp. 105-128 (1998)). The biological relevance of the contact activation system for thromboembolic diseases is supported by the phenotype of factor XII deficient mice. More specifically, factor XII deficient mice were protected from thrombotic vascular occlusion in several thrombosis models as well as stroke models and the phenotype of the XII deficient mice was identical to XI deficient mice (Renne et al., J. Exp. Med., 202:271-281 (2005); Kleinschmitz et al., J Exp. Med., 203:513-518 (2006)). The fact that factor XI is down-stream from factor XIIa, combined with the identical phenotype of the XII and XI deficient mice suggest that the contact activation system could play a major role in factor XI activation in vivo.

Factor XI is a zymogen of a trypsin-like serine protease and is present in plasma at a relatively low concentration. Proteolytic activation at an internal R³⁶⁹-1370 bond yields a heavy chain (369 amino acids) and a light chain (238 amino acids). The latter contains a typical trypsin-like catalytic triad (H413, D464, and S557). Activation of factor XI by thrombin is believed to occur on negatively charged surfaces, most likely on the surface of activated platelets. Platelets contain high affinity (0.8 nM) specific sites (130-500/platelet) for activated factor XI. After activation, factor XIa remains surface bound and recognizes factor IX as its normal macromolecular substrate. (Galiani, D., Trends Cardiovasc. Med., 10:198-204 (2000)).

In addition to the feedback activation mechanisms described above, thrombin activates thrombin activated fibrinolysis inhibitor (TAFI), a plasma carboxypeptidase that cleaves C-terminal lysine and arginine residues on fibrin, reducing the ability of fibrin to enhance tissue-type plasminogen activator (tPA) dependent plasminogen activation. In the presence of antibodies to FXIa, clot lysis can occur more rapidly independent of plasma TAB concentration. (Bouma, B. N. et al., Thromb. Res., 101:329-354 (2001).) Thus, inhibitors of factor XIa are expected to be anticoagulant and profibrinolytic.

Further evidence for the anti-thromboembolic effects of targeting factor XI is derived from mice deficient in factor XI. It has been demonstrated that complete fXI deficiency protected mice from ferric chloride (FeCl₃)-induced carotid artery thrombosis (Rosen et al., Thromb. Haemost, 87:774-777 (2002); Wang et al., J Thromb. Haemost, 3:695-702 (2005)). Also, factor XI deficiency rescues the perinatal lethal phenotype of complete protein C deficiency (Chan et al., Amer. J. Pathology, 158:469-479 (2001)). Furthermore, baboon cross-reactive, function blocking antibodies to human factor XI protect against baboon arterial—venous shunt thrombosis (Gruber et al., Blood, 102:953-955 (2003)). Evidence for an antithrombotic effect of small molecule inhibitors of factor XIa is also disclosed in published U.S. Patent Publication No. 2004/0180855 A1. Taken together, these studies suggest that targeting factor XI will reduce the propensity for thrombotic and thromboembolic diseases.

Genetic evidence indicates that factor XI is not required for normal homeostasis, implying a superior safety profile of the factor XI mechanism compared to competing antithrombotic mechanisms. In contrast to hemophilia A (factor VIII deficiency) or hemophilia B (factor IX deficiency), mutations of the factor XI gene causing factor XI deficiency (hemophilia C) result in only a mild to moderate bleeding diathesis characterized primarily by postoperative or posttraumatic, but rarely spontaneous hemorrhage. Postoperative bleeding occurs mostly in tissue with high concentrations of endogenous fibrinolytic activity (e.g., oral cavity, and urogenital system). The majority of the cases are fortuitously identified by preoperative prolongation of aPTT (intrinsic system) without any prior bleeding history.

The increased safety of inhibition of XIa as an anticoagulation therapy is further supported by the fact that Factor XI knock-out mice, which have no detectable factor XI protein, undergo normal development, and have a normal life span. No evidence for spontaneous bleeding has been noted. The aPTT (intrinsic system) is prolonged in a gene dose-dependent fashion. Interestingly, even after severe stimulation of the coagulation system (tail transection), the bleeding time is not significantly prolonged compared to wild-type and heterozygous litter mates. (Gailani, D., Frontiers in Bioscience, 6:201-207 (2001); Gailani, D. et al., Blood Coagulation and Fibrinolysis, 8:134-144 (1997).) Taken together, these observations suggest that high levels of inhibition of factor XIa should be well tolerated. This is in contrast to gene targeting experiments with other coagulation factors, excluding factor XII.

In vivo activation of factor XI can be determined by complex formation with either C1 inhibitor or alpha 1 antitrypsin. In a study of 50 patients with acute myocardial infarction (AMI), approximately 25% of the patients had values above the upper normal range of the complex ELISA. This study can be viewed as evidence that at least in a subpopulation of patients with AMI, factor XI activation contributes to thrombin formation (Minnema, M. C. et al., Arterioseler. Thromb. Vase. Biol., 20:2489-2493 (2000)), A second study establishes a positive correlation between the extent of coronary arteriosclerosis and factor XIa in complex with alpha I antitrypsin (Murakami, T. et al., Arterioscler. Thromb. Vase. Biol., 15:1107-1113 (1995)). In another study, Factor XI levels above the 90th percentile in patients were associated with a 2.2-fold increased risk for venous thrombosis (Meijers, J. C. M. et al., N. Engl. J. Med., 342:696-701 (2000)).

Also, it is preferred to find new compounds with improved activity in in vitro clotting assays, compared with known serine protease inhibitors, such as the activated partial thromboplastin time (aPTT) or prothrombin time (PT) assay. (for a description of the aPTT and PT assays see, Goodnight, S. H. et al., “Screening Tests of Hemostasis”, Disorders of Thrombosis and Hemostasis: A Clinical Guide, 2nd Edition, pp. 41-51, McGraw-Hill, New York (2001)).

It is also desirable and preferable to find compounds with advantageous and improved characteristics compared with known serine protease inhibitors, in one or more of the following categories that are given as examples, and are not intended to be limiting: (a) pharmacokinetic properties, including oral bioavailability, half life, and clearance; (b) pharmaceutical properties; (c) dosage requirements; (d) factors that decrease blood concentration peak-to-trough characteristics; (e) factors that increase the concentration of active drug at the receptor; (f) factors that decrease the liability for clinical drug-drug interactions; (g) factors that decrease the potential for adverse side-effects, including selectivity versus other biological targets; and (h) factors that improve manufacturing costs or feasibility.

Pre-clinical studies demonstrated significant antithrombotic effects of small molecule factor XIa inhibitors in rabbit and rat model of arterial thrombosis, at doses that preserved hemostasis. (Wong P. C. et al., American Heart Association Scientific Sessions, Abstract No. 6118, Nov. 12-15, 2006; Schumacher, W. et al., Journal of Thrombosis and Haemostasis, 3(Suppl. 1): P1228 (2005); Schumacher, W. A. et al., European Journal of Pharmacology, 167-174 (2007)). Furthermore, it was observed that in vitro prolongation of the aPTT by specific XIa inhibitors is a good predictor of efficacy in our thrombosis models. Thus, the in vitro aPTT test can be used as a surrogate for efficacy in vivo.

As used herein, the term “patient” encompasses all mammalian species.

As used herein, “treating” or “treatment” cover the treatment of a disease-state in a mammal, particularly in a human, and include: (a) inhibiting the disease-state, i.e., arresting it development; and/or (b) relieving the disease-state, i.e., causing regression of the disease state.

As used herein, “prophylaxis” or “prevention” covers the preventive treatment of a subclinical disease-state in a mammal, particularly in a human, aimed at reducing the probability of the occurrence of a clinical disease-state. Patients are selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state.

As used herein, “risk reduction” covers therapies that lower the incidence of development of a clinical disease state. As such, primary and secondary prevention therapies are examples of risk reduction.

“Therapeutically effective amount” is intended to include an amount of a compound of the present invention that is effective when administered alone or in combination to inhibit factor XIa and/or plasma kallikrein and/or to prevent or treat the disorders listed herein. When applied to a combination, the term refers to combined amounts of the active ingredients that result in the preventive or therapeutic effect, whether administered in combination, serially, or simultaneously.

The term “thrombosis”, as used herein, refers to formation or presence of a thrombus (pl. thrombi): clotting within a blood vessel that may cause ischemia or infarction of tissues supplied by the vessel. The term “embolism”, as used herein, refers to sudden blocking of an artery by a clot or foreign material that has been brought to its site of lodgment by the blood current. The term “thromboembolism”, as used herein, refers to obstruction of a blood vessel with thrombotic material carried by the blood stream from the site of origin to plug another vessel. The term “thromboembolic disorders” entails both “thrombotic” and “embolic” disorders (defined above).

The term “thromboembolic disorders” as used herein includes arterial cardiovascular thromboembolic disorders, venous cardiovascular or cerebrovascular thromboembolic disorders, and thromboembolic disorders in the chambers of the heart or in the peripheral circulation. The term “thromboembolic disorders” as used herein also includes specific disorders selected from, but not limited to, unstable angina or other acute coronary syndromes, atrial fibrillation, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from medical implants, devices, or procedures in which blood is exposed to an artificial surface that promotes thrombosis. The medical implants or devices include, but are not limited to: prosthetic valves, artificial valves, indwelling catheters, stents, blood oxygenators, shunts, vascular access ports, ventricular assist devices and artificial hearts or heart chambers, and vessel grafts. The procedures include, but are not limited to: cardiopulmonary bypass, percutaneous coronary intervention, and hemodialysis. In another embodiment, the term “thromboembolic disorders” includes acute coronary syndrome, stroke, deep vein thrombosis, and pulmonary embolism.

In another embodiment, the present invention provides a method for the treatment of a thromboembolic disorder, wherein the thromboembolic disorder is selected from unstable angina, an acute coronary syndrome, atrial fibrillation, myocardial infarction, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from medical implants, devices, or procedures in which blood is exposed to an artificial surface that promotes thrombosis. In another embodiment, the present invention provides a method for the treatment of a thromboembolic disorder, wherein the thromboembolic disorder is selected from acute coronary syndrome, stroke, venous thrombosis, atrial fibrillation, and thrombosis resulting from medical implants and devices.

In another embodiment, the present invention provides a method for the primary prophylaxis of a thromboembolic disorder, wherein the thromboembolic disorder is selected from unstable angina, an acute coronary syndrome, atrial fibrillation, myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from medical implants, devices, or procedures in which blood is exposed to an artificial surface that promotes thrombosis. In another embodiment, the present invention provides a method for the primary prophylaxis of a thromboembolic disorder, wherein the thromboembolic disorder is selected from acute coronary syndrome, stroke, venous thrombosis, and thrombosis resulting from medical implants and devices.

In another embodiment, the present invention provides a method for the secondary prophylaxis of a thromboembolic disorder, wherein the thromboembolic disorder is selected from unstable angina, an acute coronary syndrome, atrial fibrillation, recurrent myocardial infarction, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from medical implants, devices, or procedures in which blood is exposed to an artificial surface that promotes thrombosis. In another embodiment, the present invention provides a method for the secondary prophylaxis of a thromboembolic disorder, wherein the thromboembolic disorder is selected from acute coronary syndrome, stroke, atrial fibrillation and venous thrombosis.

The term “stroke”, as used herein, refers to embolic stroke or atherothrombotic stroke arising from occlusive thrombosis in the carotid communis, carotid interna, or intracerebral arteries.

It is noted that thrombosis includes vessel occlusion (e.g., after a bypass) and reocclusion (e.g., during or after percutaneous transluminal coronary angioplasty). The thromboembolic disorders may result from conditions including but not limited to atherosclerosis, surgery or surgical complications, prolonged immobilization, arterial fibrillation, congenital thrombophilia, cancer, diabetes, effects of medications or hormones, and complications of pregnancy.

Thromboembolic disorders are frequently associated with patients with atherosclerosis. Risk factors for atherosclerosis include but are not limited to male gender, age, hypertension, lipid disorders, and diabetes mellitus. Risk factors for atherosclerosis are at the same time risk factors for complications of atherosclerosis, i.e., thromboembolic disorders.

Similarly, arterial fibrillation is frequently associated with thromboembolic disorders. Risk factors for arterial fibrillation and subsequent thromboembolic disorders include cardiovascular disease, rheumatic heart disease, nonrheumatic mitral valve disease, hypertensive cardiovascular disease, chronic lung disease, and a variety of miscellaneous cardiac abnormalities as well as thyrotoxicosis.

Diabetes mellitus is frequently associated with atherosclerosis and thromboembolic disorders. Risk factors for the more common type 2 include but are not limited to are family history, obesity, physical inactivity, race/ethnicity, previously impaired fasting glucose or glucose tolerance test, history of aestational diabetes mellitus or delivery of a “big baby”, hypertension, low HDL cholesterol, and polycystic ovary syndrome,

Risk factors for congenital thrombophilia include gain of function mutations in coagulation factors or loss of function mutations in the anticoagulant- or fibrinolytic pathways.

Thrombosis has been associated with a variety of tumor types, e.g., pancreatic cancer, breast cancer, brain tumors, lung cancer, ovarian cancer, prostate cancer, gastrointestinal malignancies, and Hodgkins or non-Hodgkins lymphoma. Recent studies suggest that the frequency of cancer in patients with thrombosis reflects the frequency of a particular cancer type in the general population (Levitan. N. et al., Medicine (Baltimore), 78(5):285-291 (1999); Levine M. et al., N. Engl. J Med., 334(10:677-681 (1996); Blom, J. W. et al., JAMA, 293(6):715-722 (2005)). Hence, the most common cancers associated with thrombosis in men are prostate, colorectal, brain, and lung cancer, and in women are breast, ovary, and lung cancer. The observed rate of venous thromboembolism (VTE) in cancer patients is significant. The varying rates of VTE between different tumor types are most likely related to the selection of the patient population. Cancer patients at risk for thrombosis may possess any or all of the following risk factors: (i) the stage of the cancer (i.e., presence of metastases), (ii) the presence of central vein catheters, (iii) surgery and anticancer therapies including chemotherapy, and (iv) hormones and antiangiogenic drugs. Thus, it is common clinical practice to dose patients having advanced tumors with heparin or low molecular heparin to prevent thromboembolic disorders. A number of low molecular heparin preparations have been approved by the FDA for these indications.

There are three main clinical situations when considering the prevention of VTE in a medical cancer patient: (i) the patient is bedridden for prolonged periods of time; (ii) the ambulatory patient is receiving chemotherapy or radiation; and (iii) the patient is with indwelling central vein catheters. Unfractionated heparin (UFH) and low molecular weight heparin (LMWH) are effective antithrombotic agents in cancer patients undergoing surgery. (Mismetti, P. et al., British Journal of Surgery, 88:913-930 (2001).)

### A. in Vitro Assays

The effectiveness of compounds of the present invention as inhibitors of the coagulation Factors XIa, VIIa, IXa, Xa, XIIa, plasma kallikrein or thrombin, can be determined using a relevant purified serine protease, respectively, and an appropriate synthetic substrate. The rate of hydrolysis of the chromogenic or fluorogenic substrate by the relevant serine protease was measured both in the absence and presence of compounds of the present invention. Hydrolysis of the substrate resulted in the release of pNA (para nitroaniline), which was monitored spectrophotometrically by measuring the increase in absorbance at 405 nm, or the release of AMC (amino methylcoumarin), which was monitored spectrofluorometrically by measuring the increase in emission at 460 nm with excitation at 380 nm. A decrease in the rate of absorbance or fluorescence change in the presence of inhibitor is indicative of enzyme inhibition. Such methods are known to one skilled in the art. The results of this assay are expressed as the inhibitory constant, Kᵢ.

Factor XIa determinations were made in 50 mM HEPES buffer at pH 7.4 containing 145 mM NaCl, 5 mM KCl, and 0.1% PEG 8000 (polyethylene glycol; JT Baker or Fisher Scientific). Determinations were made using purified human Factor XIa at a final concentration of 25-200 pM (Haematologic Technologies) and the synthetic substrate S-2366 (pyroGlu-Pro-Arg-pNA; Chromogenix or AnaSpec) at a concentration of 0.0002-0.001 M.

Factor VIIa determinations were made in 0.005 M calcium chloride, 0.15 M sodium chloride, 0.05 M HEPES buffer containing 0.1% PEG 8000 at a pH of 7.5. Determinations were made using purified human Factor VIIa (Haematologic Technologies) or recombinant human Factor VIIa (Novo Nordisk) at a final assay concentration of 0.5-10 nM, recombinant soluble tissue factor at a concentration of 10-40 nM and the synthetic substrate H-D-Ile-Pro-Arg-pNA (S-2288; Chromogenix or BMPM-2; AnaSpec) at a concentration of 0.001-0.0075 M.

Factor IXa determinations were made in 0.005 M calcium chloride, 0.1 M sodium chloride, 0.0000001 M Refludan (Berlex), 0.05 M TRIS base and 0.5% PEG 8000 at a pH of 7.4. Refludan was added to inhibit small amounts of thrombin in the commercial preparations of human Factor IXa. Determinations were made using purified human Factor IXa (Haematologic Technologies) at a final assay concentration of 20-100 nM and the synthetic substrate PCIXA2100-B (CenterChem) or Pefafluor IXa 3688 (H-D-Leu-Ph'Gly-Arg-AMC; CenterChem) at a concentration of 0.0004-0.0005 M.

Factor Xa determinations were made in 0.1 M sodium phosphate buffer at a pH of 7.5 containing 0.2 M sodium chloride and 0.5% PEG 8000. Determinations were made using purified human Factor Xa (Haematologic Technologies) at a final assay concentration of 150-1000 pM and the synthetic substrate S-2222 (Bz-Ile-Glu (gamma-OMe, 50%)-Gly-Arg-pNA; Chromogenix) at a concentration of 0.0002-0.00035 M.

Factor XIIa determinations were made in 0.05 M HEPES buffer at pH 7.4 containing 0 145 M NaCl, 0.05 M KCl, and 0.1% PEG 8000. Determinations were made using purified human Factor XIIa at a final concentration of 4 nM (American Diagnostica) and the synthetic substrate SPECTROZYME® #312 (H-D-CHT-Gly-L-Arg-pNA.2AcOH; American Diagnostics) at a concentration of 0.00015 M.

Plasma kallikrein determinations were made in 0.1 M sodium phosphate buffer at a pH of 7.5 containing 0.1-0.2 M sodium chloride and 0.5% PEG 8000. Determinations were made using purified human plasma kallikrein (Enzyme Research Laboratories) at a final assay concentration of 200 pM and the synthetic substrate S-2302 (H-(D)-Pro-Phe-Arg-pNA, Chromogenix) at a concentration of 0.00008-0.0004 M.

Thrombin determinations were made in 0.1 M sodium phosphate buffer at a pH of 7.5 containing 0.2 M sodium chloride and 0.5% PEG 8000. Determinations were made using purified human alpha thrombin (Haematologic Technologies or Enzyme Research Laboratories) at a final assay concentration of 200-250 pM and the synthetic substrate S-2366 (pyroGlu-Pro-Arg-pNA; Chromogenix or AnaSpec) at a concentration of 0.0002-0.0004 M.

The Michaelis constant, Kₘ, for substrate hydrolysis by each protease, was determined at 25° C. or 37° C. in the absence of inhibitor. Values of Kᵢ were determined by allowing the protease to react with the substrate in the presence of the inhibitor. Reactions were allowed to go for periods of 20-180 minutes (depending on the protease) and the velocities (rate of absorbance or fluorescence change versus time) were measured. The following relationships were used to calculate Kᵢ values:

(Vₘₐₓ*S)/(Kₘ+S);

(vₒ−vₛ)/vₛ=1/(Kᵢ(1+S/Kₘ)) for a competitive inhibitor with one binding site; or

vₛ/vₒ=A+((B−A)/1+((IC₅₀/(I)ⁿ))); and

Kᵢ=IC₅₀/(1+S/Kₘ) for a competitive inhibitor

where:

vₒ is the velocity of the control in the absence of inhibitor;

vₛ is the velocity in the presence of inhibitor;

Vₘₐₓ is the maximum reaction velocity;

I is the concentration of inhibitor;

A is the minimum activity remaining (usually locked at zero);

B is the maximum activity remaining (usually locked at 1.0);

n is the Hill coefficient, a measure of the number and cooperativity of potential inhibitor binding sites;

IC₅₀ is the concentration of inhibitor that produces 50% inhibition under the assay conditions;

Kᵢ is the dissociation constant of the enzyme: inhibitor complex;

S is the concentration of substrate; and

Kₘ is the Michaelis constant for the substrate.

The selectivity of a compound may be evaluated by taking the ratio of the Kᵢ value for a given protease with the Kᵢ value for the protease of interest (i.e., selectivity for FXIa versus protease P=Kᵢ for protease P/Kᵢ for FXIa). Compounds with selectivity ratios >20 are considered selective.

The effectiveness of compounds of the present invention as inhibitors of coagulation can be determined using a standard or modified clotting assay. An increase in the plasma clotting time in the presence of inhibitor is indicative of anticoagulation. Relative clotting time is the clotting time in the presence of an inhibitor divided by the clotting time in the absence of an inhibitor. The results of this assay may be expressed as IC1.5× or IC2×, the inhibitor concentration required to increase the clotting time by 1.5-time or 2-times, respectively, relative to the clotting time in the absence of the inhibitor. The IC1.5× or IC2× is found by linear interpolation from relative clotting time versus inhibitor concentration plots using inhibitor concentration that spans the IC1.5× or IC2×.

Clotting times are determined using citrated normal human plasma as well as plasma obtained from a number of laboratory animal species (e.g., rat or rabbit). A compound is diluted into plasma beginning with a 10 mM DMSO stock solution. The final concentration of DMSO is less than 2%. Plasma clotting assays are performed in an automated coagulation analyzer (Sysinex, Dade-Behring, Ill.). Similarly, clotting times can be determined from laboratory animal species or humans dosed with compounds of the invention.

Activated Partial Thromboplastin Time (aP UI) is determined using ACTIN® (Dade-Behring, Ill.) following the directions in the package insert. Plasma (0.05 mL) is warmed to 37° C. for 1 minute. ACTIN® (0.05 mL) is added to the plasma and incubated for an additional 2 to 5 minutes. Calcium chloride (25 mM, 0.05 mL) is added to the reaction to initiate coagulation. The clotting time is the time in seconds from the moment calcium chloride is added until a clot is detected.

Prothrombin Time (PT) is determined using thromboplastin (Innovin, Dade-Behring, Ill.) following the directions in the package insert. Plasma (0.05 mL) is warmed to 37° C. for 1 minute. Thromboplastin (0.1 mL) is added to the plasma to initiate coagulation. The clotting time is the time in seconds from the moment thromboplastin is added until a clot is detected.

Chymotrypsin determinations were made in 50 HEPES buffer at pH 7.4 containing 145 mM NaCl. 5 mM KCI, and 0.1% PEG 8000 (polyethylene glycol; JT Baker or Fisher Scientific). Determinations were made using purified human chymotrypsin at a final concentration of 0.2-2 nM (Calbiochem) and the synthetic substrate S-2586 (Methoxy-Succinyl-Arg-Pro-Tyr-pNA; Chromogenix) at a concentration of 0.0005-0.005 M.

Trypsin determinations were made in 0.1 M sodium phosphate buffer at a pH of 7.5 containing 0.2 M sodium chloride and 0.5% PEG 8000. Determinations were made using purified human trypsin (Sigma) at a final assay concentration of 0.1-1 nM and the synthetic substrate S-2222 (Bz-Ile-Glu (gamma-OMe, 50%)-Gly-Arg-pNA; Chromogenix) at a concentration of 0.0005-0.005 M.

The Examples disclosed below were tested in the Factor XIa assay described above and found having Factor XIa inhibitory activity. A range of Factor XIa inhibitory activity (Kᵢ values) of ≤10 μM (10000 nM) was observed.

The Examples disclosed below were tested in the Plasma Kallikrein assay described above, with some Examples having both Factor XIa and Plasma Kallikrein inhibitory activity. For those Examples where the Plasma Kallikrein inhibitory activity was observed as Kᵢ values of ≤10 μM (10000 nM), the inhibitory activity is reported.

### B. In Vivo Assays

The effectiveness of compounds of the present invention as antithrombotic agents can be determined using relevant in vivo thrombosis models, including In Vivo Electrically-induced Carotid Artery Thrombosis Models and In Vivo Rabbit Arteriovenous Shunt Thrombosis Models.

a. In Vivo Electrically-Induced Carotid Artery Thrombosis (ECAT) Model

The rabbit ECAT model, described by Wong et al. (J. Pharmacol. Exp. Ther., 295:212-218 (2000)), can be used in this study. Male New Zealand White rabbits are anesthetized with ketamine (50 mg/kg+50 mg/kg/h IM) and xylazine (10 mg/kg+10 mg/kg/h IM). These anesthetics are supplemented as needed. An electromagnetic flow probe is placed on a segment of an isolated carotid artery to monitor blood flow. Test agents or vehicle will be given (i.v., i.p., s.c., or orally) prior to or after the initiation of thrombosis. Drug treatment prior to initiation of thrombosis is used to model the ability of test agents to prevent and reduce the risk of thrombus formation, whereas dosing after initiation is used to model the ability to treat existing thrombotic disease. Thrombus formation is induced by electrical stimulation of the carotid artery for 3 min at 4 mA using an external stainless-steel bipolar electrode. Carotid blood flow is measured continuously over a 90-min period to monitor thrombus-induced occlusion. Total carotid blood flow over 90 min is calculated by the trapezoidal rule. Average carotid flow over 90 min is then determined by converting total carotid blood flow over 90 min to percent of total control carotid blood flow, which would result if control blood flow had been maintained continuously for 90 min. The ED₅₀ (dose that increased average carotid blood flow over 90 min to 50% of the control) of compounds are estimated by a nonlinear least square regression program using the Hill simoid Eₘₐₓ equation (DeltaGraph; SPSS Inc., Chicago, Ill.).

b. In Vivo Rabbit Arteriovenous (AV) Shunt Thrombosis Model

The rabbit AV shunt model, described by Wong et al, (Wong, P. C. et al., J. Pharmacol. Exp. Ther. 292:351-357 (2000)), can be used in this study. Male New Zealand White rabbits are anesthetized with ketamine (50 mg/kg+50 mg/kg/h IM) and xylazine (10 mg/kg+10 mg/kg/h IM). These anesthetics are supplemented as needed. The femoral artery, jugular vein and femoral vein are isolated and catheterized. A saline-filled AV shunt device is connected between the femoral arterial and the femoral venous cannulae. The AV shunt device consists of an outer piece of tygon tubing (length=8 cm; internal diameter=7.9 mm) and an inner piece of tubing (length=2.5 cm; internal diameter=4.8 mm). The AV shunt also contains an 8-cm-long 2-0 silk thread (Ethicon, Somerville, N.J.). Blood flows from the femoral artery via the AV-shunt into the femoral vein. The exposure of flowing blood to a silk thread induces the formation of a significant thrombus. Forty minutes later, the shunt is disconnected and the silk thread covered with thrombus is weighed. Test agents or vehicle will be given (i.v., i.p., s.c., or orally) prior to the opening of the AV shunt. The percentage inhibition of thrombus formation is determined for each treatment group. The ID₅₀ values (dose that produces 50% inhibition of thrombus formation) are estimated by a nonlinear least square regression program using the Hill sigmoid Eₘₐₓ equation (DeltaGraph; SPSS Inc., Chicago, Ill.).

The anti-inflammatory effect of these compounds can be demonstrated in an Evans Blue dye extravasation assay using C1-esterase inhibitor deficient mice. In this model, mice are dosed with a compound of the present invention, Evans Blue dye is injected via the tail vein, and extravasation of the blue dye is determined by spectrophotometric means from tissue extracts.

The ability of the compounds of the current invention to reduce or prevent the systemic inflammatory response syndrome, for example, as observed during on-pump cardiovascular procedures, can be tested in in vitro perfusion systems, or by on-pump surgical procedures in larger mammals, including dogs and baboons. Read-outs to assess the benefit of the compounds of the present invention include for example reduced platelet loss, reduced platelet/white blood cell complexes, reduced neutrophil elastase levels in plasma, reduced activation of complement factors, and reduced activation and/or consumption of contact activation proteins (plasma kallikrein, factor XII, factor XI, high molecular weight kininogen, C1-esterase inhibitors).

The compounds of the present invention may also be useful as inhibitors of additional serine proteases, notably human thrombin, human plasma kallikrein and human plasmin. Because of their inhibitory action, these compounds are indicated for use in the prevention or treatment of physiological reactions, including blood coagulation, fibrinolysis, blood pressure regulation and inflammation, and wound healing catalyzed by the aforesaid class of enzymes. Specifically, the compounds have utility as drugs for the treatment of diseases arising from elevated thrombin activity of the aforementioned serine proteases, such as myocardial infarction, and as reagents used as anticoagulants in the processing of blood to plasma for diagnostic and other commercial purposes.

### V. Pharmaceutical Compositions, Formulations and Combinations

The compounds of this invention can be administered in such oral dosage forms as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. They can be administered alone, but generally will be administered with a pharmaceutical carrier selected on the basis of the chosen route of administration and standard pharmaceutical practice.

The term “pharmaceutical composition” means a composition comprising a compound of the invention in combination with at least one additional pharmaceutically acceptable carrier. A “pharmaceutically acceptable carrier” refers to media generally accepted in the art for the delivery of biologically active agents to animals, in particular, mammals, including, i.e., adjuvant, excipient or vehicle, such as diluents, preserving agents, fillers, flow regulating agents, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Pharmaceutically acceptable carriers are formulated according to a number of factors well within the purview of those of ordinary skill in the art. These include, without limitation: the type and nature of the active agent being formulated; the subject to which the agent-containing composition is to be administered; the intended route of administration of the composition; and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Remington's Pharmaceutical Sciences, 18th Edition (1990).

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress of the thromboembolic disorder.

By way of general guidance, the daily oral dosage of each active ingredient, when used for the indicated effects, will range between about 0.001 to about 1000 mg/kg of body weight, preferably between about 0.01 to about 100 mg/kg of body weight per day, and most preferably between about 0.1 to about 20 mg/kg/day. Intravenously, the most preferred doses will range from about 0.001 to about 10 mg/kg/minute during a constant rate infusion. Compounds of this invention may be administered in a single daily dose, or the total daily dosage may be administered in divided doses of two, three, or four times daily.

Compounds of this invention can also be administered by parenteral administration (e.g., intra-venous, intra-arterial, intramuscularly, or subcutaneously. When administered intra-venous or intra-arterial, the dose can be given continuously or intermittent. Furthermore, formulation can be developed for intramuscularly and subcutaneous delivery that ensure a gradual release of the active pharmaceutical ingredient. In one embodiment, the pharmaceutical composition is a solid formulation, e.g., a spray-dried composition, which may be used as is, or whereto the physician or the patient adds solvents, and/or diluents prior to use.

Compounds of this invention can be administered in intranasal form via topical use of suitable intranasal vehicles, or via transdermal routes, using transdermal skin patches. When administered in the form of a transdermal delivery system, the dosage administration will, of course, be continuous rather than intermittent throughout the dosage regimen.

The compounds are typically administered in admixture with suitable pharmaceutical diluents, excipients, or carriers (collectively referred to herein as pharmaceutical carriers) suitably selected with respect to the intended form of administration, e.g., oral tablets, capsules, elixirs, and syrups, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels. Solid dispersions are also called solid-state dispersions. In some embodiments, any compound described herein is formulated as a spray dried dispersion (SDD). An SDD is a single phase amorphous molecular dispersion of a drug in a polymer matrix. It is a solid solution prepared by dissolving the drug and a polymer in a solvent (e.g., acetone, methanol or the like) and spray drying the solution. The solvent rapidly evaporates from droplets which rapidly solidifies the polymer and drug mixture trapping the drug in amorphous form as an amorphous molecular dispersion.

Dosage forms (pharmaceutical compositions suitable for administration may contain from about 1 milligram to about 1000 milligrams of active ingredient per dosage unit. In these pharmaceutical compositions the active ingredient will ordinarily be present in an amount of about 0.1-95% by weight based on the total weight of the composition.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl-or propyl-paraben, and chlorobutanol.

Suitable pharmaceutical carriers are described in Remington's Pharmaceutical Sciences, Mack Publishing Company, a standard reference text in this field.

Where the compounds of this invention are combined with other anticoagulant agents, for example, a daily dosage may be about 0.1 to about 100 milligrams of the compound of the present invention and about 0.1 to about 100 milligrams per kilogram of patient body weight. For a tablet dosage form, the compounds of this invention generally may be present in an amount of about 5 to about 300 milligrams per dosage unit, and the second anti-coagulant in an amount of about 1 to about 500 milligrams per dosage unit.

Where the compounds of the present invention are administered in combination with an anti-platelet agent, by way of general guidance, typically a daily dosage may be about 0.01 to about 300 milligrams of the compound of the present invention and about 50 to about 150 milligrams of the anti-platelet agent, preferably about 0.1 to about 4 milligrams of the compound of the present invention and about 1 to about 3 milligrams of antiplatelet agents, per kilogram of patient body weight.

Where the compounds of the present invention are administered in combination with thrombolytic agent, typically a daily dosage may be about 0.1 to about 100 milligrams of the compound of the present invention, per kilogram of patient body weight and, in the case of the thrombolytic agents, the usual dosage of the thrombolytic agent when administered alone may be reduced by about 50-80% when administered with a compound of the present invention.

Particularly when provided as a single dosage unit, the potential exists for a chemical interaction between the combined active ingredients. For this reason, when the compound of the present invention and a second therapeutic agent are combined in a single dosage unit they are formulated such that although the active ingredients are combined in a single dosage unit, the physical contact between the active ingredients is minimized (that is, reduced). For example, one active ingredient may be enteric coated. By enteric coating one of the active ingredients, it is possible not only to minimize the contact between the combined active ingredients, but also, it is possible to control the release of one of these components in the gastrointestinal tract such that one of these components is not released in the stomach but rather is released in the intestines. One of the active ingredients may also be coated with a material that affects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low viscosity grade of hydroxypropyl methylcellulose (HPMC) or other appropriate materials as known in the art, in order to further separate the active components. The polymer coating serves to thrill an additional barrier to interaction with the other component.

These as well as other ways of minimizing contact between the components of combination products of the present invention, whether administered in a single dosage thrill or administered in separate forms but at the same time by the same manner, will be readily apparent to those skilled in the art, once armed with the present disclosure.

In another embodiment, the present invention provides a pharmaceutical composition further comprising additional therapeutic agent(s) selected from potassium channel openers, potassium channel blockers, calcium channel blockers, sodium hydrogen exchanger inhibitors, antiarrhythmic agents, antiatherosclerotic agents, anticoagulants, antithrombotic agents, prothrombolytic agents, fibrinogen antagonists, diuretics, antihypertensive agents. ATPase inhibitors, mineralocorticoid receptor antagonists, phospodiesterase inhibitors, antidiabetic agents, anti-inflammatory agents, antioxidants, angiogenesis modulators, antiosteoporosis agents, hormone replacement therapies, hormone receptor modulators, oral contraceptives, antiobesity agents, antidepressants, antianxiety agents, antipsychotic agents, antiproliferative agents, antitumor agents, antiulcer and gastroesophageal reflux disease agents, growth hormone agents and/or growth hormone secretagogues, thyroid mimetics, anti-infective agents, antiviral agents, antibacterial agents, antifungal agents, cholesterol/lipid lowering agents and lipid profile therapies, and agents that mimic ischemic preconditioning and/or myocardial stunning, or a combination thereof.

In another embodiment, the present invention provides a pharmaceutical composition further comprising additional therapeutic agent(s) selected from an anti-arrhythmic agent, an anti-hypertensive agent, an anti-coagulant agent, an anti-platelet agent, a thrombin inhibiting agent, a thrombolytic agent, a fibrinolytic agent, a calcium channel blocker, a potassium channel blocker, a cholesterol/lipid lowering agent, or a combination thereof.

In another embodiment, the present invention provides a pharmaceutical composition further comprising additional therapeutic agent(s) selected from warfarin, unfractionated heparin, low molecular weight heparin, synthetic pentasaccharide, hirudin, argatroban, aspirin, ibuprofen, naproxen, sulindac, indomethacin, mefenamate, dipyridamol, droxicam, diclofenac, sulfinpyrazone, piroxicam, ticlopidine, clopidogrel, tirofiban, eptifibatide, abciximab, melagatran, ximelagatran, disulfatohirudin, tissue plasminogen activator, modified tissue plasminogen activator, anistreplase, urokinase, and streptokinase, or a combination thereof.

In another embodiment, the present invention provides a pharmaceutical composition wherein the additional therapeutic agent is an antihypertensive agent selected from ACE inhibitors, AT-1 receptor antagonists, beta-adrenergic receptor antagonists, ETA receptor antagonists, dual ETA/AT-1 receptor antagonists, renin inhibitors (alliskerin) and vasopepsidase inhibitors, an antiarrythmic agent selected from IKur inhibitors, an anticoagulant selected from thrombin inhibitors, antithrombin-III activators, heparin co-factor II activators, other factor XIa inhibitors, other kallikrein inhibitors, plasminogen activator inhibitor (PAI-1) antagonists, thrombin activatable fibrinolysis inhibitor (TAFI) inhibitors, factor VIIa inhibitors, factor IXa inhibitors, and factor Xa inhibitors, or an antiplatelet agent selected from GPIIb/IIIa blockers, GP Ib/IX blockers, protease activated receptor 1 (PAR-1) antagonists, protease activated receptor4 (PAR-4) antagonists, prostaglandin E2 receptor EP3 antagonists, collagen receptor antagonists, phosphodiesterase-III inhibitors. P2Y₁ receptor antagonists, P2Y₁₂ antagonists, thromboxane receptor antagonists, cyclooxygense-1 inhibitors, and aspirin, or a combination thereof.

In another embodiment, the present invention provides pharmaceutical composition, wherein the additional therapeutic agent(s) are an anti-platelet agent or a combination thereof.

In another embodiment, the present invention provides a pharmaceutical composition, wherein the additional therapeutic agent is the anti-platelet agent clopidogrel.

The compounds of the present invention can be administered alone or in combination with one or more additional therapeutic agents. By “administered in combination” or “combination therapy” it is meant that the compound of the present invention and one or more additional therapeutic agents are administered concurrently to the mammal being treated. When administered in combination, each component may be administered at the same time or sequentially in any order at different points in time. Thus, each component may be administered separately but sufficiently closely in time so as to provide the desired therapeutic effect.

Compounds that can be administered in combination with the compounds of the present invention include, but are not limited to, anticoagulants, anti-thrombin agents, anti-platelet agents, fibrinolytics, hypolipidemic agents, antihypertensive agents, and anti-ischemic agents.

Other anticoagulant agents (or coagulation inhibitory agents) that may be used in combination with the compounds of this invention include warthrin, heparin (either unfractionated heparin or any commercially available low molecular weight heparin, for example LOVENOX®), synthetic pentasaccharide, direct acting thrombin inhibitors including hirudin and argatroban, as well as other factor VIIa inhibitors, factor: IXa inhibitors, factor Xa inhibitors (e.g., ARIXTRA®, apixaban, rivaroxaban, LY-517717, DU-176b, DX-9065a, and those disclosed in WO 98/57951, WO 03/026652, WO 01/047919, and WO 00/076970), factor XIa inhibitors, and inhibitors of activated TAFI and PAI-1 known in the art.

The term anti-platelet agents (or platelet inhibitory agents), as used herein, denotes agents that inhibit platelet function, for example, by inhibiting the aggregation, adhesion or granule-content secretion of platelets. Such agents include, but are not limited to, the various known non-steroidal anti-inflammatory drugs (NSAIDs) such as acetaminophen, aspirin, codeine, diclofenac, droxicam, fentaynl, ibuprofen, indomethacin, ketorolac, mefenamate, morphine, naproxen, phenacetin, piroxicam, sufentanyl, sulfinpyrazone, sulindac, and pharmaceutically acceptable salts or prodrugs thereof. Of the NSAIDs, aspirin (acetylsalicylic acid or ASA) and piroxicam are preferred. Other suitable platelet inhibitory agents include glycoprotein IIb/IIIa antagonists (e.g., tirofiban, eptifibatide, abciximab, and integrelin), thromboxane-A2-receptor antagonists (e.g., ifetroban), thromboxane-A-synthetase inhibitors, phosphodiesterase-III (PDE-III) inhibitors (e.g., dipyridamole, cilostazol), and PDE-V inhibitors (such as sildenafil), protease-activated receptor 1 (PAR-1) antagonists (e.g., E-5555, SCH-530348, SCH-203099, SCH-529153 and SCH-205831), and pharmaceutically acceptable salts or prodrugs thereof.

Other examples of suitable anti-platelet agents for use in combination with the compounds of the present invention, with or without aspirin, are ADP (adenosine diphosphate) receptor antagonists, preferably antagonists of the purinergic receptors P2Y₁ and. P2Y₁₂, with P2Y₁₂ being even more preferred. Preferred P2Y₁₂ receptor antagonists include clopidogrel, ticlopidine, prasugrel, ticagrelor, and cangrelor, and pharmaceutically acceptable salts or prodrugs thereof. Ticlopidine and clopidogrel are also preferred compounds since they are known to be more gentle than aspirin on the gastro-intestinal tract in use. Clopidogrel is an even more preferred agent.

A preferred example is a triple combination of a compound of the present invention, aspirin, and another anti-platelet agent. Preferably, the anti-platelet agent is clopidogrel or prasugrel, more preferably clopidogrel.

The term thrombin inhibitors (or anti-thrombin agents), as used herein, denotes inhibitors of the serine protease thrombin. By inhibiting thrombin, various thrombin-mediated processes, such as thrombin-mediated platelet activation (that is, for example, the aggregation of platelets, and/or the secretion of platelet granule contents including serotonin) and/or fibrin formation are disrupted. A number of thrombin inhibitors are known to one of skill in the art and these inhibitors are contemplated to be used in combination with the present compounds. Such inhibitors include, but are not limited to, boroarginine derivatives, boropeptides, heparins, hirudin, argatroban, dabigatran, AZD-0837, and those disclosed in WO 98/37075 and WO 02/044145, and pharmaceutically acceptable salts and prodrugs thereof. Boroarginine derivatives and boropeptides include N-acetyl and peptide derivatives of boronic acid, such as C-terminal a-aminoboronic acid derivatives of lysine, ornithine, arginine, homoarginine and corresponding isothiouronium analogs thereof. The term hirudin, as used herein, includes suitable derivatives or analogs of hirudin, referred to herein as hirulogs, such as disulfatohirudin.

The term thrombolytic (or fibrinolytic) agents (or thrombolytics or fibrinolytics), as used herein, denotes agents that lyse blood clots (thrombi). Such agents include tissue plasminogen activator (TPA, natural or recombinant) and modified forms thereof, anistreplase, urokinase, streptokinase, tenecteplase (TNK), lanoteplase (nPA), factor VIIa inhibitors, thrombin inhibitors, inhibitors of factors IXa, Xa, and XIa, PAT-1 inhibitors (i.e., inactivators of tissue plasminogen activator inhibitors), inhibitors of activated TAFI, alpha-2-antiplasmin inhibitors, and anisoylated plasminogen streptokinase activator complex, including pharmaceutically acceptable salts or prodrugs thereof. The term anistreplase, as used herein, refers to anisoylated plasminogen streptokinase activator complex, as described, for example, in European Patent Application No. 028,489, the disclosure of which is hereby incorporated herein by reference herein. The term urokinase, as used herein, is intended to denote both dual and single chain urokinase, the latter also being referred to herein as prourokinase.

Examples of suitable cholesterol/lipid lowering agents and lipid profile therapies for use in combination with the compounds of the present invention include HMG-CoA reductase inhibitors (e.g., pravastatin, lovastatin, simvastatin, fluvastatin, atorvastatin, rosuvastatin, and other statins), low-density lipoprotein (LDL) receptor activity modulators (e.g., HOE-402, PCSK9 inhibitors), bile acid sequestrants (e.g., cholestyramine and colestipol), nicotinic acid or derivatives thereof (e.g., NIASPAN®), GPR109B (nicotinic acid receptor) modulators, fenofibric acid derivatives (e.g., gemfibrozil, clofibrate, fenofibrate and benzafibrate) and other peroxisome proliferator-activated receptors (PPAR) alpha modulators. PPARdelta modulators e.g., GW-501516), PPARgamma modulators (e.g., rosiglitazone), compounds that have multiple functionality for modulating the activities of various combinations of PPARalpha, PPARgamma and PPARdelta, probucol or derivatives thereof (e.g., AGI-1067), cholesterol absorption inhibitors and/or Niemann-Pick Cl-like transporter inhibitors (e.g., ezetimibe), cholesterol ester transfer protein inhibitors (e.g., CP-529414), squalene synthase inhibitors and/or squalene epoxidase inhibitors or mixtures thereof, acyl coenzyme A. cholesteryl acyltransferase (ACAT) 1 inhibitors, ACAT2 inhibitors, dual ACAT1/2 inhibitors, ileal bile acid transport inhibitors (or apical sodium co-dependent bile acid transport inhibitors), microsomal triglyceride transfer protein inhibitors, liver-X-receptor (LXR) alpha modulators, TARbeta modulators, LXR dual alpha/beta modulators, FXR modulators, omega 3 fatty acids (e.g., 3-PUFA), plant stanols and/or fatty acid esters of plant stanols (e.g., sitostanol ester used in BENECOL® margarine), endothelial lipase inhibitors, and HDL functional mimetics which activate reverse cholesterol transport (e.g., apoAI derivatives or apoAI peptide mimetics).

The compounds of the present invention are also useful as standard or reference compounds, for example as a quality standard or control, in tests or assays involving the inhibition of thrombin, Factor VIIa, IXa, Xa, XIa, and/or plasma kallikrein. Such compounds may be provided in a commercial kit, for example, for use in pharmaceutical research involving thrombin, Factor VIIa, IXa, Xa, XIa, and/or plasma kallikrein. XIa. For example, a compound of the present invention could be used as a reference in an assay to compare its known activity to a compound with an unknown activity. This would ensure the experimenter that the assay was being performed properly and provide a basis for comparison, especially if the test compound was a derivative of the reference compound. When developing new assays or protocols, compounds according to the present invention could be used to test their effectiveness.

The compounds of the present invention may also be used in diagnostic assays involving thrombin, Factor VIIa, IXa, Xa, XL, and/or plasma kallikrein. For example, the presence of thrombin, Factor VIIa, IXa, Xa XIa, and/or plasma kallikrein in an unknown sample could be determined by addition of the relevant chromogenic substrate, for example S2366 for Factor XIa, to a series of solutions containing test sample and optionally one of the compounds of the present invention. If production of pNA is observed in the solutions containing test sample, but not in the presence of a compound of the present invention, then one would conclude Factor XIa was present.

Extremely potent and selective compounds of the present invention, those having Kᵢ values less than or equal to 0.001 μM against the target protease and greater than or equal to 0.1 μM against the other proteases, may also be used in diagnostic assays involving the quantitation of thrombin, Factor VIIa, IXa, Xa, XIa, and/or plasma kallikrein in serum samples. For example, the amount of Factor XIa in serum samples could be determined by careful titration of protease activity in the presence of the relevant chromogenic substrate, S2366, with a potent Factor XIa inhibitor of the present invention.

The present invention also encompasses an article of manufacture. As used herein, article of manufacture is intended to include, but not be limited to, kits and packages. The article of manufacture of the present invention, comprises: (a) a first container; (b) a pharmaceutical composition located within the first container, wherein the composition, comprises: a first therapeutic agent, comprising: a compound of the present invention or a pharmaceutically acceptable salt form thereof; and, (c) a package insert stating that the pharmaceutical composition can be used for the treatment of a thromboembolic and/or inflammatory disorder (as defined previously). In another embodiment, the package insert states that the pharmaceutical composition can be used in combination (as defined previously) with a second therapeutic agent to treat a thromboembolic and/or inflammatory disorder. The article of manufacture can further comprise: (d) a second container, wherein components (a) and (b) are located within the second container and component (c) is located within or outside of the second container. Located within the first and second containers means that the respective container holds the item within its boundaries.

The first container is a receptacle used to hold a pharmaceutical composition. This container can be for manufacturing, storing, shipping, and/or individual/bulk selling. First container is intended to cover a bottle, jar, vial, flask, syringe, tube (e.g., for a cream preparation), or any other container used to manufacture, hold, store, or distribute a. pharmaceutical product.

The second container is one used to hold the first container and, optionally, the package insert. Examples of the second container include, but are not limited to, boxes (e.g., cardboard or plastic), crates, cartons, bags (e.g., paper or plastic bags), pouches, and sacks. The package insert can be physically attached to the outside of the first container via tape, glue, staple, or another method of attachment, or it can rest inside the second container without any physical means of attachment to the first container. Alternatively, the package insert is located on the outside of the second container. When located on the outside of the second container, it is preferable that the package insert is physically attached via tape, glue, staple, or another method of attachment. Alternatively, it can be adjacent to or touching the outside of the second container without being physically attached.

The package insert is a label, tag, marker, etc. that recites information relating to the pharmaceutical composition located within the first container. The information recited will usually be determined by the regulatory agency governing the area in which the article of manufacture is to be sold (e.g., the United States Food and Drug Administration). Preferably, the package insert specifically recites the indications for which the pharmaceutical composition has been approved. The package insert may he made of any material on which a person can read information contained therein or thereon. Preferably, the package insert is a printable material (e.g., paper, plastic, cardboard, foil, adhesive-backed paper or plastic, etc.) on which the desired information has been formed (e.g., printed or applied).

Other features of the invention will become apparent in the course of the following descriptions of exemplary embodiments that are given for illustration of the invention and are not intended to be limiting thereof. The following Examples have been prepared, isolated and characterized using the methods disclosed herein.

### VI. General Synthesis Including Schemes

The compounds of the present invention may be synthesized by many methods available to those skilled in the art of organic chemistry (Maffrand, J. P. et al., Heterocycles, 16(1):35-37 (1981)). General synthetic schemes for preparing compounds of the present invention are described below. These schemes are illustrative and are not meant to limit the possible techniques one skilled in the art may use to prepare the compounds disclosed herein. Different methods to prepare the compounds of the present invention will be evident to those skilled in the art. Additionally, the various steps in the synthesis may be performed in an alternate sequence in order to give the desired compound or compounds.

Examples of compounds of the present invention prepared by methods described in the general schemes are given in the intermediates and examples section set out hereinafter. Preparation of homochiral examples may be carried out by techniques known to one skilled in the art. For example, homochiral compounds may be prepared by separation of racemic products by chiral phase preparative HPLC. Alternatively, the example compounds may be prepared by methods known to give enantiomerically enriched products. These include, but are not limited to, the incorporation of chiral auxiliary functionalities into racemic intermediates which serve to control the diastereoselectivity of transformations, providing enantio-enriched products upon cleavage of the chiral auxiliary.

The compounds of the present invention can be prepared in a number of ways known to one skilled in the art of organic synthesis. The compounds of the present invention can be synthesized using the methods described below, together with synthetic methods known in the art of synthetic organic chemistry, or by variations thereon as appreciated by those skilled in the art. Preferred methods include, but are not limited to, those described below. The reactions are performed in a solvent or solvent mixture appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the invention.

It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene et al. (Protective Groups in Organic Synthesis, 4th Edition, Wiley-Interscience (2006)).

Representative compounds 1c of this invention can be prepared as described in Scheme 1. Using amide coupling conditions or condensation reactions of P precursors 1b with an appropriately substituted macrocycle amine la provides compounds 1c.

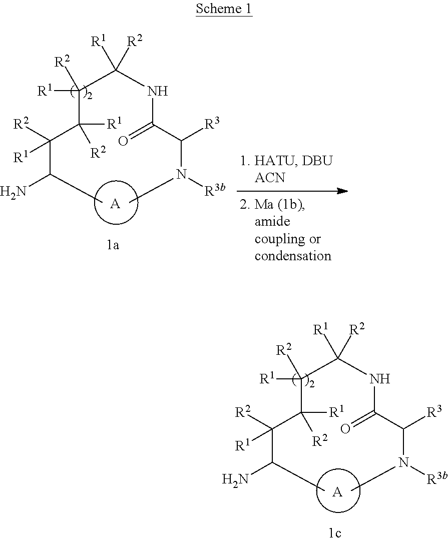

Representative macrocycle amine 2g of this invention can be made as shown Scheme 2. Starting from 2a, coupling with 2b using copper (I) iodide and K₂CO₃ in DMSO to give 2c. Coupling 2c with 2d using T3P to give 2e which goes through GrubbII condition to form macrocycle 4f The double bond and protecting Cbz group can then be taken off under hydrogenation condition to provide macrocycle amine 2g.

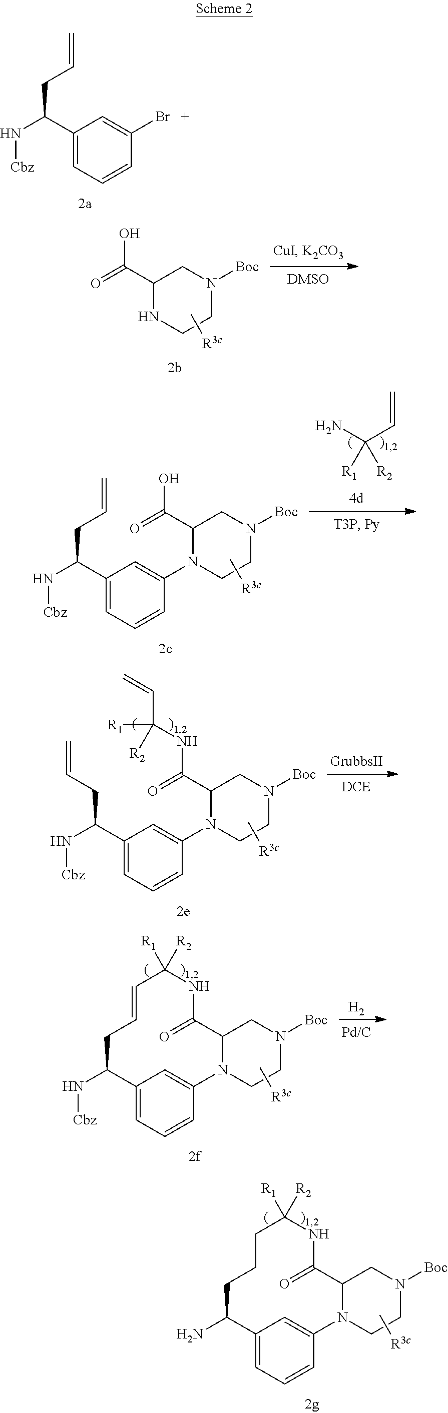

Representative macrocycle amine 3k of this invention can be made as shown in Scheme 3. Starting from 3a, coupling with 3b using copper (1) iodide and CsF in DMSO to give 3c. Basic hydrolysis of 3e afforded 3d. Coupling 3d with 3e using T3P gave 3f which goes through GrubbII reaction conditions to form macrocycle 3g. The double bond and protecting Boc group can then be taken off under hydrogenation, then acidic conditions, followed by free-basing to provide macrocycle amine 3h.

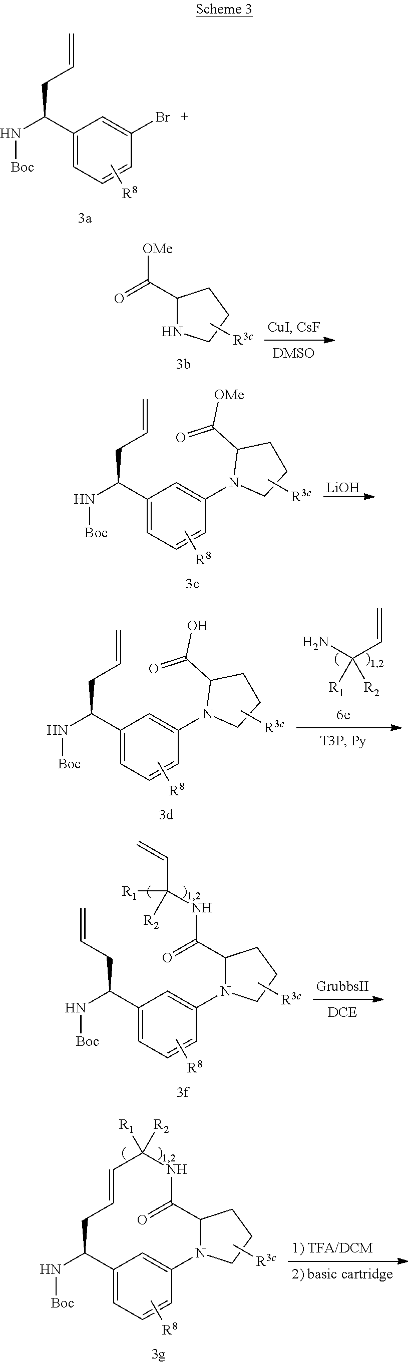

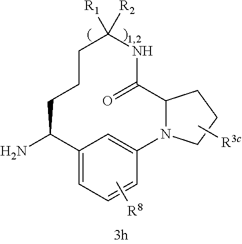

Representative compounds 4c of this invention can be prepared as described in Scheme 1. Using amide coupling conditions or condensation reactions of P precursors 1b with an appropriately substituted macrocycle amine 4a provides compounds 4c,

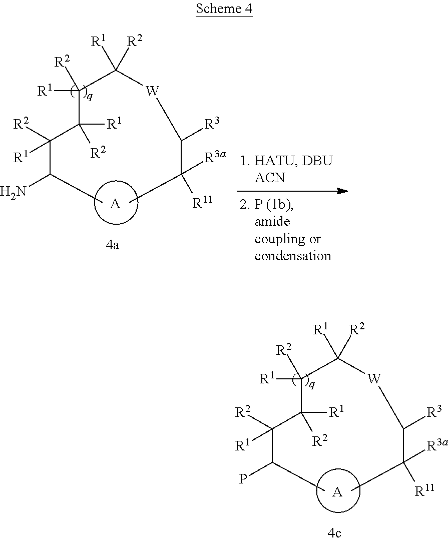

Representative macrocycle amine 5g of this invention can be made as shown in Scheme 4. Starting from 5a, coupling with 5b using Nigeishi reaction to give 5c. Coupling 5c with 5d using T3P to give 5e which goes through Grubbs II condition to form macrocycle 5f The double bond and protecting Cbz group can then be taken off under hydrogenation condition to provide microcycle amine 5g.

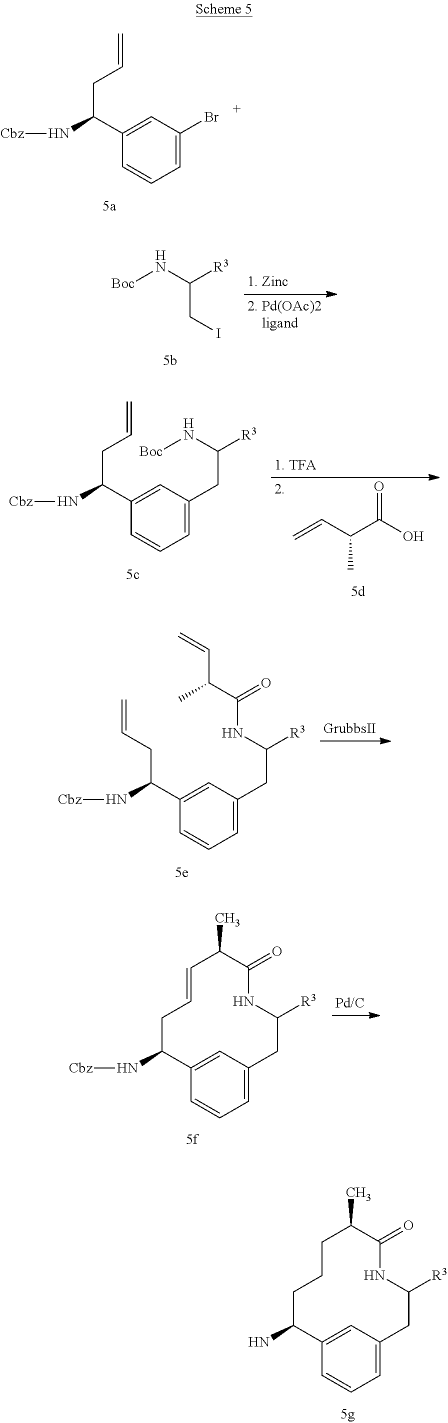

In some cases the R³ group on the macrocycle amine can be further functionalized. Examples are showed in Scheme 6 and 7.

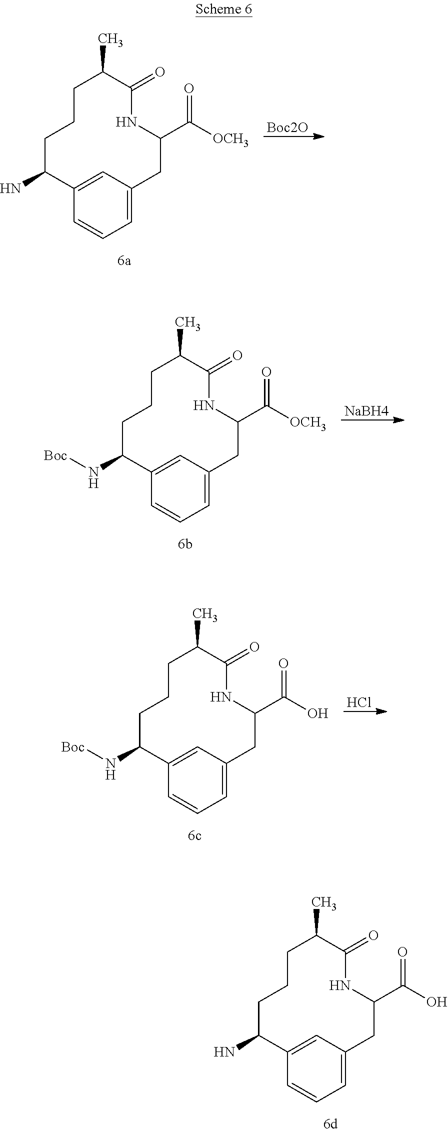

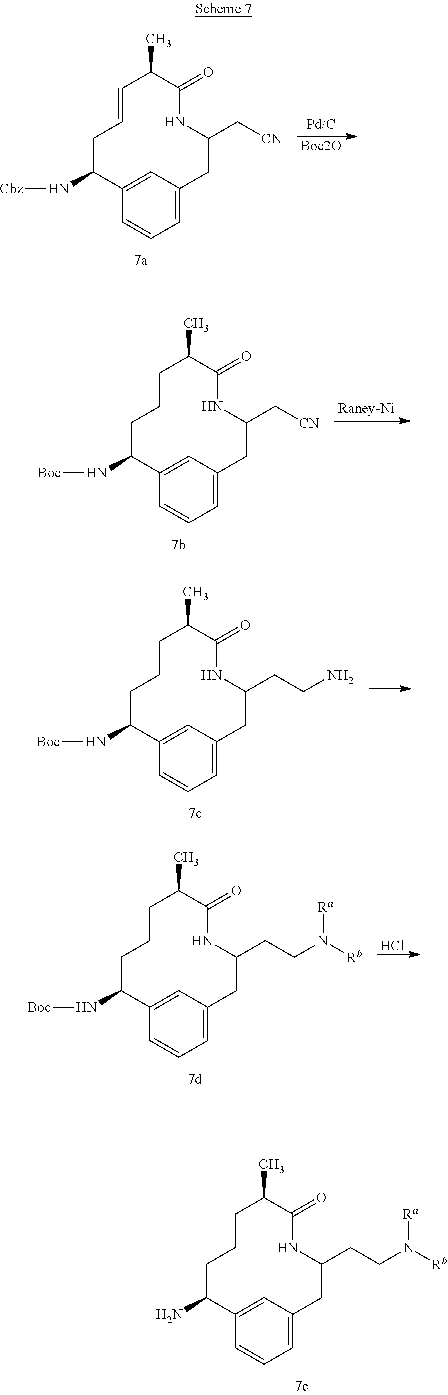

Compounds of the formula 6d and 7e can be converted to compounds in this invention according to Scheme 1.

Intermediates for preparation of compounds of the present invention corresponding to 8f can be prepared according to Scheme 8. Chloropyridine is reacted with the anion of an appropriate nitrile to yield 8b. The nitrile 8b is reduced with DIBAL and the resultant amine is acylated, to furnish diene 8c. Ring-closing metathesis is accomplished under the action of Grubbs' 2^{nd} generation catalyst to yield macrocycle 8d. Double bond of 8d can be reduced using either PtO2 or Pd/C to give 7e. The protecting Boc group can be removed under HCl or TFA.

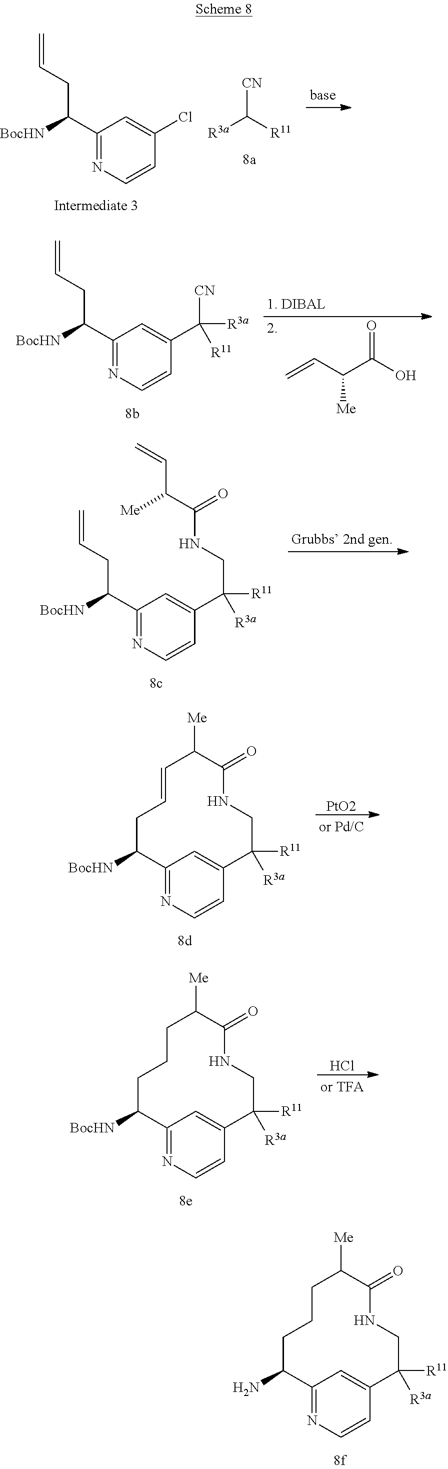

Compounds of the formula 8f can be converted to compounds in this invention according to Scheme 1.

Intermediates for preparation of compounds of the present invention corresponding to 9g can be prepared according to Scheme 9. Intermediate 1 is converted to the boronic acid 9a, which is subsequently subjected to a Suzuki reaction to afford 9b and the resultant amine is acylated with intermediate 15, to furnish diene 9c. Ring-closing metathesis is accomplished under the action of Grubbs' 2^{nd} generation catalyst to yield macrocycle 9d. Reduction of the olefin and pyridine ring is accomplished with PtO2 to give 9e. Acylation or reductive amination of 9e gives macrocycles with the general structure 9f. The protecting Boc group can be removed under TPA and free-basing affords 9g.

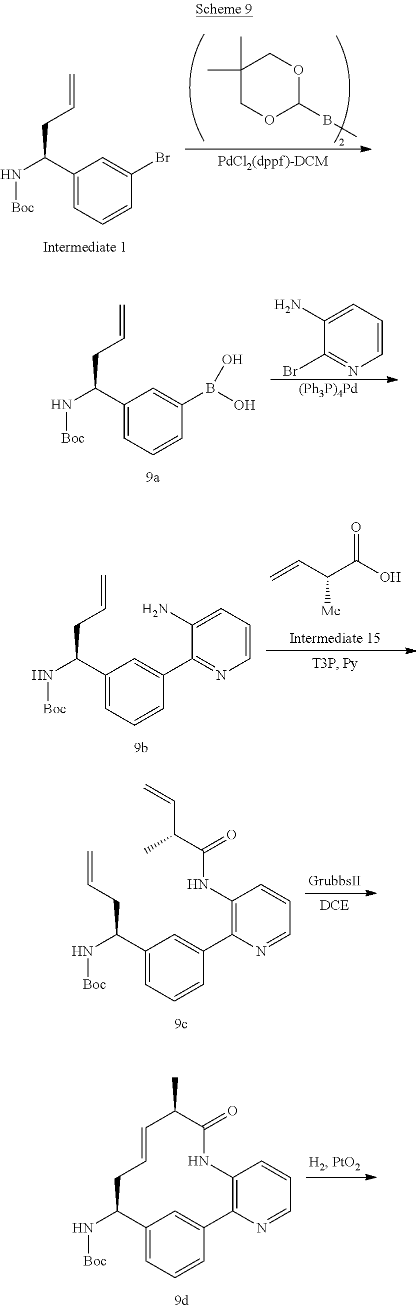

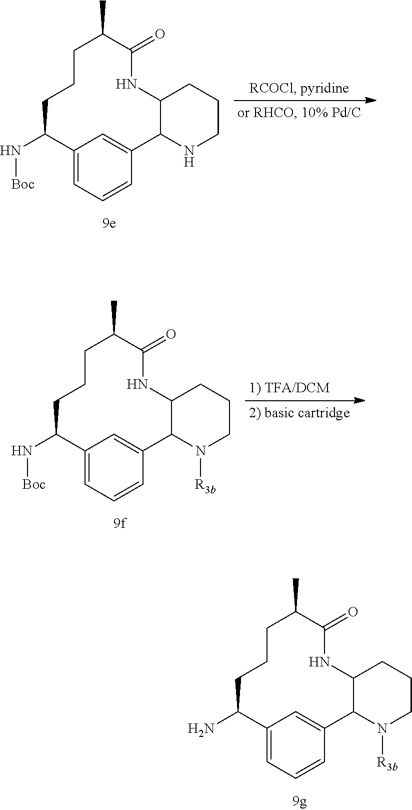

Compounds of the formula 9g can be converted to compounds in this invention according to Scheme 1.

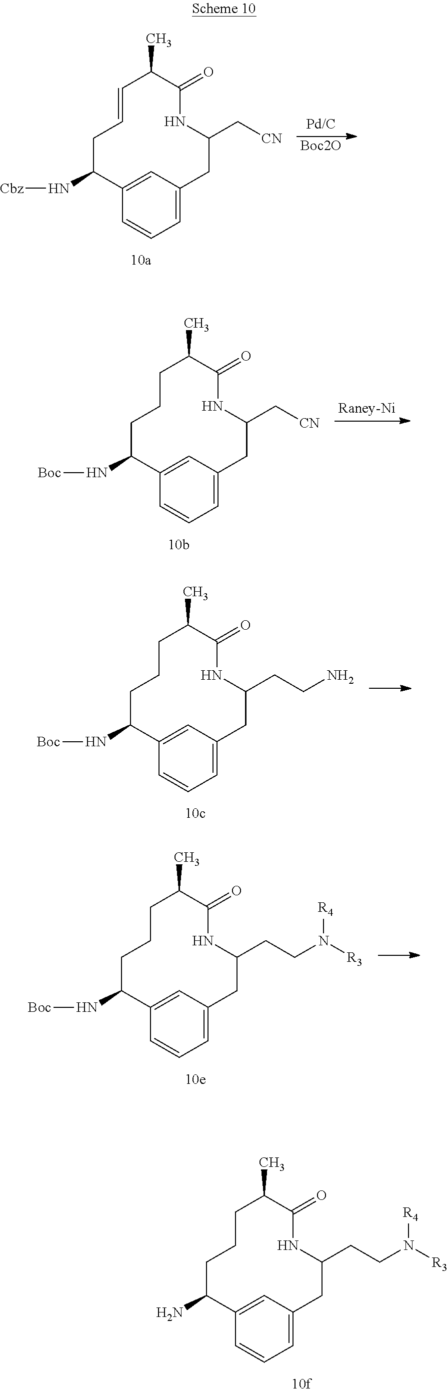

Compounds of the formula 10f can be converted to compounds in this invention according to Scheme 1.

Intermediates for preparation of compounds of the present invention wherein ring A is a six-membered heterocycle (example—pyridine) can be derived from appropriately substituted aldehydes 11a according to the general method outlined in Scheme 3. Condensation of aldehyde 11a (X═N) prepared according to a modified procedure described by Negi (Synthesis, 991 (1996)), with (S)-2-methylpropane-2-sulfinamide in the presence of anhydrous copper sulfate or cesium carbonate in a solvent such as DCM gives the sulfinimine 3b (Ellman, J., J. Org. Chem., 64:1278 (1999)). Using a modified procedure described by Kuduk (Tetrahedron Letters, 45:6641 (2004)), suitably substituted Grignard reagents, for example allylmagnesium bromide, can be added to sulfinimine 11b to give a sulfinamide 11c, as a mixture of diastereomers which can be separated at various stages of the sequence. The diastereoselectivity for the addition of allymagnesium bromide to sulfinimine 11b can be improved by employing indium(III) chloride according to a modified procedure of Xu (Xu, M-H, Organic Letters, 2008, 10 (6), 1259). Suzuki-Miyaura coupling between 4-chloropyridine 11c and an appropriately substituted aryl boronic acid or ester 11e in the presence of a base such as potassium phosphate, in a solvent mixture, such as DMSO and H₂O, or DMF, using a precatalyst such as Pd(dppf)Cl_{2.}CH₂Cl₂ complex provides 11g. Alternatively, the Suzuki-Miyaura coupling between boronic acid 11d and an appropriately substituted aryl halide 11f can be used to prepare 11g. Protecting group interconversion can be accomplished in two steps to give 11h. Alternatively, the protecting group interconversion can take place initially on 11e followed by the Suzuki-Miyaura coupling. The aniline 11h can then be coupled with an appropriately substituted carboxylic acid 3i using ®T3P and a base, such as pyridine, to give the amide 11j. Using a modified procedure described by Lovely (Tetrahedron Letters, 44:1379 (2003)), 11j, following pretreatment with p-toluenesulfonic acid to form the pyridinium ion, can be cyclized via ring-closing metathesis using a catalyst, such as Grubbs (II), in a suitable solvent, such as DCM, DCE, or toluene at elevated temperature, to give the pyridine-containing macrocycle 11k. The alkene can be reduced with hydrogen over either palladium on carbon or platinum oxide, and subsequent deprotection with TFA in DCM or 4M HCl in dioxane provides amine 11l. Compounds of the formula 11l can be converted to compounds in this invention according to Scheme 11.

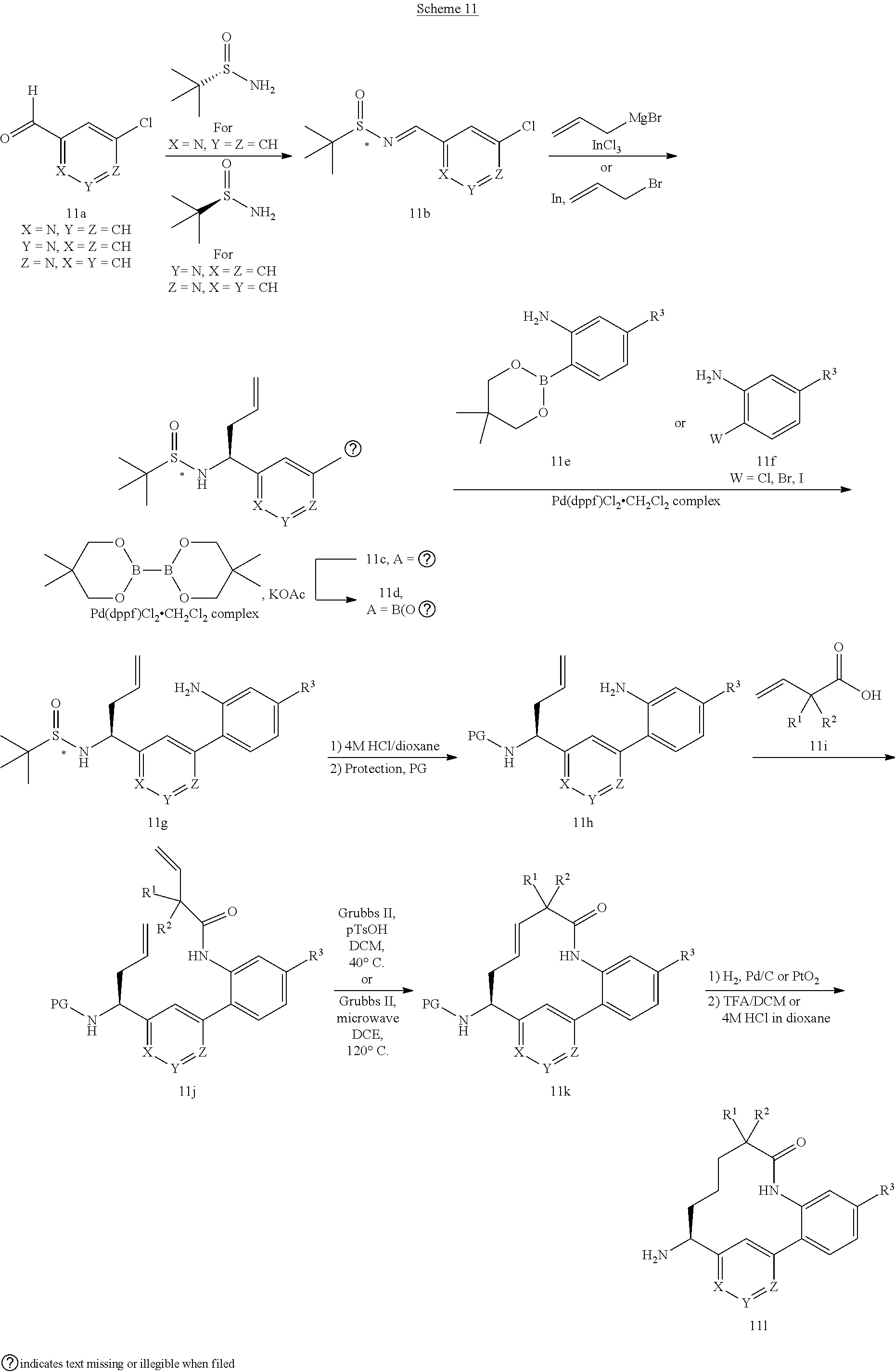

Compounds of the formula 11l can be converted to compounds in this invention according to Scheme 1.

Additional pyridine containing macrocycles useful for the synthesis of compounds of this invention can also be prepared according to Scheme 3. In cases where the pyridine core is a 4-pyridine (Z═N) rather than the 2-pyridine (X═N), conversion of 11h to 11j can be easily accomplished by using an acid chloride of 11i.

Methods for synthesis of a large variety of substituted pyridine compounds useful as starting materials for the preparation of compounds of the present invention are well known in the art and have been extensively reviewed. (For examples of methods useful for the preparation of pyridine starting materials see: Kroehnke, F., Synthesis, 1 (1976); Abramovitch, R. A., ed., “Pyridine and Its Derivatives”, The Chemistry of Heterocyclic Compounds, 14(Suppl, 1-4), John Wiley & Sons, New York (1974); Boulton, A. J. et al:, eds., Comprehensive Heterocyclic Chemistry, 2:165-524, Pergamon Press, New York (1984); McKillop. A., ed.. Comprehensive Heterocyclic Chemistry, 5:1-300, Pergamon Press, New York (1996)).

In cases where suitably substituted boronic acids are not commercially available, a modification to this approach may be adopted wherein an aryl halide is subjected to a palladium mediated coupling with a diboron species such as bis(pinacolato) diboron or bis(neopentyl glycolato)diboron to provide the corresponding 4,4,5,5-tetramethyl-[1,3,2]dioxaborolane or the 5,5-dimethyl-[1,3,2]dioxaborolane intermediates using the method of Ishiyama, T. et al. (J. Org. Chem., 60(23):7508-7510 (1995)). Alternately, this same intermediate can be prepared by reaction of the intermediate halide with the corresponding dialkoxyhydroborane as described by Murata et al. (J. Org. Chem., 62(19):6458-6459 (1997)). The boron pinacolate intermediates can be used in place of boronic acids for coupling to the aryl/heteroaryl halides or triflates or the boron pinacolate intermediate can be converted to the boronic acids. Alternately, the corresponding boronic acids can be prepared by metal-halogen exchange of the aryl/heteroaryl halide, quenching with a trialkoxyborate reagent, and aqueous workup to provide the boronic acids (Miyaura, N. et al., Chem. Rev., 95:2457 (1995)).

It is also realized that the scope of intermediate synthesis can be further extended outside the use of Suzuki-Miyaura coupling methodology since the precursor aryl halides or triflates described above are also precursors the Stille, Negishi, Hiyama, and Kumada-type cross coupling methodologies (Tsuji, J., Transition Metal Reagents and Catalysts: Innovations in Organic Synthesis, John Wiley & Sons (2000); Tsuji, J., Palladium Reagents and Catalysts: Innovations in Organic Synthesis, John Wiley & Sons (1996)).

Intermediates for preparation of compounds of the present invention wherein ring A is an imidazole ring, can be prepared from an appropriately N-protected allylpdycine (12a) according to the general method outlined in Scheme 4 (Contour-Galcera et al., Bioorg. Med. Chem. Lett., 11(5):741-745 (2001)). Condensation of 12a with a suitably substituted bromoacetophenone (12b) in the presence of a suitable base such as potassium bicarbonate, potassium carbonate or cesium carbonate in a suitable solvent such as DMF provides a keto ester intermediate which can be cyclized to afford an imidazole (12c) by heating in the presence of excess ammonium acetate in a solvent such as toluene or xylene. This latter transformation can be conveniently carried out on small scale at 160° C. in a microwave reactor or on larger scale by refluxing the mixture while removing water via a Dean-Stark trap. The resulting imidazole intermediate (12c) is then protected by treatment with SEM-Cl in the presence of a base such as sodium hydride or dicyclohexylmethylamine in a solvent such as THF or DCM. The resulting aryl bromide (12d) is then converted to the corresponding aniline (12e) by heating in a sealed vessel with excess ammonium hydroxide, in the presence of copper iodide, a base such as potassium carbonate and a catalytic amount of proline in DMSO as solvent. Acylation of 12e with the appropriate alkenoic acid and a coupling agent such as T3P® or BOP reagent, or alternately, by treatment with an alkenoic acid chloride in the presence of a base such as TEA, DIPEA, or pyridine provides diene 12f, which undergoes ring closing metathesis by heating in dilute solution in the presence of p-toluene sulfonic acid and 2^{nd} Generation Grubbs Catalyst in a suitable solvent such as DCM or DCE to provide the corresponding macrocycle (12g). Alternately, the RCM can be run in a microwave at elevated temperatures without pTsOH. Reduction of the double bond followed by bromination with NBS at room temperature affords 12k. Suzuki-Miyaura coupling with methylboronic acid or tetramethylstannane and removal of the protecting group (PG), provides intermediate 12i. Intermediates 12i can be converted to compounds of the present invention following the steps described in Scheme 1.

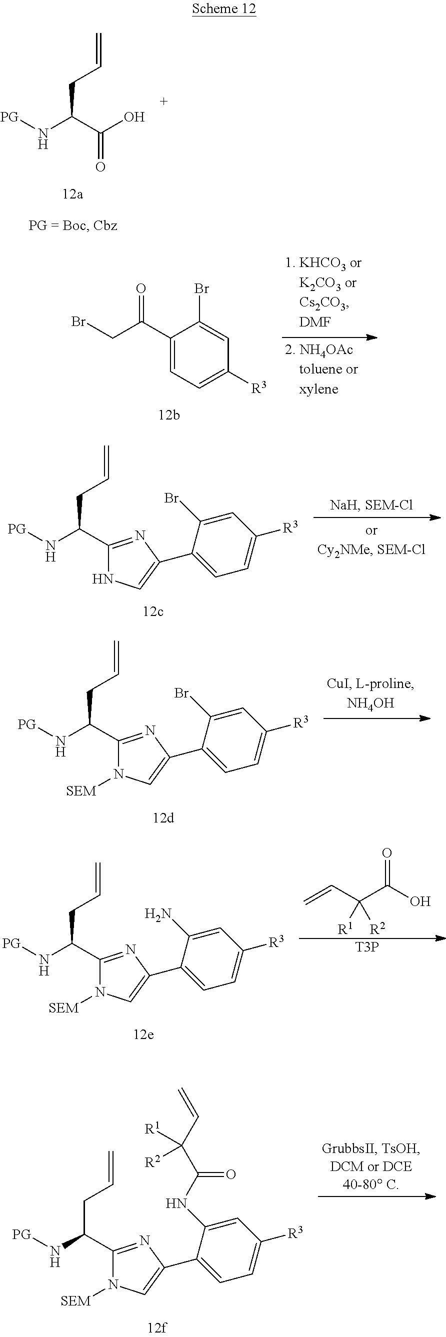

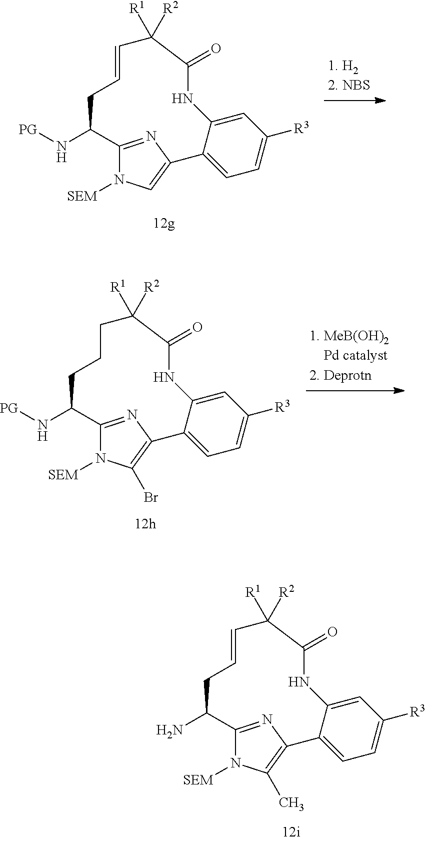

What is claimed is:

1. A compound of Formula (I);
P-Ma (I)
or a stereoisomer, an enantiomer, a diastereomer, a tautomer, a pharmaceutically acceptable salt thereof, wherein:
Ma is independently selected from Formulae (M1), (M2), (M3), and (M4): 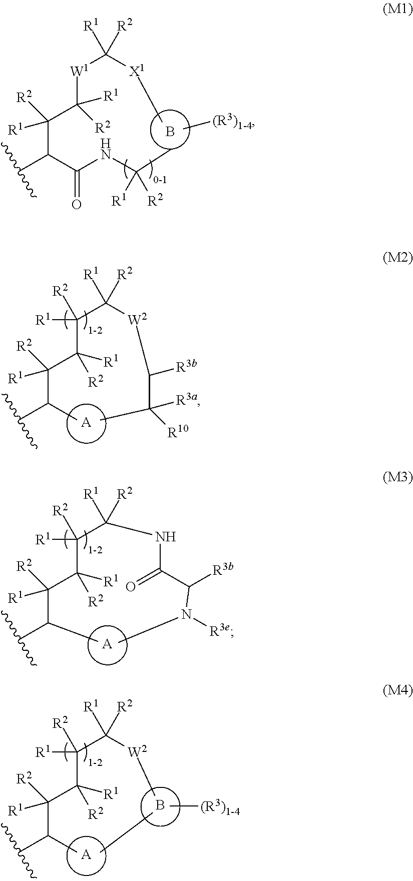
P is independently selected from Formulae (P1), (P2), (P3), (P4), (P5), (P6), (P7), (P8), P(9), P(10), P(11), P(12), P(13), and P(14): 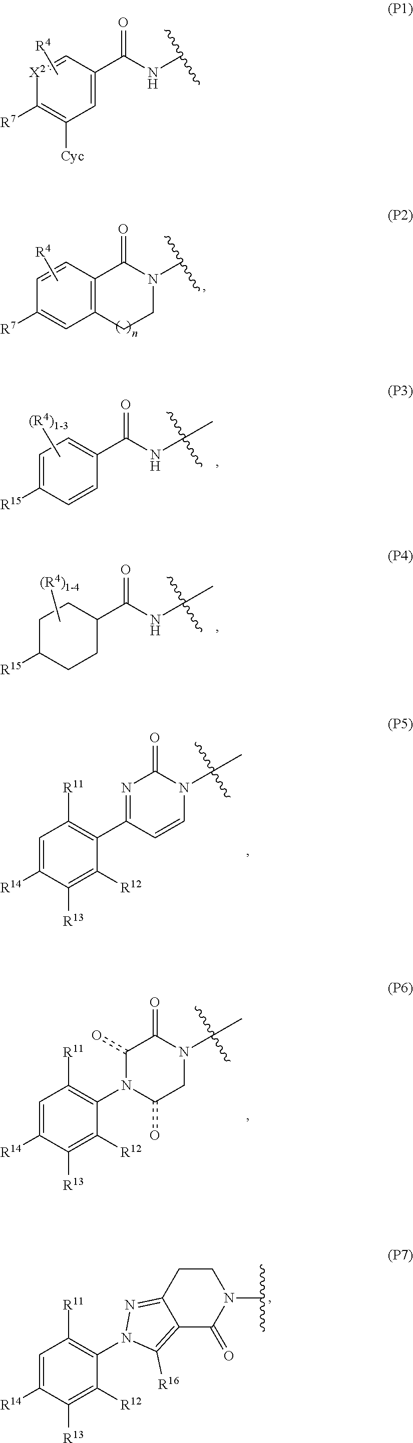 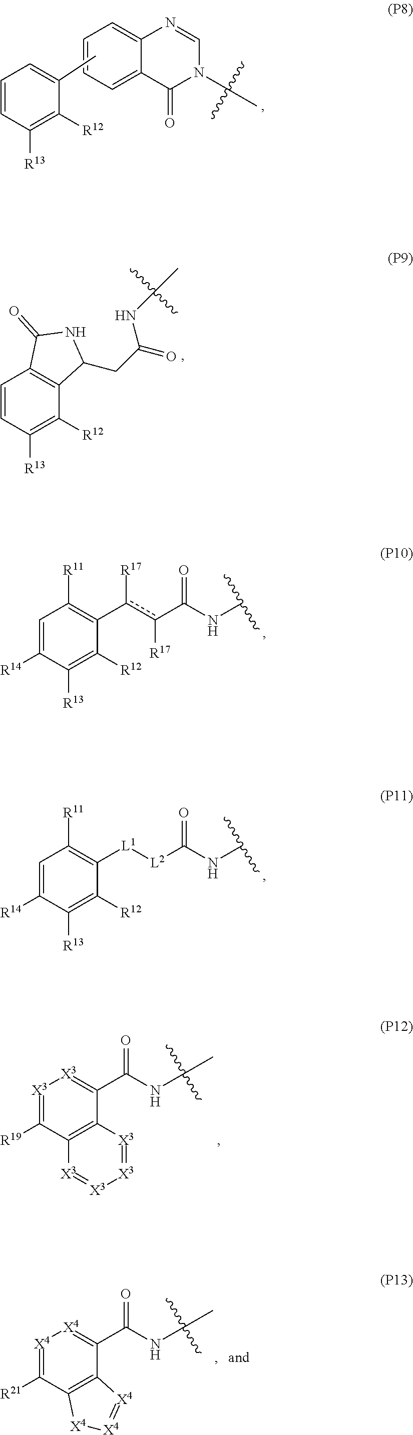 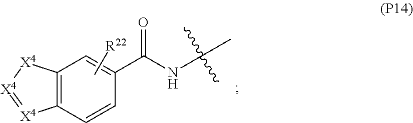
Cyc is C₃₋₆ cycloalkyl;
ring A is independently selected from 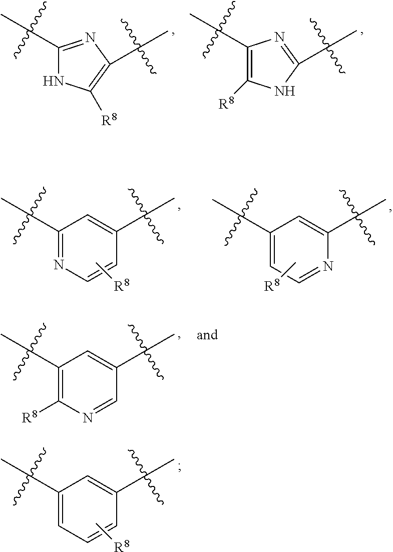
Ring B is independently selected from aryl and 5- to 10-membered heterocyclyl;
W¹ is independently selected from (CR¹R²)₁₋₂, O, NH, and N(C₁₋₄ alkyl);
W² is independently selected from —C(O)NH, —NHC(O)—, and —NHC(O)CH₂—;
X¹ is independently selected from —CR²³NH—, —NHC(═O)— and —C(═O)NH—; provided when W¹ is O, NH, and N(C₁₋₄ alkyl). X¹ is not —NHC(═O)—;
X² is independently selected from N and CR⁴;
X³ is independently selected from CR¹⁸ and N;
X⁴ is independently selected from CR²⁰, O, S, N, NH, and NC₁₋₄alkyl;
L¹ is independently selected from CH₂, O, NH, NC₁₋₄alkyl, and S;
L² is independently selected from NH and NC₁₋₄alkyl;
R¹ and R² are independently selected from H, halogen. C₁₋₄alkyl substituted with 0-4 R^{e}, OR^{b}, and C₃₋₅ cycloalkyl substituted with 1-4 R⁶;
alternatively, two adjacent R¹ groups form a bond;
R³ is independently selected from H, NO₂, ═O, halogen, C₁₋₄ alkyl substituted with 1-5 R⁵, C₂₋₄ alkenyl substituted with 1-5 R⁵, C₂₋₄ alkynyl substituted with 1-5 R⁵, CN, —(CH₂)ₙ—OR^{b}, —(CH₂)ₙ—NR^{a}R^{a}, —(CH₂)ₙ—C(═O)R^{b}, —(CH₂)ₙ—C(═O)OR^{b}, —(CH₂)ₙ—NR^{a}C(═O)OR^{b}, —(CH₂)ₙ—NR^{a}C(═O)R^{b}, —(CH₂)ₙ—NR^{a}C(N—CN)NR^{a}R^{a}, —(CH₂)ₙ—NR^{a}C(NH)NR^{a}R^{a}, —(CH₂)ₙ—N═CR^{b}NR^{a}R^{b}, —(CH₂)ₙ—NR^{a}C(═O)NR^{a}R^{a}, —(CH₂)ₙ—C(═O)NR^{a}R^{a}, —(CH₂)ₙ—NR^{a}C(═S)NR^{a}C(═O)R^{b}, —(CH₂)ₙ—S(═O)ₚR^{c}, —(CH₂)ₙ—S(═O)ₚNR^{a}R^{a}, —(CH₂)ₙ—NR^{a}S(═O)ₚNR^{a}R^{a}, —(CH₂)ₙ—NR^{a}S(═O)ₚR^{c}, —(CR_{d}R_{d})ₙ—C₃₋₁₀ carbocyclyl substituted with 1-5 R⁵, and —(CR_{d}R_{d})ₙ-4- to 10-membered heterocyclyl substituted with 1-5 R⁵; optionally, two adjacent R³ groups on the carbocyclyl and heterocyclyl may form a ring substituted with 1-5 R⁵;
R^{3a} is independently selected from H and C₁₋₄alkyl;
R^{3b} is independently selected from H, substituted with 1-5 R⁵, C₂₋₄ alkenyl substituted with 1-5 R⁵, C₂₋₄ alkynyl substituted with 1-5 R⁵, —(CH₂)ₙ—CN, —(CH₂)ₙ—OR^{b}, —(CH₂)ₙ—OC(═O)NR^{a}R^{a}, —(CH₂)ₙ—NR^{a}R^{a}, —(CH₂)ₙ—C(═O)R^{b}, —(CH₂)ₙ—C(═O)OR^{b}, —(CH₂)ₙ—NR^{a}C(═O)OR^{b}, —(CH₂)ₙ—NR^{a}C(═O)R^{b}, —(CH₂)ₙ—NR^{a}C(N—CN)NR^{a}R^{a}, —(CH₂)ₙ—NR^{a}C(NH)NR^{a}R^{a}, —(CH₂)ₙ—N═CR^{b}NR^{a}R^{a}, —(CH₂)ₙ—NR^{a}C(═O)NR^{a}R^{a}, —(CH₂)ₙ—C(═O)NR^{a}R^{a}, —(CH₂)ₙ—NR^{a}C(═S)NR^{a}C(═O)R^{b}, —(CH₂)ₙ—S(═O)ₚR^{c}, —(CH₂)ₙ—S(═O)ₚNR^{a}R^{a}, —(CH₂)ₙ—NR^{a}S(═O)ₚNR^{a}R^{a}, —(CH₂)ₙ—NR^{a}S(═O)ₚR^{c}, —(CR_{d}R_{d})ₙ—C₃₋₁₀ carbocyclyl substituted with 1-5 R⁵, and —(CR_{d}R_{d})ₙ-4- to 10-membered heterocyclyl substituted with 1-5 R⁵; optionally, two adjacent R³ groups on the carbocyclyl and heterocyclyl may form a ring substituted with 1-5 R⁵;
alternatively, R^{3a} and R^{3b} are taken together to form a C₃₋₆ carbocyclic or heterocyclic ring comprising carbon atoms and 1-3 heteroatoms selected from O, NR^{3c}, S, wherein the carbocyclic or heterocyclic ring is substituted with R^{3d};
alternatively, R^{3a} and R¹⁰ are taken together to form a C₃₋₆ carbocyclic or heterocyclic ring comprising carbon atoms and 1-3 hetero atoms selected from N, NR^{3e}, O, and S, wherein the carbocyclic or heterocyclic ring is substituted with R^{3d};
R^{3e} is independently selected from H, C₁₋₄alkyl substituted with 1-5 R⁵, —(CH₂)ₙ—C(═O)R^{b}, —(CH₂)ₙ—C(═O)OR^{b}, —(CH₂)ₙ—C(═O)NR^{a}R^{a}, —(CH₂)ₙ—NHC(═O)OR^{b}, —(CH₂)ₙ—S(═O)ₚR^{c}, —(CH₂)ₙ—S(═O)ₚNR^{a}R^{a}, —(CR_{d}R_{d})ₙ—C₃₋₁₀ carbocyclyl substituted with 1-5 R⁵, and —(CR_{d}R_{d})ₙ-4- to 10-membered heterocyclyl substituted with 1-5 R⁵;
R^{3d} is independently selected from H, NO₂, ═O, halogen, C₁₋₄ alkyl substituted with 1-5 R⁵, C₂₋₄ alkenyl substituted with 1-5 R⁵, C₂₋₄ alkynyl substituted with 1-5 R⁵, CN, —(CH₂)ₙ—OR^{b}, —(CH₂)ₙ—NR^{a}R^{a}, —(CH₂)ₙ—C(═O)R^{b}, —(CH₂)ₙ—C(═O)OR^{b}, —(CH₂)ₙ—NR^{a}C(═O)OR^{b}, —(CH₂)ₙ—NR^{a}C(═O)R^{b}, —(CH₂)ₙ—NR^{a}C(N—CN)NR^{a}R^{a}, —(CH₂)ₙ—NR^{a}C(NH)NR^{a}R^{a}, —(CH₂)ₙ—N═CR^{b}NR^{a}R^{a}, —(CH₂)ₙ—NR^{a}C(═O)NR^{a}R^{a}, —(CH₂)ₙ—C(═O)NR^{a}R^{a}, —(CH₂)ₙ—NR^{a}C(═S)NR^{a}C(═O)R^{b}, —(CH₂)ₙ—S(═O)ₚR^{c}, —(CH₂)ₙ—S(═O)ₚNR^{a}R^{a}, —(CH₂)ₙ—NR^{a}S(═O)ₚNR^{a}R^{a}, —(CH₂)ₙ—NR^{a}S(═O)ₚR^{c}, —(CR_{d}R_{d})ₙ—C₃₋₁₀ carbocyclyl substituted with 1-5 R⁵, and —(CR_{d}R_{d})ₙ-4- to 10-membered heterocyclyl substituted with 1-5 R⁵;
R^{3e} is independently selected from H and C₁₋₄alkyl;
alternatively, R^{3b} and R^{3e} are taken together to form a heterocyclic ring compring carbon atoms and 1-3 heteroatoms selected from O, NR^{3c}, S, wherein the heterocyclic ring is substituted with R^{3d};
R⁴ is independently selected from H, halogen, CN, C₁₋₆ alkyl substituted with 1-5 R⁹, and C₃₋₆ cycloalkyl substituted with 1-5 R⁹;
R⁵, at each occurrence, is independently selected from H, D, —(CH₂)ₙ—OR^{b}, ═O, —(CH₂)ₙNH₂, —(CH₂)ₙCN, halogen, C₁₋₆ alkyl, —(CH₂)ₙ—C(═O)OR^{b}, —(CH₂)ₙ—OR^{b}, —(CH₂)ₙ—C₃₋₁₀ carbocyclyl substituted with 0-5 R^{e}, —(CH₂)ₙ-4- to 10-membered heterocyclyl substituted with 0-5 R^{e}, and —O-4- to 10-membered heterocyclyl substituted with 0-5 R^{e};
R⁶ is independently selected from H, OH, ═H, —(CH₂)ₙNH₂, —(CH₂)ₙCN, halogen, C₁₋₆ alkyl, —(CH₂)ₙ—C(═O)OH, —(CH₂)ₙ—C(═O)OC₁₋₄alkyl, —(CH₂)ₙ—OC₁₋₄alkyl, —(CH₂)ₙ—C₃₋₁₀ carbocycle substituted with 0-5 R^{e}, —(CH₃)ₙ-4- to 10-membered heterocycle substituted with 0-5 R^{e}, and —(CH₂)ₙ-4- to 10-membered heterocycle substituted with 0-5 R^{e};
R⁷ is —(CH₂)ₙNH₂;
R⁸ is independently selected from H, OH, F, Cl, Br, C₁₋₄alkyl, C₁₋₄alkoxy, CF₃, CN, C₃₋₆ cycloalkyl, aryl, and 5- to 6-membered heterocycle;
R⁹, at each occurrence, is independently selected from H, halogen and OH;
R¹⁰ is independently selected from H, C₁₋₄alkyl, and aryl;
R¹¹ is independently selected from H, halogen, CN, OCH₃, OCF₃, CH₃, C(═O)CH₃, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴;
R¹² is independently selected from H and halogen;
R¹³ is independently selected from H, F, Cl, methyl, ethyl, isopropyl, and OCH₃;
R¹⁴ is independently selected from H, F, and Cl;
R¹⁵ is independently selected from pyrazolyl, aminopyrazolyl, pyrrolidinyl, triazolonyl, aminothiadiazolyl, oxazolyl, thiazolyl, pyridinyl, pyrimidinyl, and 2-pyridonyl;
R¹⁶ is independently selected from H, CN, OR^{b}, halogen, NR^{a}R^{a}, and C₁₋₃ alkyl substituted with 0-5 R^{e};
R¹⁷ is independently selected from H, methyl and halogen;
R¹⁸ is independently selected from H, methyl and halogen;
R¹⁹ is independently selected from C₁₋₄ alkyl, OC₁₋₄alkyl, SC₁₋₄alkyl, and C₃₋₆ cycloalkyl;
R²⁰ is independently selected from H, halogen, CN, and C₁₋₄alkyl;
R²¹ is independently selected from C₁₋₄alkyl, OC₁₋₄ alkyl, SC₁₋₄ alkyl, and C₃₋₆ cycloalkyl;
R²² is independently selected from C₁₋₄ alkyl and NR^{a}R^{a};
R²³ is independently selected from H, C₁₋₄alkyl substituted with 0-3 R^{e}, CO₂H, —(CH₂)ₙCO₂(C₁₋₄ alkyl), —(CH₂)ₙCO₂NH₂, —CONH(C₁₋₄ alkoxy), —CO₂(CH₂)₂O(C₁₋₄ alkyl), —CO₂(CH₂)₂N(C₁₋₄alkyl)₂, —CONH(CH₂)₂O(C₁₋₄ alkyl), —CONH(CH₂)₂N(C₁₋₄ alkyl)₂, —CON(C₁₋₄alkyl)(CH₂)₂O(C₁₋₄alkyl), —CON(C₁₋₄alkyl)(CH₂)₂N(C₁₋₄alkyl)₂, —CONHBn, —CONH(OBn), and —(CO)₀₋₁(CH₂)₀₋₃—C₃₋₆ carbocycle;
R²⁴, at each occurrence, is independently selected from H, halogen, CN, NO₂, ═O, C(═O)NR^{a}R^{a}, C(═O)OR^{b}, Si(C₁₋₄ alkyl)₃, —(CH₂)ₙ—OR^{b}, —(CH₂)ₙ—NR^{a}R^{a}, C(═NOH)NH₂, C₁₋₆ alkyl substituted with 0-5 R^{e}, C₂₋₆ alkenyl substituted with 0-5 R^{e}, C₂₋₆ alkynyl substituted with 0-5 R^{e}, aryl substituted with 0-5 R^{e}, —(CH₂)ₙ—C₃₋₆cycloalkyl substituted with 0-5 R^{e}, —(CH₂)ₙ—O-4- to 10-membered heterocyclyl substituted with 0-5 R^{e};
R^{a}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};
R^{b}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e};
R^{c}, at each occurrence, is independently selected from C₁₋₆ alkyl substituted with 0-5 R^{e}, C₂₋₆alkenyl substituted with 0-5 R^{e}, C₂₋₆alkynyl substituted with 0-5 R^{e}, C₃₋₆carbocyclyl, and heterocyclyl;
R^{d}, at each occurrence, is independently selected from H and C₁₋₄alkyl substituted with 0-5 R^{e};
R^{e}, at each occurrence, is independently selected from F, Cl, Br, CN, NO₂, ═O, C₁₋₆ alkyl substituted with 0-5 R^{f}, C₂₋₆ alkenyl, C₂₋₆ alkynyl, —(CH₂)ₙ—C₃₋₆ cycloalkyl, —(CH₂)ₙ-aryl, —(CH₂)ₙ-heterocyclyl, CO₂H, —(CH₂)ₙOR^{f}, SR^{f}, and —(CH₂)₁NR^{f}R^{f};
R^{f}, at each occurrence, is independently selected from H, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl;
n, at each occurrence, is an integer independently selected from 0, 1, 2, and 4; and
p, at each occurrence, is an integer independently selected from 0, 1, and 2.

2. The compound according to claim 1, or a stereoisomer, an enantiomer, a diastereomer, a tautomer, a pharmaceutically acceptable salt thereof, wherein:
Ma is (M1-I): 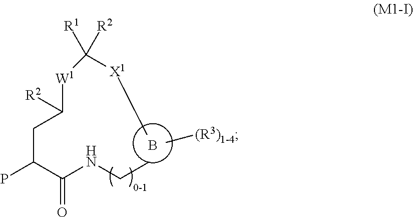
P is independently selected from Formulae (P1), (P2), (P3), (P5), (P6), (P7), (P8), P(9), P(10), P(11), P(12), P(13), and P(14): 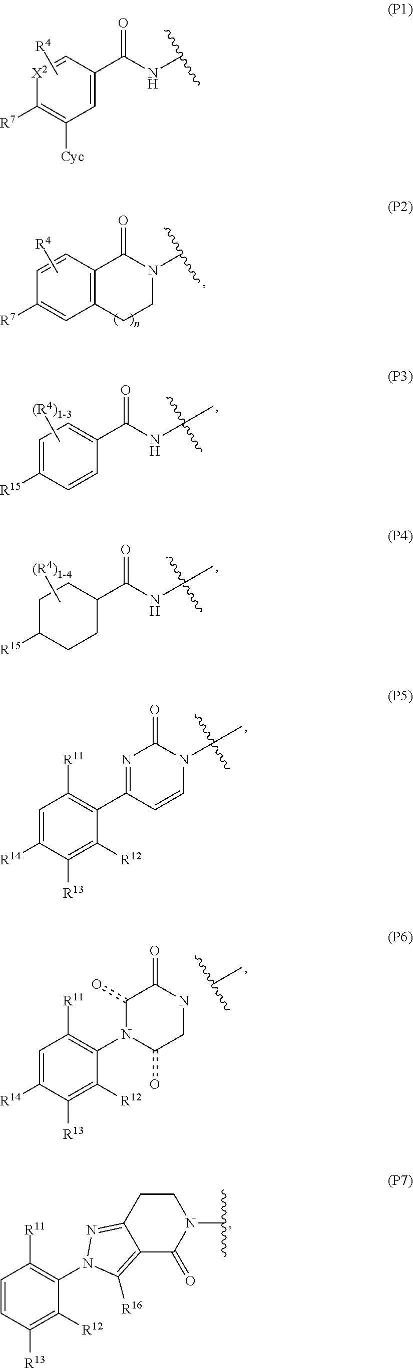 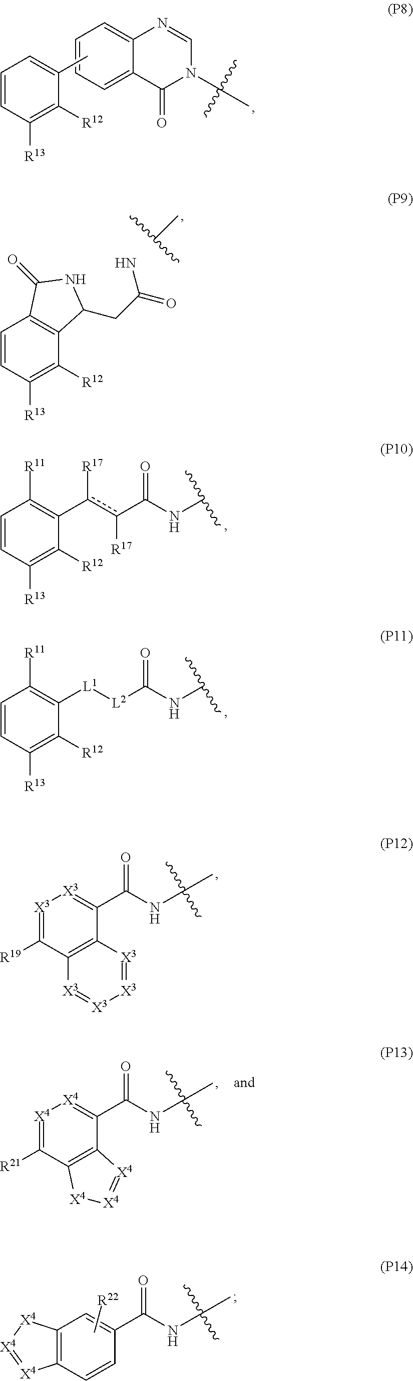
Cyc is C₃₋₆ cycloalkyl;
Ring B is independently selected from aryl and 5- to 6-membered heterocyclyl;
W¹ is independently selected from (CHR^{1a})₁₋₂, O, NH, and N(C₁₋₄ alkyl);
X¹ is independently selected from —NHC(═O)— and —C(═O)NH—; provided when W¹ is O, NH, and N(C₁₋₄ alkyl), X¹ is not —NHC(═O)—;
X² is independently selected from N and CR⁴;
X³ is independently selected from CR¹⁸ and N;
X⁴ is independently selected from CR²⁰, O, S, N, NH, and NC₁₋₄alkyl;
L¹ is independently selected from CH₂, O, NH, NC₁₋₄alkyl, and S;
L² is independently selected from NH and NC₁₋₄alkyl;
R¹ is independently selected from H and C₁₋₄alkyl;
R^{1a} is independently selected from H, F, CH₃, and OH;
R² is independently selected from H and OH;
R³ is independently selected from H, ═O, halogen, C₁₋₄alkyl substituted with 1-5 R⁵, CN, —NR^{a}R^{a}, —(CH₂)ₙ—C(═O)R^{b}, —(CH₂)ₙ—C(═O)OR^{b}, —NR^{a}C(═O)OR^{b}, —NR^{a}C(═O)R^{b}, —NR^{a}C(═O)NR^{a}R^{a}, —C(═O)NR^{a}R^{a}, —S(═O)ₚR^{e}, —(CH₂)ₙ—C₃₋₁₀ carbocyclyl substituted with 1-5 R⁵, and —(CH₂)ₙ-4-to 10-membered heterocyclyl substituted with 1-5 R⁵; optionally, two adjacent R³ groups on the carbocyclyl and heterocyclyl may form a ring substituted with 1-5 R⁵:
R⁴ is independently selected from H, halogen, CN, C₁₋₆ alkyl substituted with 1-5 R⁹, and C₃₋₆ cycloalkyl substituted with 1-5 R⁹;
R⁵, at each occurrence, is independently selected from H, D, —(CH₂)ₙ—OR^{b}, ═O, —(CH₂)ₙNH₂, —(CH₂)ₙCN, halogen, C₁₋₆alkyl, —(CH₂)ₙ—C(═O)OR^{b}, —(CH₂)ₙ-OR^{b}, —(CH₂)ₙ—C₃₋₁₀ carbocyclyl substituted with 0-5 R^{e}, —(CH₂)ₙ-4- to 10-membered heterocyclyl substituted with 0-5 R^{e}, and —O-4- to 10-membered heterocyclyl substituted with 0-5 R^{e};
R⁷ is —(CH₂)ₙNH₂,
R⁹, at each occurrence, is independently selected from H, halogen and OH;
R¹¹ is independently selected from H, halogen, CN, OCH₃, OCF₃, CH₃, C(═O)CH₃, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴;
R¹² is independently selected from H and halogen;
R¹³ is independently selected from H, F, Cl, methyl, ethyl, isopropyl, and OCH_{3;}
R¹⁴ is independently selected from H, F, and Cl;
R¹⁵ is independently selected from pyrazolyl, aminopyrazolyl, pyrrolidinyl, triazolonyl, aminothiadiazolyl, oxazolyl, thiazolyl, pyridinyl, pyrimidinyl, and 2-pyridonyl;
R¹⁶ is independently selected from H, CN, OR^{b}, halogen, NR^{a}R^{a}, and C₁₋₃ alkyl substituted with 0-5 R^{e};
R¹⁷ is independently selected from H, methyl and halogen;
R.¹⁸ is independently selected from H, methyl and halogen;
R¹⁹ is independently selected from C₁₋₄ alkyl, OC₁₋₄ alkyl, SC₁₋₄ alkyl, and C₃₋₆ cycloalkyl;
R²⁰ is independently selected from H, halogen, CN, and C₁₋₄alkyl;
R²¹ is independently selected from C₁₋₄alkyl, OC₁₋₄ alkyl, SC₁₋₄ alkyl, and C₃₋₆ cycloalkyl;
R²² is independently selected from C₁₋₄alkyl and NR^{a}R^{a};
R²⁴, at each occurrence, is independently selected from H, halogen, CN, NO₂, ═O, C(═O)NR^{a}R^{a}, C(═O)OR^{b}, —(CH₂)ₙ—OR^{b}, —(CH₂)ₙ—NR^{a}R^{a}, C(═NOH)NH₂, C₁₋₄ alkyl substituted with 0-5 R^{e}, C₂₋₆ alkenyl substituted with 0-5 R^{e}, C₂₋₆ alkynyl substituted with 0-5 R^{e}, aryl substituted with 0-5 R^{e}, —(CH₂)ₙ—C₃₋₆ cycloalkyl substituted with 0-5 R^{e}, —(CH₂)ₙ—O-4- to 10-membered heterocyclyl substituted with 0-5 R^{e};
R^{a}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ-C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};
R^{b}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R_{c}, —(CH₂)ₙ—C₃₋₁₀carbocyclyl substituted with 0-5 R^{e}, and —(CH₂)ₙ-heterocyclyl substituted with 0-5 R^{e};
R^{e}, at each occurrence, is independently selected from C₁₋₆ alkyl substituted with 0-5 R^{e}, C₂₋₆alkenyl substituted with 0-5 R″, C₂₋₆alkynyl substituted with 0-5 R^{e}, C₃₋₆ carbocyclyl, and heterocyclyl;
R^{d}, at each occurrence, is independently selected from H and C₁₋₄alkyl substituted with 0-5 R^{e};
R^{e}, at each occurrence, is independently selected from F, Cl, Br, CN, NO₂, ═O, C₁₋₆ alkyl substituted with 0-5 R^{f}, C₂₋₆ alkenyl, C₂₋₆ alkynyl, —(CH₂)ₙ—C₃₋₆ cycloalkyl, —(CH₂)ₙ-aryl, —(CH₂)ₙ-heterocyclyl, CO₂H, —(CH₂)ₙOR^{f}, SR^{f}, and —(CH₂)ₙNR^{f}R^{f};
R^{f}, at each occurrence, is independently selected from H, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl;
n, at each occurrence, is an integer independently selected from 0, 1. 2, and 4; and
p, at each occurrence, is an integer independently selected from 0, 1, and 2.

3. The compound according to claim 2, or a stereoisomer, a stereoisomer, enantiomer, a tautomer, a pharmaceutically acceptable salt thereof wherein:
ring B is independently selected from 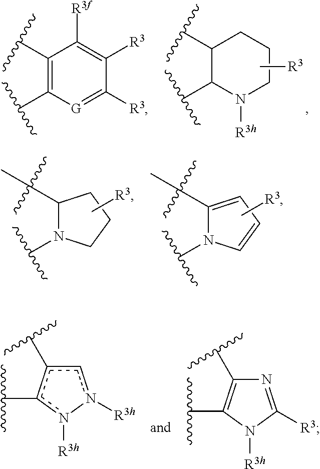
G is independently selected from N and CR^{3g};
R³ is independently selected from H, ═O, halogen, C₁₋₄ alkyl substituted with 1-5 R⁵, CN, —NR^{a}R^{a}, —C(═O)R^{b}, —C(═O)OR^{b}, —NR^{a}C(═O)OR^{b}, —NR^{a}C(═O)R^{b}, —NR^{a}C(═O)NR^{a}R^{a}, —C(═O)NR^{a}R^{a}, —S(═O)ₚR^{c}, —(CH₂)ₙ—C₃₋₁₀ carbocyclyl substituted with 1-5 R⁵, and —(CH₂)ₙ-4- to 10-membered heterocyclyl substituted with 1-5 R⁵;
R^{3f} is independently selected from H and halogen;
R^{3g} is independently selected from H, halogen, methyl, and CN;
R^{3h} is absent or independently selected from H, C₁₋₄alkyl substituted with 1-4 R⁵, —(CH₂)ₙ—C(O)R^{b}, —(CH₂)ₙ—C(═O)OR^{b}; and
R⁵, at each occurrence, is independently selected from H, D, —OR^{b}, —NH₂, —CN, halogen, C₁₋₆ alkyl, —C(═O)OR^{b}, C₃₋₁₀ carbocyclyl substituted with 0-5 R^{e}, -4- to 10-membered heterocyclyl substituted with 0-5 R^{e}, and —O-4- to 10-membered heterocyclyl substituted with 0-5 R^{e}.

4. The compound according to claim 1, or a stereoisomer, an enantiomer, a diastereomer, a tautomer, a pharmaceutically acceptable salt thereof, wherein:
Ma is (M2-1): 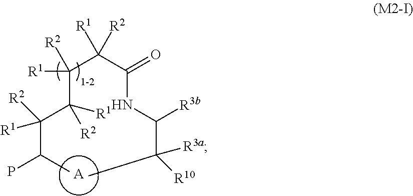
P is independently selected from Formulae (P1), (P2), (P3), (P4), (P5), (P6), (P7), (P8), P(9), P(10), P(11), P(12), P(13), and. P(14): 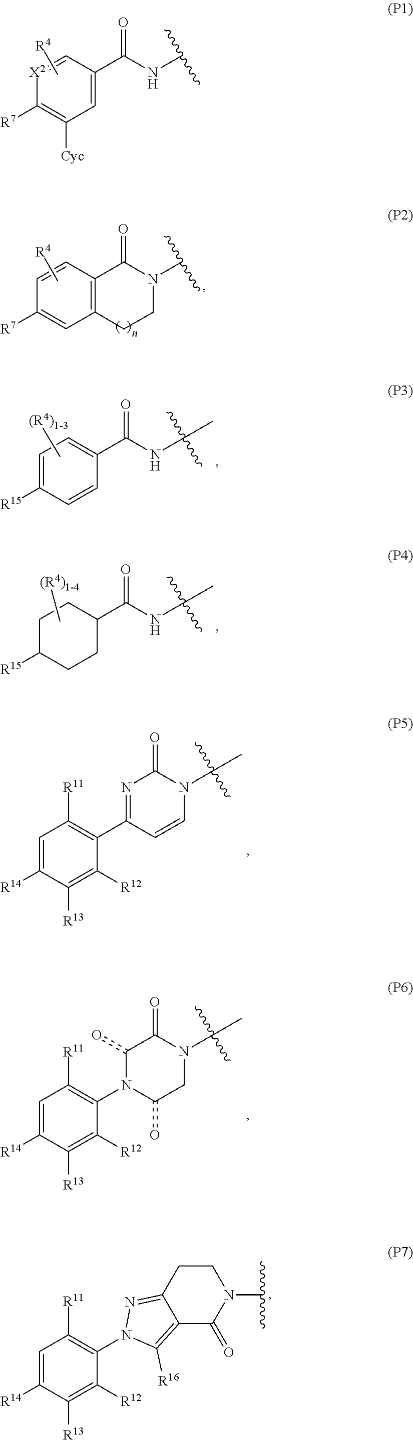 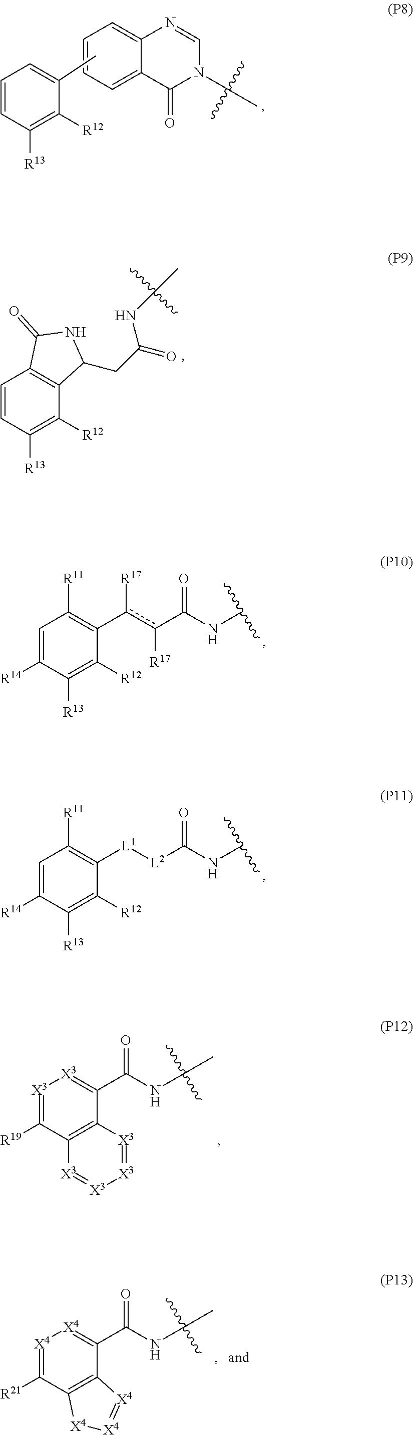 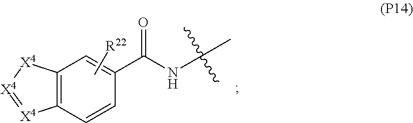
Cyc is C₃₋₆ cycloalkyl;
ring A is independently selected from 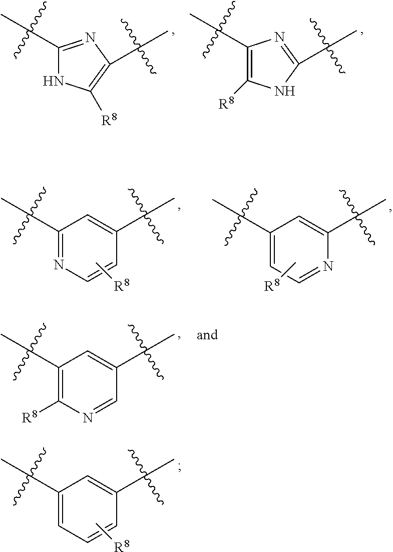
X² is independently selected from N and C⁴;
X³ is independently selected from CR¹⁸ and N;
X⁴ is independently selected from CR²⁰, O, S, N, NH, and. NC₁₋₄alkyl;
L¹ is independently selected from CH₂, O, NH, NC₁₋₄alkyl, and S;
L² is independently selected from NH and NC₁₋₄alkyl;
R¹ and R² are independently selected from H, halogen, C₁₋₄alkyl, and OH;
alternatively, two adjacent R¹ groups form a bond;
R^{3a} is independently selected from H and alkyl;
R^{3b} is independently selected from —(CH₂)ₙ—CN, —(CH₂)ₙ—OR^{b}, —(CH₂)ₙ—OC(═O)NR^{a}R^{a}, —(CH₂)ₙ—NR^{a}R^{a}, —(CH₂)ₙ—C(═O)R^{b}, —(CH₂)ₙ, —C(═O)OR^{b}, —(CH₂)ₙ—NR^{a}C(═O)OR^{b}, —(CH₂)ₙ—NR^{a}C(═O)R^{b}, —NR^{a}C(═O)NR^{a}R^{a}, —C(═O)NR^{a}R^{a};
alternatively, R^{3a} and R^{3b} are taken together to form a heterocyclic ring selected from 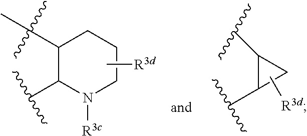
alternatively, R^{3a} and R¹⁰ are taken together to form a heterocyclic ring comprising carbon atoms and 1-3 NR^{3e}, wherein the heterocyclic ring is substituted with R^{3d}, R^{3e} is independently selected from H, C₁₋₄alkyl substituted with 1-5 R⁵, —(CH₂)ₙ—C(═O)R^{b}, —(CH₂)ₙ—C(═O)OR^{b}, —(CH₂)ₙ—C(═O)NR^{a}R^{a}, —(CH₂)ₙ—NHC(═O)OR^{b}, —(CH₂)ₙ—S(═O)ₚR^{e}, —(CH₂)ₙ—S(═O)ₚNR^{a}R^{a}, —(CR_{d}R_{d})ₙ—C₃₋₁₀ carbocyclyl substituted with 1-5 R⁵, and —(CR_{d}R_{d})ₙ-4- to 10-membered heterocyclyl substituted with 1-5 R⁵;
R^{3d} is independently selected from H, ═O, and C₁₋₄ alkyl substituted with 1-5 R⁵;
R⁴ is independently selected from H, halogen, CN, C₁₋₆ alkyl substituted with 1-5 R⁹, and C₃₋₆ cycloalkyl substituted with 1-5 R⁹;
R⁵, at each occurrence, is independently selected from H, D, —(CH₂)ₙ—OR^{b}, ═O, —(CH₂)ₙNH₂, —(CH₂)ₙCN, halogen, C₁₋₆ alkyl, —(CH₂)ₙ—C(═O)OR^{b}, —(CH₂)ₙ-OR^{b}, —(CH₂)ₙ—C₃₋₁₀ carbocyclyl substituted with 0-5 R^{e}, —(CH₂)ₙ-4- to 10-membered heterocyclyl substituted with 0-5 R^{e}, and —O-4- to 10-membered heterocyclyl substituted with 0-5 R^{e};
R⁷ is —(CH₂)ₙNH₂;
R⁸ is independently selected from H, OH, F, Cl, Br, C₁₋₄ alkyl, C₁₋₄alkoxy, CF₃, CN, C₃₋₆ cycloalkyl, aryl, and 5- to 6-membered heterocycle;
R⁹, at each occurrence, is independently selected from H, halogen and OH;
R¹⁰ is independently selected from H, C₁₋₄alkyl, and aryl;
R¹¹ is independently selected from H, halogen, CN, OCH₃, OCF₃, CH₃, C(═O)CH₃, CHF₂, CF₃, CCH₃F₂, OCHF₂, acyl, C₃₋₆ cycloalkyl, and 4-6 membered heterocycle, wherein said aryl, cycloalkyl and heterocycle is optionally substituted with R²⁴;
R¹² is independently selected from H and halogen;
R¹³ is independently selected from H, F, Cl, methyl, ethyl, isopropyl, and OCH₃;
R¹⁴ is independently selected from H, F, and Cl;
R¹⁵ is independently selected from pyrazolyl, aminopyrazolyl, pyrrolidinyl, triazolonyl, aminothiadiazolyl, oxazolyl, thiazolyl, pyridinyl, pyrimidinyl, and 2-pyridonyl;
R¹⁶ is independently selected from H, CN, OR^{b}, halogen, NR^{a}R^{a}, and C₁₋₃ alkyl substituted with 0-5 R^{e};
R¹⁷ is independently selected from H, methyl and halogen;
R¹⁸ is independently selected from H, methyl and halogen;
R¹⁹ is independently selected from C₁₋₄ alkyl, OC₁₋₄ alkyl, SC₁₋₄ alkyl, and C₃₋₆ cycloalkyl;
R²⁰ is independently selected from H, halogen, CN, and C₁₋₄alkyl;
R²¹ is independently selected from C₁₋₄alkyl, OC₁₋₄alkyl, SC₁₋₄alkyl, and C₃₋₆ cycloalkyl;
R²² is independently selected from C₁₋₄alkyl and NR^{a}R^{a};
R²⁴, at each occurrence, is independently selected from H, F, Cl, Br, C(═O)NR^{a}R^{a}, C(═O)OR^{b}, —(CH₂)ₙ—OR^{b}, —(CH₂)ₙ—NR^{a}R^{a}, C₁₋₆ alkyl substituted with 0-5 R^{e}, acyl substituted with 0-5 R^{e}, —(CH₂)ₙ—C₃₋₆ cycloalkyl substituted with 0-5 R^{e}, —(CH₂)ₙ—O-4 to 10-membered heterocyclyl substituted with 0-5 R^{e};
R^{a}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};
R^{b}, at each occurrence, is independently selected from H, C₁₋₆ alkyl substituted with 0-5 R^{e}, C₂₋₆alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e};
R^{c}, at each occurrence, is independently selected from C₁₋₆ alkyl substituted with 0-5 R^{e}, C₂₋₆alkenyl substituted with 0-5 R^{e}, C₂₋₆alkynyl substituted with 0-5 R^{e}, C₃₋₆carbocyclyl, and heterocyclyl;
R^{d}, at each occurrence, is independently selected from H and C₁₋₄alkyl substituted with 0-5 R^{e};
R^{e}, at each occurrence, is independently selected from F, Cl, Br, CN, NO₂, ═O, C₁₋₆ alkyl substituted with 0-5 R^{f}, C₂₋₆ alkenyl, C₂₋₆ alkynyl, —(CH₂)ₙ—C₃₋₆ cycloalkyl, —(CH₂)ₙ-aryl, —(CH₂)ₙ-heterocyclyl, CO₂H, —(CH₂)ₙOR^{f}, SR^{f}, and —(CH₂)ₙ—NR^{f}R^{f};
R^{f}, at each occurrence, is independently selected from H, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl;
n, at each occurrence, is an integer independently selected from 0, 1, 2, and 4; and
p, at each occurrence, is an integer independently selected from 0, 1, and 2.

5. The compound according to claim 4, or a stereoisomer, an enantiomer, a diastereomer, a tautomer, a pharmaceutically acceptable salt thereof, wherein:
Ma is (M2-II): 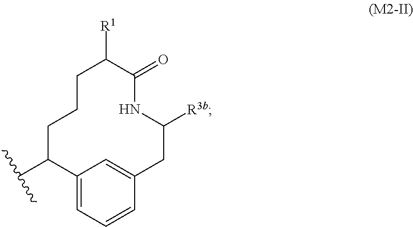
P is independently selected from Formulae (P1), (P2), (P3), (P4), (P6), (P7), (P8),) P(9), P(10), P(11), P(12), P(13), and P(14): 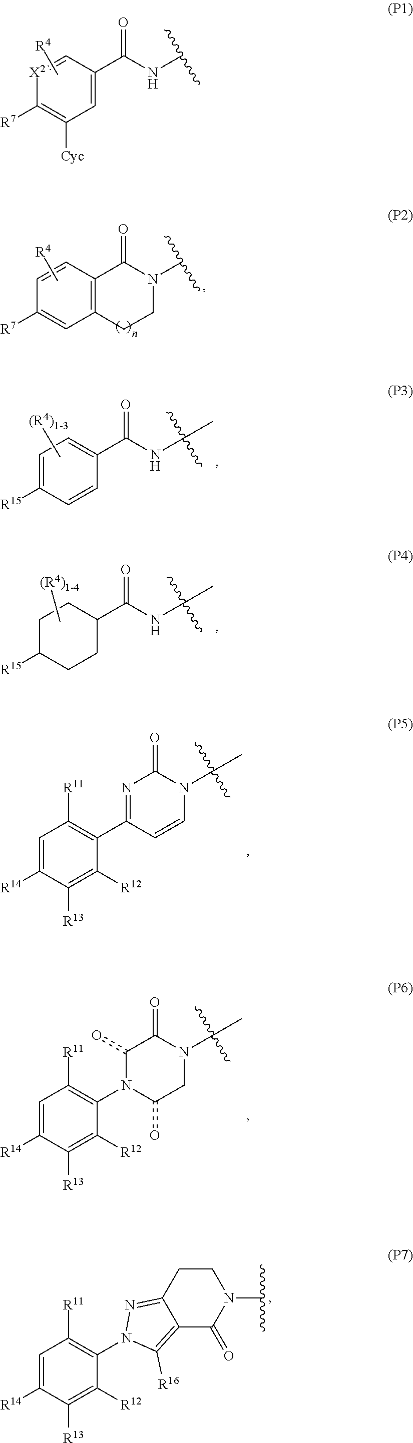 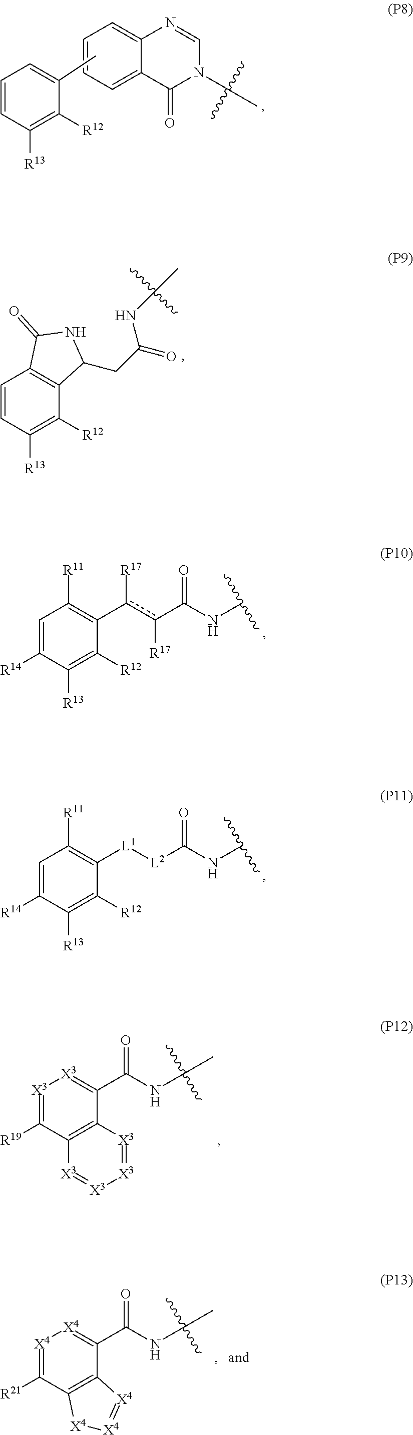 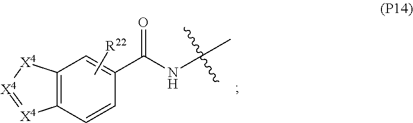
Cyc is C₃₋₆ cycloalkyl;
X² is independently selected from N and CR⁴;
X³ is independently selected from CR¹⁸and N;
X⁴ is independently selected from CR²⁰, O, S, N, NH, and NC¹⁻⁴alkyl;
L¹ is independently selected from CH₂, O, NH, NC₁₋₄alkyl, and S;
L² is independently selected from NH and NC₁₋₄alkyl;
R¹ is independently selected from H and C₁₋₄alkyl;
R^{3b} is independently selected from —(CH₂)ₙ—CN, —(CH₂)ₙ—OR^{b}, —(CH₂)ₙ—OC(═O)NR^{a}R^{a}, —C(═O)OR^{b}, —(CH₂)ₙ—NR^{a}C(═O)OR^{b}, —(CH₂)ₙ—NR^{a}C(═O)R^{b}, —NR^{a}C(═O)NR^{a}R^{a}, and —C(═O)NR^{a}R^{a};
R⁴ is independently selected from H, halogen, CN, C₁₋₆ alkyl substituted with 1-5 R⁹, and C₃₋₆ cycloalkyl substituted with 1-5 R⁹;
R⁵, at each occurrence, is independently selected from H, D, —(CH₂)ₙ—OR^{b}, ═O, —(CH₂)ₙ—NH₂, —(CH₂)ₙCN, halogen, C₁₋₆ alkyl, —(CH₂)ₙ—C(═O)OR^{b}, —(CH₂)ₙ—OR^{b}, —(CH₂)ₙ—C₃₋₁₀ carbocyclyl substituted with 0-5 R^{e}, —(CH₂)ₙ-4- to 10-membered heterocyclyl substituted with 0-5 R^{e}, and —O-4- to 10-membered heterocyclyl substituted with 0-5 R^{e};
R⁷ is —(CH₂)ₙNH₂;
R⁹, at each occurrence, is independently selected from H, halogen and OH;
R¹⁰ is independently selected from H, C₁₋₄alkyl, and aryl;
R¹¹ is independently selected from H, halogen, CN, OCH₃, OCF₃, CH₃, C(═O)CH₃, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴.
R¹² is independently selected from H and halogen;
R¹³ is independently selected from H, F, Cl, methyl, ethyl, isopropyl, and OCH₃;
R¹⁴ is independently selected from H, F, and Cl;
R¹⁵ is independently selected from pyrazolyl, aminopyrazolyl, pyrrolidinyl, triazolonyl, aminothiadiazolyl, oxazolyl, thiazolyl, pyridinyl, pyrimidinyl, and 2-pyridonyl.
R¹⁶ is independently selected from H, CN, OR^{b}, halogen, NR^{a}R^{a}, and C₁₋₃ alkyl substituted with 0-5 R^{e};
R¹⁷ is independently selected from H, methyl and halogen;
R¹⁸ is independently selected from H, methyl and halogen;
R¹⁹ is independently selected from C₁₋₄alkyl, OC₁₋₄ alkyl, SC₁₋₄ alkyl, and C₃₋₆ cycloalkyl;
R²⁰ is independently selected from H, halogen, CN, and C₁₋₄alkyl;
R²¹ is independently selected from C₁₋₄alkyl, OC₁₋₄ alkyl, SC₁₋₄ alkyl, and C₃₋₆ cycloalkyl;
R²² is independently selected from C₁₋₄ alkyl and NR^{a}R^{a};
R²⁴, at each occurrence, is independently selected from H, F, Cl, Br, C(═O)NR^{a}R^{a}, C(═O)OR^{b}, —(CH₂)ₙ—OR^{b}, —(CH₂)ₙ—NR^{a}R^{a}, C₁₋₆ alkyl substituted with 0-5 R^{e}, aryl substituted with 0-5 R^{e}, —(CH₂)ₙ—C₃₋₆ cycloalkyl substituted with 0-5 R^{e}, —(CH₂)ₙ—O-4- to 10-membered heterocyclyl substituted with 0-5 R^{e};
R^{a}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};
R^{b}, at each occurrence, is independently selected from H, C₁₋₆ alkyl substituted with 0-5 R, C₂₋₆ alkenyl substituted with 0-5 R^{e}, C₂₋₆ alkynyl substituted with 0-5 Rₑ, —(CH₂)ₙ—C₃₋₁₀carbocyclyl substituted with 0-5 R^{e}, and —(CH₂)ₙ-heterocyclyl substituted with 0-5 R^{e};
R^{c}, at each occurrence, is independently selected from F, Cl, Br, CN, NO₂, ═O, C₁₋₆ alkyl substituted with 0-5 R^{f}, C₂₋₆ alkenyl, C₂₋₆ alkynyl, —(CH₂)ₙ—C₃₋₆ cycloalkyl, —(CH₂)ₙ-heterocyclyl, CO₂H, —(CH₂)ₙOR^{f}, SR^{f}, and —(CH₂)ₙNR^{f}R^{f}.
R^{f}, at each occurrence, is independently selected from H, C₁₋₅ alkyl optionally substituted with F, Cl, Br, C₃₋₆ cycloalkyl, and phenyl, or R^{f} and R^{f}together with the nitrogen atom to which they are both attached form a heterocyclic ring optionally substituted with C₁₋₄alkyl; and
n, at each occurrence, is an integer independently selected from 0, 1, 2, and 4.

6. The compound according to claim 4, or a stereoisomer, an enantiomer, a diastereomer, a tautomer, a pharmaceutically acceptable salt thereof, wherein:
Ma is (M2-II): 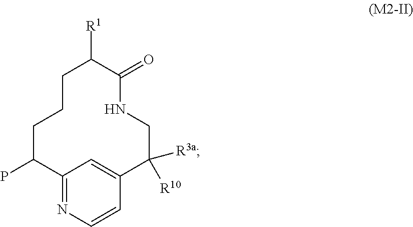
P is independently selected from Formulae (P1), (P2), (P3), (P4), (P5), (P6), (P7), (P8), P(9), P(10), P(11), P(12), P(13), and P(14): 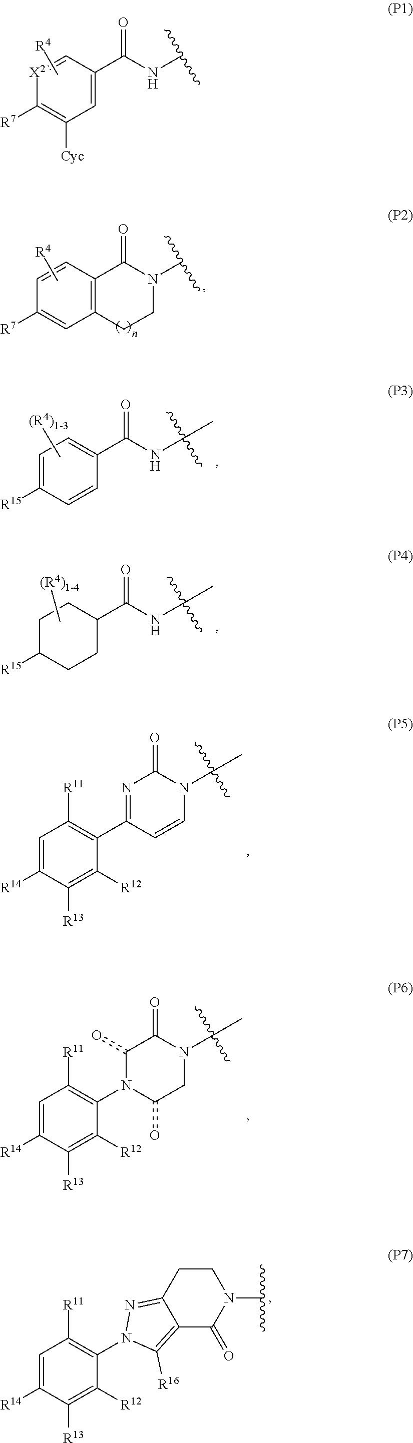 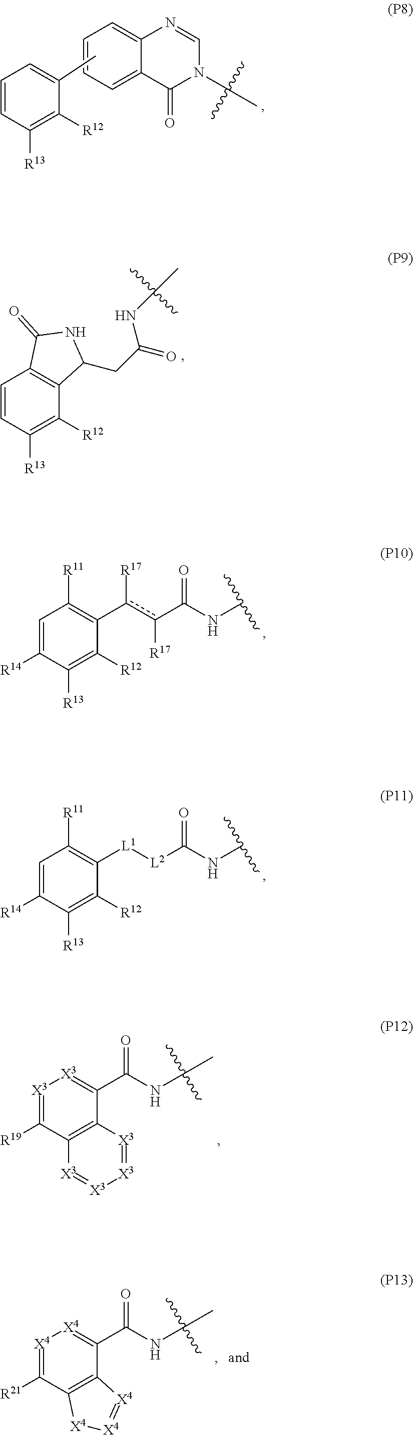 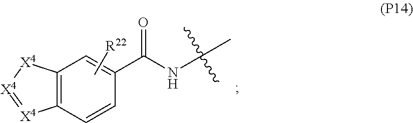
Cyc is C₃₋₆ cycloalkyl;
X² is independently selected from N and CR⁴;
X³ is independently selected from CR¹⁸ and N;
X⁴ is independently selected from CR²⁰, O, S, N, NH, and NC₁₋₄alkyl;
L¹ is independently selected from CH₂, O, NH, NC₁₋₄alkyl, and S;
L² is independently selected from NH and NC₁₋₄alkyl;
R¹ is independently selected from H and C₁₋₄alkyl;
R^{3a} is independently selected from H and C₁₋₄ alkyl;
alternatively, R^{3a} and R¹⁰ are taken together to form 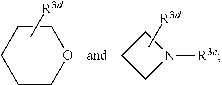
R^{3c} is independently selected from H, C₁₋₄alkyl substituted with 1-5 R⁵, —(CH₂)ₙ—C(═O)R^{b}, —(CH₂)ₙ—C(═O)OR^{b}, —(CH₂)ₙ—C(═O)NR^{a}R^{a}, and —(CH₂)ₙ—NHC(═O)OR^{b};
R^{3d} is independently selected from H and ═O;
R⁴ is independently selected from H, halogen, CN, C₁₋₆ alkyl substituted with 1-5 R⁹, and C₃₋₆ cycloalkyl substituted with 1-5 R⁹;
R⁵, at each occurrence, is independently selected from H, D, —(CH₂)ₙ—OR^{b}, ═O, —(CH₂)ₙNH₂, —(CH₂)ₙCN, halogen, C₁₋₆ alkyl, —(CH₂)ₙ—C(═O)OR^{b}, —(CH₂)ₙ—OR^{b}, —(CH₂)ₙ—C₃₋₁₀ carbocyclyl substituted with 0-5 R^{e}, —(CH₂)ₙ-4- to 10-membered heterocyclyl substituted with 0-5 R^{e}, and —O-4- to 10-membered heterocyclyl substituted with 0-5 R^{e};
R⁶ is independently selected from H, OH, ═O, —(CH₂)ₙNH₂, —(CH₂)ₙCN, halogen, C₁₋₆alkyl, —(CH₂)ₙ—C(═O)OH, —(CH₂)ₙ—C(═O)OC₁₋₄alkyl, —(CH₂)ₙ—OC₁₋₄alkyl, —(CH₂)ₙ—C₃₋₁₀ carbocycle substituted with 0-5 R^{e}, —(CH₂)ₙ-4- to 10-membered heterocycle substituted with 0-5 R^{e}, and —(CH₂)ₙ-4- to 10-membered heterocycle substituted with 0-5 R^{e};
R⁷ is —(CH₂)ₙNH₂;
R⁹, at each occurrence, is independently selected from H, halogen and OH;
R¹⁰ is independently selected from H, C₁₋₄alkyl, and aryl;
R¹¹ is independently selected from H, halogen, CN, OCH₃, OCF₃, CH₃, C(═O)CH₃, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴;
R¹² is independently selected from H and halogen;
R¹³ is independently selected from H, F, Cl, methyl, ethyl, isopropyl, and OCH₃;
R¹⁴ is independently selected from H, F, and Cl;
R¹⁵ is independently selected from pyrazolyl, aminopyrazolyl, pyrrolidinyl, triazolonyl, aminothiadiazolyl, oxazolyl, thiazolyl, pyridinyl, pyrimidinyl, and 2-pyridonyl;
R¹⁶ is independently selected from H, CN, OR^{b}, halogen, NR^{a}R^{a}, and C₁₋₃ alkyl substituted with 0-5 R^{e};
R¹⁷ is independently selected from H, methyl and halogen;
R¹⁵ is independently selected from H, methyl and halogen;
R¹⁹ is independently selected from C₁₋₄alkyl, OC₁₋₄alkyl, SC₁₋₄ alkyl, and C₃₋₆ cycloalkyl;
R²⁰ is independently selected from H, halogen, CN, and C₁₋₄alkyl;
R²¹ is independently selected from C₁₋₄ alkyl, OC₁₋₄alkyl, SC₁₋₄ alkyl, and C₃₋₆ cycloalkyl;
R²² is independently selected from C₁₋₄alkyl and NR^{a}R^{a};
R²⁴, at each occurrence, is independently selected from H, F, Cl, Br, C(═O)NR^{a}R^{a}, C(═O)OR^{b}, —(CH₂)ₙ—OR^{b}, —(CH₂)ₙ—NR^{a}R^{a}, C₁₋₆ alkyl substituted with 0-5 R^{e}, aryl substituted with 0-5 R^{e}, —(CH₂)ₙ—C₃₋₆ cycloalkyl substituted with 0-5 R^{e}, —(CH₂)ₙ—O-4- to 10-membered heterocyclyl substituted with 0-5 R^{e};
R^{a}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)—C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};
R^{b}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e};
R^{c}, at each occurrence, is independently selected from F, Cl, Br, CN, NO₂, ═O, C₁₋₆ alkyl substituted with 0-5 R^{f}, C₂₋₆ alkenyl, C₂₋₆ alkynyl, —(CH₂)ₙ—C₃₋₆ cycloalkyl, —(CH₂)ₙ-aryl, —(CH₂)ₙ-heterocyclyl, CO₂H, —(CH₂)ₙOR^{f}, SR^{f}, and —(CH₂)ₙNR^{f}R^{f};
R^{f}, at each occurrence, is independently selected from H, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl; and
n, at each occurrence, is an integer independently selected from 0, 1, 2, and 4.

7. The compound according to claim 4, or a stereoisomer, an enantiomer, a diastereomer, a tautomer, a pharmaceutically acceptable salt thereof, wherein:
Ma is (M2-III): 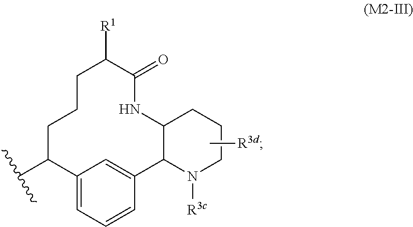
P is independently selected from Formulae (P1), (P2), (P3), (P4), (P5), (P6), (P7), (P8), P(9), P(10), P(11), P(12), P(13), and P(14): 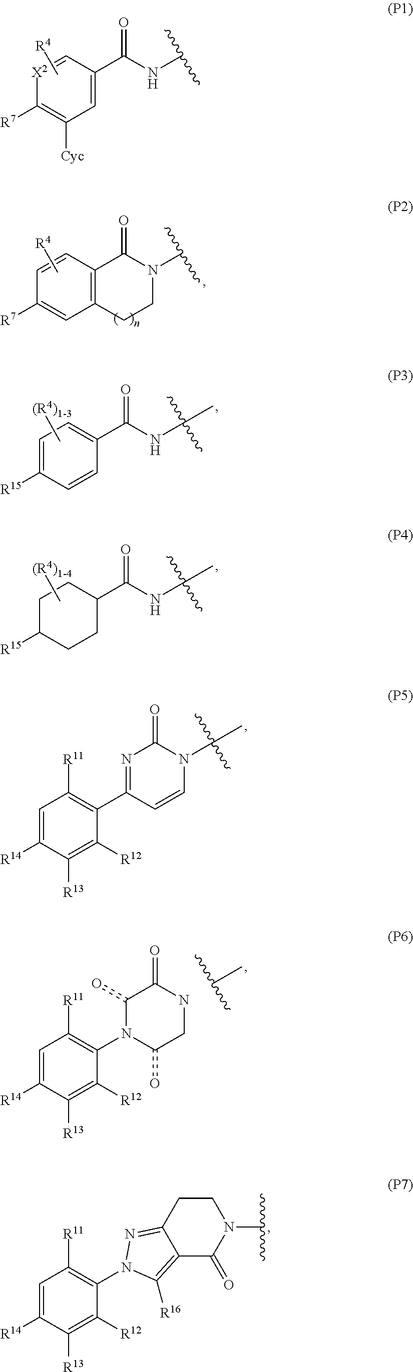 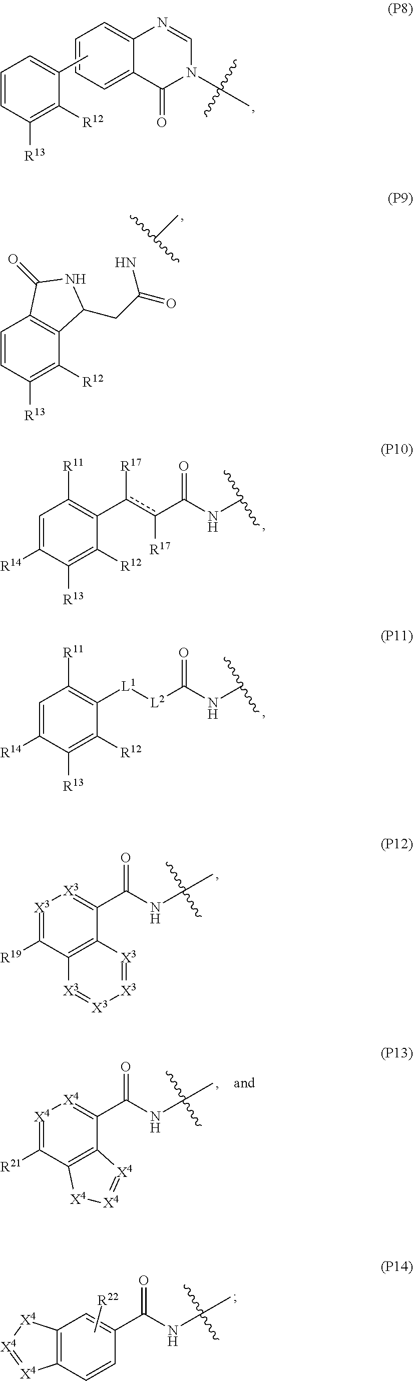
Cyc is C₃₋₆ cycloalkyl;
X²is independently selected from N and CR⁴;
X³ is independently selected from CR¹⁸ and N;
X⁴ is independently selected from CR²⁰, O, S, N, NH, and NC₁₋₄alkyl,
L¹ is independently selected from CH₂, O, NH, NC₁₋₄alkyl, and S;
L² is independently selected from NH and NC₁₋₄alkyl;
R¹ is independently selected from H and C₁₋₄alkyl;
R^{3c} is independently selected from H, C₁₋₄alkyl, —C(═O)C₁₋₄alkyl, —(CH₂)ₙ—C(═O)OH, and —(CH₂)ₙ—C(═O)OC₁₋₄alkyl;
R^{3d} is independently selected from H and ═O;
R⁴ is independently selected from H, halogen, CN, C₁₋₆ alkyl substituted with 1-5 R⁹, and C₃₋₆ cycloalkyl substituted with 1-5 R⁹;
R⁵, at each occurrence, is independently selected from H, D, —(CH₂)ₙ—OR^{b}, ═O, —(CH₂)ₙNH₂, —(CH₂)ₙCN, halogen, C₁₋₆ alkyl, —(CH₂)ₙ—C(═O)OR^{b}, —(CH₂)ₙ—OR^{b}, —(CH₂)ₙ—C₃₋₁₀ carbocyclyl substituted with 0-5 R^{e}, —(CH₂)ₙ-4- to 10-membered heterocyclyl substituted with 0-5 R^{e}, and —O-4- to 10-membered heterocyclyl substituted with 0-5 R^{e};
R⁷ is —(CH₂)ₙNH₂;
R⁹, at each occurrence, is independently selected from ti, halogen and OH;
R¹¹ is independently selected from H, halogen, CN, OCH₃, OCF₃, CH₃, C(═O)CH₃, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴:
R¹² is independently selected from H and halogen;
R¹³ is independently selected from H, F, Cl, methyl, ethyl, isopropyl, and OCH₃;
R¹⁴ is independently selected from H, F, and Cl;
R¹⁵ is independently selected from pyrazolyl, aminopyrazolyl, pyrrolidinyl, triazolonyl, aminothiadiazolyl, oxazolyl, thiazolyl, pyridinyl, pyrimidinyl, and 2-pyridonyl;
R¹⁶ is independently selected from H, CN, OR^{b}, halogen, NR^{a}R^{a}, and C₁₋₃ alkyl substituted with 0-5 R^{e};
R¹⁷ is independently selected from H, methyl and halogen;
R¹⁸ is independently selected from H, methyl and halogen;
R¹⁹ is independently selected from C₁₋₄alkyl, OC₁₋₄alkyl, SC₁₋₄alkyl, and C₃₋₆ cycloalkyl;
R²⁰ is independently selected from H, halogen, CN, and C₁₋₄alkyl;
R²¹ is independently selected from C₁₋₄ alkyl, OC₁₋₄ alkyl, SC₁₋₄ alkyl, and C₃₋₆ cycloalkyl;
R²² is independently selected from C₁₋₄alkyl and NR^{a}R^{a};
R²⁴, at each occurrence, is independently selected from H, F, Cl, Br, C(═O)NR^{a}R^{a}, C(═O)OR^{b}, —(CH₂)ₙ—OR^{b}, —(CH₂)ₙ—NR^{a}R^{a}, C₁₋₆ alkyl substituted with 0-5 R^{e}, aryl substituted with 0-5 R^{e}, —(CH₂)ₙ—C₃₋₆ cycloalkyl substituted with 0-5 R^{e}, —(CH₂)ₙ—O-4- to 10-membered heterocyclyl substituted with 0-5 R^{e};
R^{a}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};
R^{b}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 Rₑ, —(CH₂)ₙ—C₃₋₁₀carbocyclyl substituted with 0-5 R^{e}, and —(CH₂)ₙ-heterocyclyl substituted with 0-5 R^{e};
R^{e}, at each occurrence, is independently selected from F, Cl, Br, CN, NO₂, ═O, C₁₋₆ alkyl substituted with 0-5 R^{f}, C₂₋₆ alkenyl, C₂₋₆ alkynyl, —(CH₂)ₙ—C₃₋₆ cycloalkyl, —(CH₂)ₙ-aryl, —(CH₂)ₙ-heterocyclyl, —(CH₂)ₙOR^{f}, SR^{f}, and —(CH₂)ₙNR^{f}R^{f};
R^{f} at each occurrence, is independently selected from H, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl;
n, at each occurrence, is an integer independently selected from 0, 1, 2, and 4,

8. The compound according to claim 4, or a stereoisomer, an enantiomer, a diastereomer, a tautomer, a pharmaceutically acceptable salt thereof, wherein:
Ma is (M2-IV): 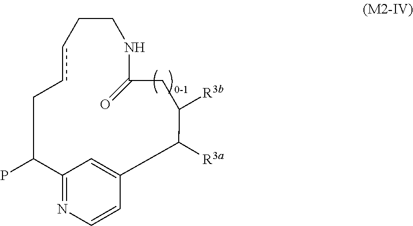
P is independently selected from Formulae (P1), (P2), (P3), (P4), (P5), (P6), (P7), (P8), P(9), P(10), P(11), P(12), P(13), and P(14): 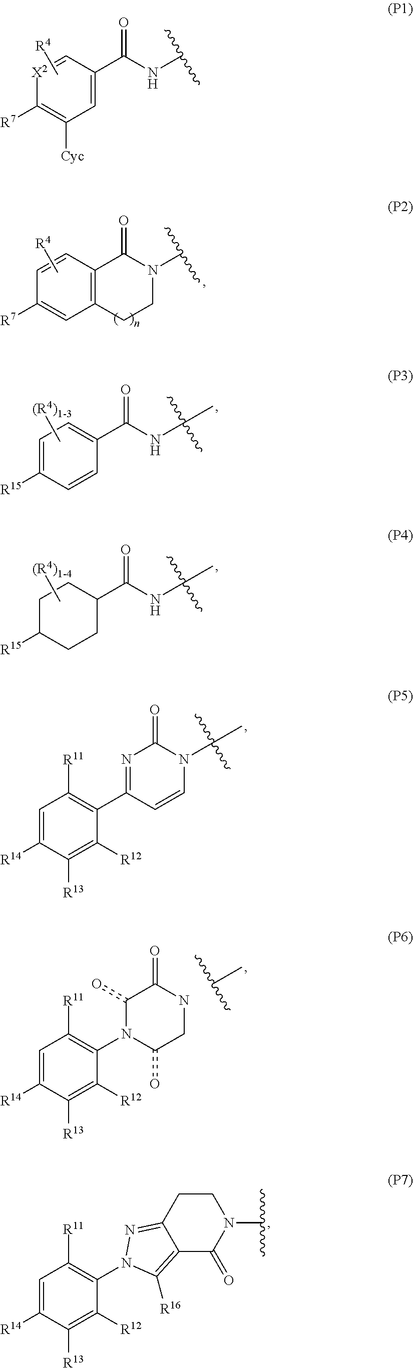 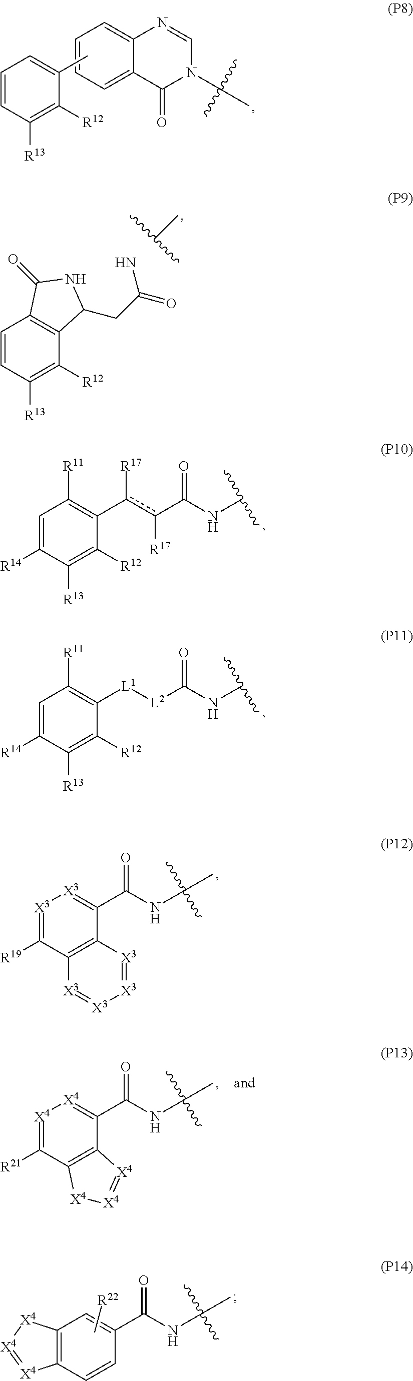
Cyc is C₃₋₆ cycloalkyl;
X² is independently selected from N and CR⁴;
X³ is independently selected from CR¹⁸ and N;
X⁴ is independently selected from CR²⁰, O, S, N, NH, and NC₁₋₄alkyl,
L¹ is independently selected from CH₂, O, NH, NC₁₋₄alkyl, and S;
L² is independently selected from NH and NC₁₋₄alkyl;
R¹ is independently selected from H and C₁₋₄alkyl;
R^{3a} is H.
R^{3b} is H;
alternatively, R^{3a} and R^{3b} are taken together to form 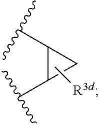
R^{3d} is independently selected from H and ═O;
R⁴ is independently selected from H, halogen, CN, C₁₋₆ alkyl substituted with 1-5 R⁹, and C₃₋₆ cycloalkyl substituted with 1-5 R⁹;
R⁵, at each occurrence, is independently selected from H, D, —(CH₂)ₙ—OR^{b}, ═O, —(CH₂)ₙNH₂, —(CH₂)ₙ—CN, halogen, C₁₋₆ alkyl, —(CH₂)ₙ—C(═O)OR^{b}, —(CH₂)ₙ—OR^{b}, —(CH₂)ₙ—C₃₋₁₀ carbocyclyl substituted with 0-5 R^{e}, —(CH₂)ₙ-4- to 1.0-membered heterocyclyl substituted with 0-5 R^{e}, and —O-4- to 10-membered heterocyclyl substituted with 0-5 R^{e};
R⁷ is —(CH₂)ₙNH₂;
R⁹, at each occurrence, is independently selected from H, halogen and OH;
R¹¹ is independently selected from H, halogen, CN, OCH₃, OCF₃, CH₃, C(═O)CH₂, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴;
R¹² is independently selected from H and halogen;
R¹³ is independently selected from H, F, Cl, methyl, ethyl, isopropyl, and OCH₃;
R¹⁴ is independently selected from H, F, and Cl;
R¹⁵ is independently selected from pyrazolyl, aminopyrazolyl, pyrrolidinyl, triazolonyl, aminothiadiazolyl, thiazolyl, pyridinyl, pyrimidinyl, and 2-pyridonyl;
R¹⁶ is independently selected from H, CN, OR^{b}, halogen, NR^{a}R^{a}, and C₁₋₃ alkyl substituted with 0-5 R^{e};
R¹⁷ is independently selected from H, methyl and halogen;
R¹⁸ is independently selected from H, methyl and halogen;
R¹⁹ is independently selected from C₁₋₄alkyl, OC₁₋₄ alkyl, SC₁₋₄alkyl, and C₃₋₆ cycloalkyl;
R²⁰ is independently selected from H, halogen, CN, and C₁₋₄ alkyl;
R²¹ is independently selected from C₁₋₄alkyl, OC₁₋₄ alkyl, SC₁₋₄ alkyl, and C₃₋₆ cycloalkyl;
R²² is independently selected from C₁₋₄alkyl and NR^{a}R^{a};
R²⁴ , at each occurrence, is independently selected from H, F, Cl, Br, C(═O)NR^{a}R^{a}, C(O)OR^{b}, —(CH₂)ₙ—OR^{b}, —(CH₂)ₙ—NR^{a}R^{a}, C₁₋₆ alkyl substituted with 0-5 R^{e}, aryl substituted with 0-5 R^{e}, —(CH₂)ₙ—C₃₋₆ cycloalkyl substituted with 0-5 R^{e}, —(CH₂)ₙ—O-4 to 10-membered heterocyclyl substituted with 0-5 R^{e};
R^{a}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ-C₃₋₁₀carbocyclyl substituted with 0-5 R^{e}, and —(CH₂)ₙ-heterocyclyl substituted. with 0-5 R^{e}; or R^{a} and R^{a} together with the nitrogen atom to which they are bath attached form a heterocyclic ring substituted with 0-5 R^{e};
R^{b}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e};
R^{e}, at each occurrence, is independently selected from F, Cl, Br, CN, NO₂, ═O, C₁₋₆ alkyl substituted with 0-5 R^{f}, C₂₋₆ alkenyl, C₂₋₆ alkynyl, —(CH₂)ₙ—C₃₋₆ cycloalkyl, —(CH₂)ₙ-aryl, —(CH₂)ₙ-heterocyclyl, CO₂H, —(CH₂)ₙOR^{f}, SR^{f}, and —(CH₂)ₙNR^{f}R^{f}.
R^{f}, at each occurrence, is independently selected from H, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl;
n, at each occurrence, is an integer independently selected from 0, 1, 2, and 4.

9. The compound according to claim 1, or a stereoisomer, an enantiomer, a diastereomer, a tautomer, a pharmaceutically acceptable salt thereof, wherein:
Ma is independently selected from Formula (M3): 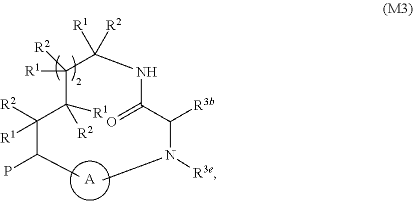
P is independently selected from Formulae (P1), (P2), (P3), (P4), (P5), (P6), (P7), (P8), P(9), P(10), P(11), P(12), P(13), and P(14): 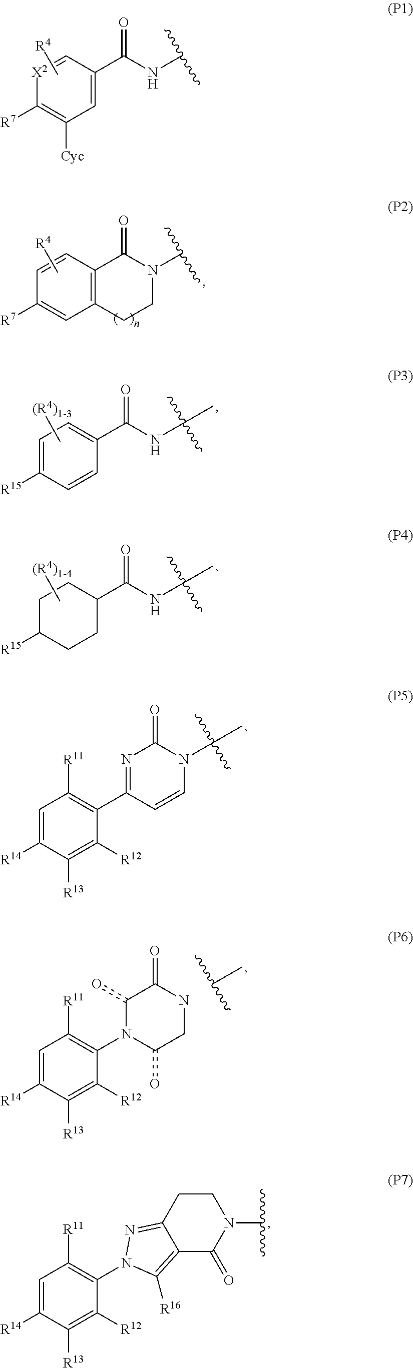 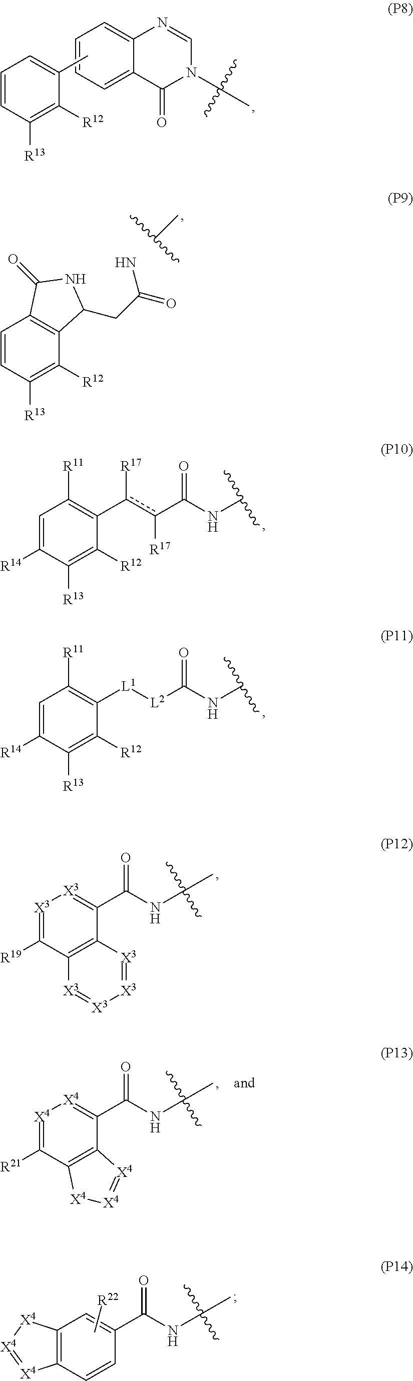
Cyc is C₃₋₆ cycloalkyl;
ring A is independently selected from 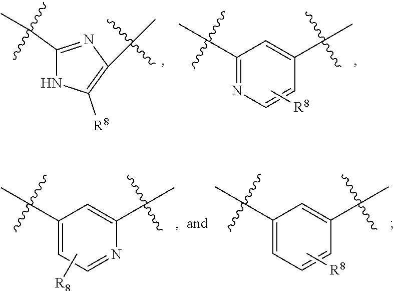
X² is independently selected from N and CR⁴;
X³ is independently selected from CR¹⁸ and N;
X⁴ is independently selected from CR²⁰, O, S, N, NH, and NC₁₋₄alkyl;
L¹ is independently selected from CH₂, O, NH, NC₁₋₄alkyl, and S;
L² is independently selected from NH and NC₁₋₄alkyl;
R¹ and R² are independently selected from H and C₁₋₄alkyl;
alternatively, two adjacent R¹ groups form a bond;
R^{3b} is independently selected from —(CH₂)ₙ—NR^{a}R^{a}, —(CH₂)ₙ—C(═O)R^{b}, —(CH₂)ₙ—C(═O)OR^{b}, —(CH₂)ₙ—NR^{a}C(═O)OR^{b}, —(CH₂)ₙNR^{a}C(═O)R^{b}, —NR^{a}C(═O)NR^{a}R^{a}, —C(═O)NR^{a}R^{a};
R^{3c} is independently selected from H and C₁₋₄alkyl;
alternatively, R^{3b} and R^{3c} are taken together to form a heterocyclic ring selected from 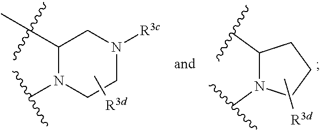
R^{3c} is independently selected from H, C₁₋₄alkyl substituted with 1-5 R⁵, —(CH₂)ₙ—C(═O)R^{b}, —(CH₂)ₙ—C(═O)OR^{b}, —(CH₂)ₙ—C(═O)NR^{a}R^{a}, —(CH₂)ₙ—NHC(═O)OR^{b}, —(CH₂)ₙ—S(═O)ₚR^{c}, —(CH₂)ₙ—S(═O)ₚNR^{a}R^{a}, —(CR_{d}R_{d})ₙ—C₃₋₁₀ carbocyclyl substituted with 1-5 R⁵, and —(CR_{d}R_{d})ₙ-4- to 10-membered heterocyclyl substituted with 1-5 R⁵;
R^{3d} is independently selected from H, ═O, and C₁₋₄ alkyl substituted with 1-5 R⁵;
R⁴ is independently selected from H, halogen, CN, C₁₋₆ alkyl substituted with 1-5 R⁹, and C₃₋₆ cycloalkyl substituted with 1-5 R⁹;
R⁵, at each occurrence, is independently selected from H, D, —(CH₂)ₙ—OR^{b}, ═O, —(CH₂)ₙNH₂, —(CH₂)ₙCN, halogen, C₁₋₆ alkyl, —(CH₂)ₙ—C(═O)OR^{b}, —(CH₂)OR^{b}, —(CH₂)ₙ—C₃₋₁₀ carbocyclyl substituted with 0-5 R^{e}, —(CH₂)ₙ-4- to 10-membered heterocyclyl substituted with 0-5 R^{e}, and —O-4- to 10-membered heterocyclyl substituted with 0-5 R^{e};
R⁷ is —(CH₂)ₙNH₂;
R⁸ is independently selected from H, OH, F, Cl, Br, C₁₋₄ alkyl. C₁₋₄alkoxy, CF₃, CN, C₃₋₆ cycloalkyl, aryl, and 5- to 6-membered heterocycle,
R⁹, at each occurrence, is independently selected from H, halogen and OH;
R¹¹ is independently selected from H, halogen, CN, OCH₃, OCF₃, CH₃, C(═O)CH₃, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴;
R¹² is independently selected from H and halogen;
R¹³ is independently selected from H, F, Cl, methyl, ethyl, isopropyl, and OCH₃;
R¹⁴ is independently selected from H, F, and Cl;
R¹⁵ is independently selected from pyrazolyl, aminopyrazolyl, pyrrolidinyl, triazolonyl, aminothiadiazolyl, oxazolyl, thiazolyl, pyridinyl, pyrimidinyl, and 2-pyridonyl;
R¹⁶ is independently selected from H, CN, OR^{b}, halogen, NR^{a}R^{a}, and C₁₋₃ alkyl substituted with 0-5 R^{e};
R¹⁷ is independently selected from H, methyl and halogen;
R¹⁸ is independently selected from H, methyl and halogen;
R¹⁹ is independently selected from C₁₋₄alkyl, OC₁₋₄ alkyl, SC₁₋₄alkyl, and C₃₋₆ cycloalkyl;
R²⁰ is independently selected from H, halogen, CN, and C₁₋₄alkyl;
R²¹ is independently selected from C₁₋₄ alkyl, OC₁₋₄ alkyl, SC₁₋₄ alkyl, and C₃₋₆ cycloalkyl;
R²² is independently selected from C₁₋₄alkyl and NR^{a}R^{a};
R²⁴, at each occurrence, is independently selected from H, F, Cl, Br, C(═O)NR^{a}R^{a}, C(═O)OR^{b}, —(CH₂)ₙ—OR^{b}, —(CH₂)ₙ—NR^{a}R^{a}, C₁₋₆ alkyl substituted with 0-5 R^{e}, aryl substituted with 0-5 R^{e}, —(CH₂)ₙ—C₃₋₆ cycloalkyl substituted with 0-5 R^{e}, —(CH₂)ₙ—O-4- to 10-membered heterocyclyl substituted with 0-5 R^{e};
R^{a}, at each occurrence, is independently selected from H, C₁₋₆ alkyl substituted with 0-5 R^{e}, C₂₋₆ alkenyl substituted with 0-5 R^{e}, C₂-c alkynyl substituted with 0-5 R^{e}, —(CH₂)ₙ-C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e}; R^{b}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e};
R^{c}, at each occurrence, is independently selected from C₁₋₆ alkyl substituted with 0-5 R^{e}, C₂₋₆alkenyl substituted with 0-5 R^{e}, C₂₋₆alkynyl substituted with 0-5 R^{e}, C₃₋₆-carbocyclyl, and heterocyclyl;
R^{d}, at each occurrence, is independently selected from H and C₁₋₄alkyl substituted with 0-5 R^{e};
R^{e}, at each occurrence, is independently selected from F, Cl, Br, CN, NO₂, ═O, C₁₋₆ alkyl substituted with 0-5 R^{f}, C₂₋₆ alkenyl, C₂₋₆ alkynyl, —(CH₂)ₙ—C₃₋₆ cycloalkyl, —(CH₂)ₙ-aryl, —(CH₂)ₙ-heterocyclyl, CO₂H, —(CH₂)ₙOR^{f}, SR^{f}, and —(CH₂)ₙNR^{f}R^{f};
R^{f}, at each occurrence, is independently selected from H, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl;
n, at each occurrence, is an integer independently selected from 0, 1. 2, and 4; and
p, at each occurrence, is an integer independently selected from 0, 1, and 2.

10. The compound according to claim 9, or a stereoisomer, an enantiomer, a diastereomer, a tautomer, a pharmaceutically acceptable salt thereof, wherein:
Ma is (M3-I): 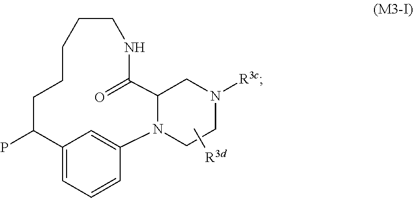
P is independently selected from Formulae (P1), (P2), (P3), (P4), P5), P6 (P7), (P8), P(9), P(10), P(11), P(12), P(13), and P(14): 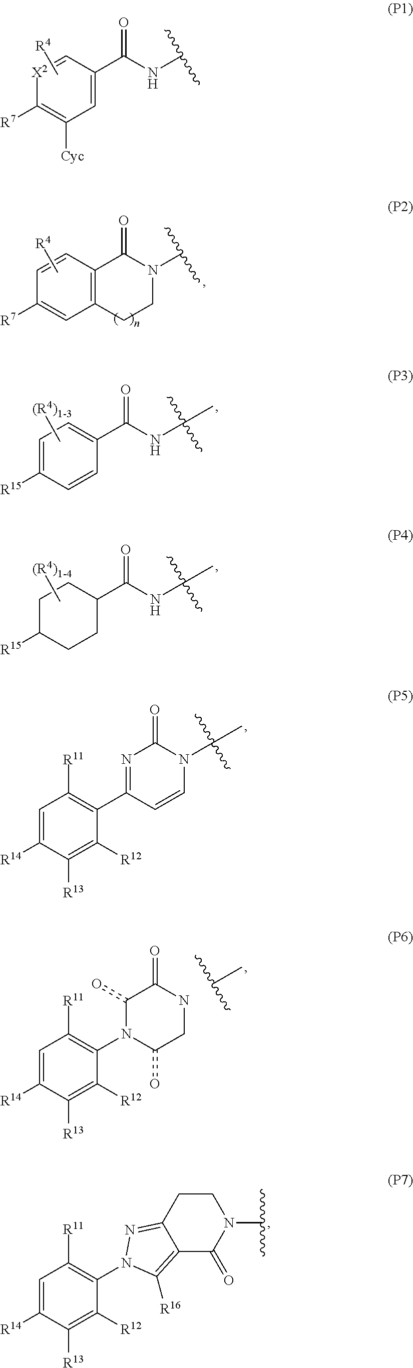 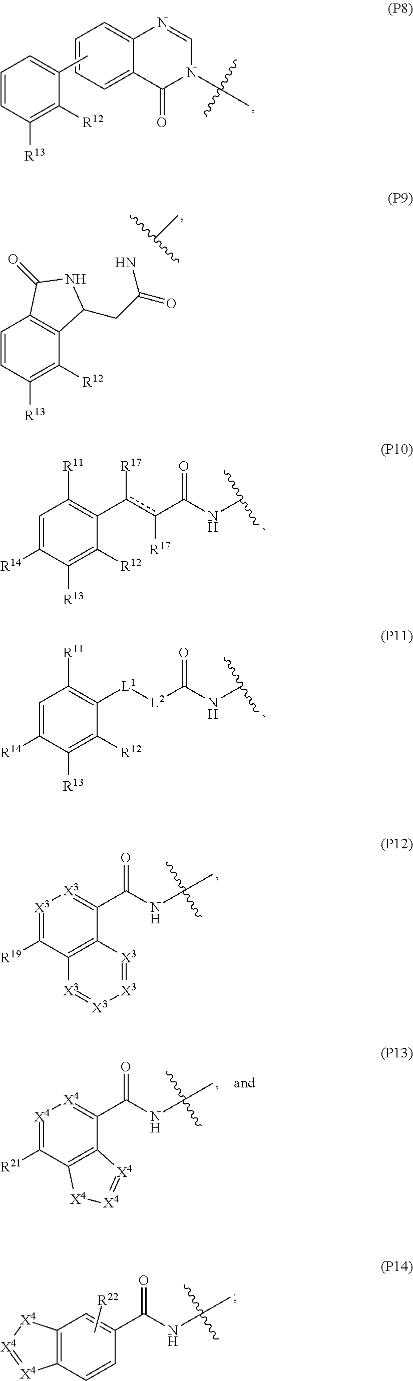
Cyc is C₃₋₆cycloalkyl;
X² is independently selected from N and CR⁴;
X³ is independently selected from CR¹⁸ and N;
X⁴ is independently selected from CR²⁰, O, S, N, NH, and NC₁₋₄alkyl;
L¹ is independently selected from CH₂, O, NH, NC₁₋₄alkyl, and S;
L² is independently selected from NH and NC₁₋₄alkyl;
R^{3c} is independently selected from H, C₁₋₄alkyl, —(CH₂)ₙ—C(═O)R^{b}, —(CH₂)ₙ—C(═O)OR^{b}, —(CH₂)ₙ—C(═O)NR^{a}R^{a}, —(CH₂)ₙ—S(═O)ₚR^{c}, —S(═O)ₚNR^{a}R^{a}, —(CH₂)ₙ—C₃₋₁₀ carbocyclyl substituted with 1-5 R⁵, and —(CR_{d}R_{d})ₙ-4- to 10-membered heterocyclyl substituted with 1-5 R⁵;
R^{3d} is independently selected from H and ═O;
R⁵, at each occurrence, is independently selected from H, D, —(CH₂)ₙ—OR^{b}, ═O, —(CH₂)ₙNH₂, —(CH₂)ₙCN, halogen, C₁₋₆alkyl, —(CH₂)ₙ—C(═O)OR^{b}, —(CH₂)ₙ—OR^{b}, —(CH₂)ₙ—C₃₋₁₀ carbocyclyl substituted with 0-5 R^{e}, —(CH₂)ₙ-4- to 10-membered heterocyclyl substituted with 0-5 R^{e}, and —O-4- to 10-membered heterocyclyl substituted with 0-5 R^{e};
R⁷ is —(CH₂)ₙNH₂;
R⁸ is independently selected from H, OH, F, Cl, Br, C₁₋₄alkyl, C₁₋₄alkoxy, CF₃, CN, C₃₋₆ cycloalkyl, aryl, and 5- to 6-membered heterocycle;
R⁹, at each occurrence, is independently selected from H, halogen and OH;
R¹¹ is independently selected from H, halogen, CN, OCH₃, OCF₃, CH₃, C(═O)CH₃, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴;
R¹² is independently selected from H and halogen;
R¹³ is independently selected from H, F, Cl, methyl, ethyl, isopropyl, and OCH₃;
R¹⁴ is independently selected from H, F, and Cl;
R¹⁵ is independently selected from pyrazolyl, aminopyrazolyl, pyrrolidinyl, triazolonyl, aminothiadiazolyl, oxazolyl, thiazolyl, pyridinyl, pyrimidinyl, and 2-pyridonyl;
R¹⁶ is independently selected from H, CN, OR^{b}, halogen, NR^{a}R^{a}, and C₁₋₃ alkyl substituted with 0-5 R^{e};
R¹⁷ is independently selected from H, methyl and halogen;
R¹⁸ is independently selected from H, methyl and halogen;
R¹⁹ is independently selected from C₁₋₄ alkyl, OC₁₋₄alkyl, SC₁₋₄alkyl, and C₃₋₆ cycloalkyl;
R²⁰ is independently selected from H, halogen, CN, and C₁₋₄alkyl;
R²¹ is independently selected from C₁₋₄ alkyl, OC₁₋₄ alkyl, SC₁₋₄ alkyl, and C₃₋₆ cycloalkyl;
R²² is independently selected from C₁₋₄alkyl and NR^{a}R^{a};
R²⁴, at each occurrence, is independently selected from H, F, Cl, Br, C(═O)NR^{a}R^{a}, C(═O)OR^{b}, —(CH₂)ₙ—OR^{b}, —(CH₂)ₙ—NR^{a}R^{a}, C₁₋₆ alkyl substituted with 0-5 R^{e}, aryl substituted with 0-5 R^{e}, —(CH₂)ₙ—C₃₋₆ cycloalkyl substituted with 0-5 R^{e}, —(CH₂)ₙ—O-4- to 10-membered heterocyclyl substituted with 0-5 R^{e},
R^{a}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};
R^{b}, at each occurrence, is independently selected from H, C₁₋₆ alkyl substituted with 0-5 R^{e}, C₂ alkenyl substituted with 0-5 R^{e}, C₂₋₆ alkynyl substituted with 0-5 Rₑ, —(CH₂)ₙ—C₃₋₁₀carbocyclyl substituted with 0-5 R^{e}, and —(CH₂)ₙ-heterocyclyl substituted with 0-5 R^{e};
R^{c}, at each occurrence, is independently selected from C₁₋₆ alkyl substituted with 0-5 R^{e}, C₂₋₆alkenyl substituted with 0-5 R^{e}, C₂₋₆alkynyl substituted with 0-5 R^{e}, C₃₋₆carbocyclyl, and heterocyclyl;
R^{d}, at each occurrence, is independently selected from H and C₁₋₄alkyl substituted with 0-5 R^{e};
R^{e}, at each occurrence, is independently selected from F, Cl, Br, CN, NO₂, ═O, C₁₋₆ alkyl substituted with 0-5 R^{f}, C₁₋₆ alkenyl, C₁₋₆ alkynyl, —(CH₂)ₙ—C₃₋₆ cycloalkyl, —(CH₂)ₙ-aryl, —(CH₂)ₙ-heterocyclyl, CO₂H, —(CH₂)ₙOR^{f}, SR^{f}, and —(CH₂)ₙNR^{f}R^{f};
R^{f}, at each occurrence, is independently selected from H, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C¹⁻⁴alkyl;
n, at each occurrence, is an integer independently selected from 0, 1, 2, and 4, and
p, at each occurrence, is an integer independently selected from 0, 1, and 2.

11. The compound according to claim 1, or a stereoisomer, an enantiomer, a diastereomer, a tautomer, a pharmaceutically acceptable salt thereof, wherein:
Ma is independently selected from Formula (M4): 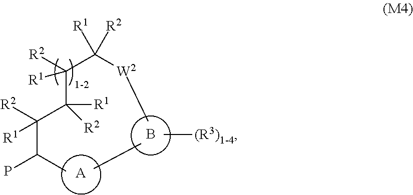
P is independently selected from Formulae (P1), (P2), (P3), (P4), (P5), (P6), (P7), (P8). P(9), P(10), P(11), P(12), P(13), and P(14): 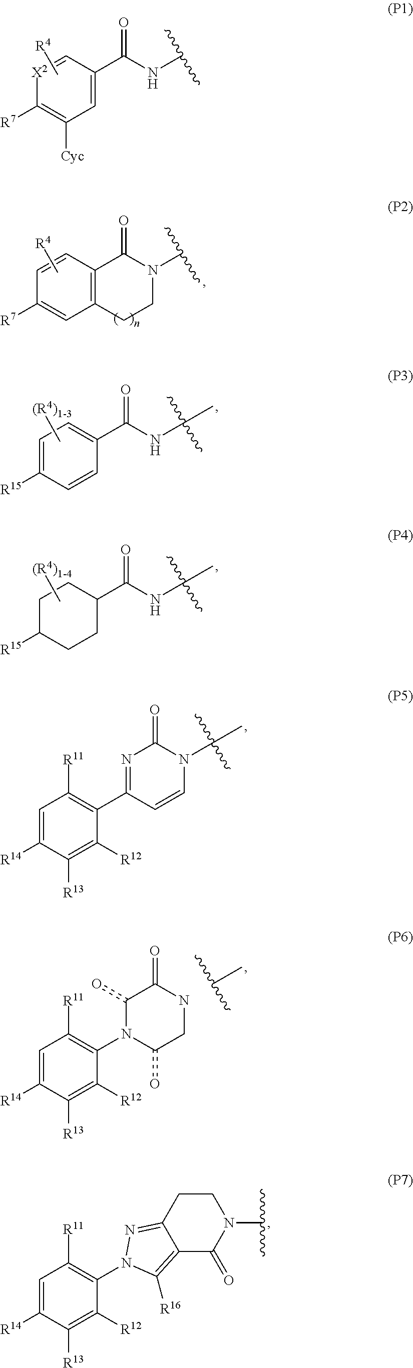 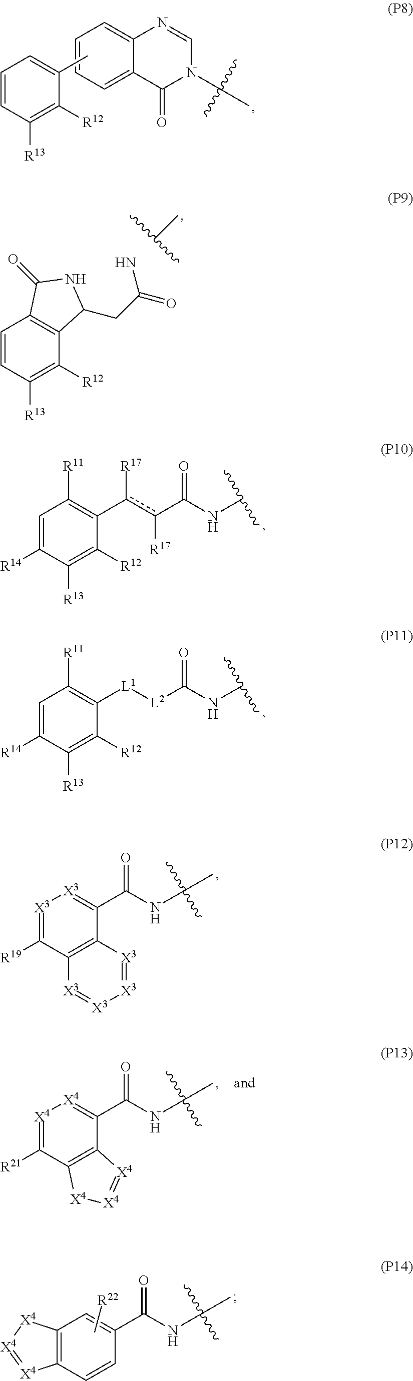
Cyc is C₃₋₆ cycloalkyl;
ring A is independently selected from 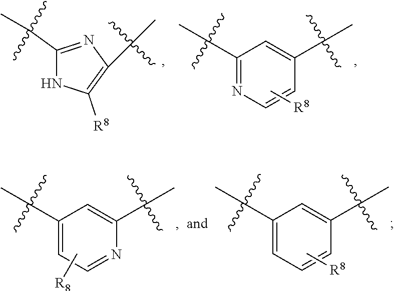
ring B is independently selected from 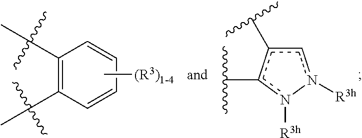
W² is independently selected from —C(O)NH, —NHC(O)—, and —NHC(O)CH₂—;
X² is independently selected from N and CR⁴;
X³ is independently selected from CR¹⁸ and N;
X⁴ is independently selected from CR²⁰, O, S, N, NH, and NC₁₋₄alkyl;
L¹ is independently selected from CH₂, O, NH, NC₁₋₄alkyl, and S;
L² is independently selected from NH and NC₁₋₄alkyl;
R¹ and R² are independently selected from H and C₁₋₄ alkyl;
R³ is independently selected from H, ═O, halogen, C₁₋₄ alkyl substituted with 1-5 R⁵, CN, —OR^{b}, —NR^{a}R^{a}, —C(═O)R^{b}, —C(═O)OR^{b}, —NR^{a}C(═O)OR^{b}, —NR^{a}C(═O)R^{b}, —NR^{a}C(═O)NR^{a}R^{a}, —C(═O)NR^{a}R^{a}, —S(═O)ₚR^{c}, —(CH₂)ₙ—C₃₋₁₀carbocyclyl substituted with 1-5 R⁵, and —(CH₂)ₙ-4- to 10-membered heterocyclyl substituted with 1-5 R⁵;
R^{h} is absent or independently selected from H, C₁₋₄-alkyl substituted with 1-4 R⁵, —(CH₂)ₙ—C(═O)R^{b}, —(CH₂)ₙ—C(═O)OR^{b};
R⁴ is independently selected from H, halogen, CN, C₁₋₆ alkyl substituted with 1-5 R⁹, and C₃₋₆ cycloalkyl substituted with 1-5 R⁹;
R⁵, at each occurrence, is independently selected from H, D, —OR^{b}, —NH₂, —CN, halogen, C₁₋₆alkyl, —C(═O)OR^{b}, C₃₋₁₀ carbocyclyl substituted with 0-5 R^{e}, -4- to 10-membered heterocyclyl substituted with 0-5 R^{e}, and —O-4- to 10-membered heterocyclyl substituted with 0-5 R^{e};
R⁷ is —(CH₂)ₙNH₂;
R⁸ is independently selected from H, OH, F, Cl, Br, C₁₋₄ alkyl, C₁₋₄ alkoxy, CF₃, CN, C₃₋₆ cycloalkyl, aryl, and 5- to 6-membered heterocycle;
R⁹, at each occurrence, is independently selected from H, halogen and OH;
R¹¹ is independently selected from H, halogen, CN, OCH₃, OCF₃, CH₃, C(═O)CH₃, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴;
R¹² is independently selected from H and halogen;
R¹³ is independently selected from H, F, Cl, methyl, ethyl, isopropyl, and OCH₃;
R¹⁴ is independently selected from H, F, and Cl;
R¹⁵ is independently selected from pyrazolyl, aminopyrazolyl, pyrrolidinyl, triazolonyl, aminothiadiazolyl, oxazolyl, thiazolyl, pyridinyl, pyrimidinyl, and 2-pyridonyl;
R¹⁶ is independently selected from H, CN, OR^{b}, halogen, NR^{a}R^{a}, and C₁₋₃ alkyl substituted with 0-5 R^{e};
R¹⁷ is independently selected from H, methyl and halogen;
R¹⁸ is independently selected from H, methyl and halogen;
R¹⁹ is independently selected from C₁₋₄alkyl, OC₁₋₄ alkyl, SC₁₋₄ alkyl, and C₃₋₆ cycloalkyl;
R²⁰ is independently selected from H, halogen, CN, and C₁₋₄ alkyl;
R²¹ is independently selected from C₁₋₄alkyl, OC₁₋₄ alkyl, SC₁₋₄ alkyl, and C₃₋₆ cycloalkyl;
R²² is independently selected from C₁₋₄alkyl and NR^{a}R^{a};
R²⁴, at each occurrence, is independently selected from H, F, Cl, Br, C(═O)NR^{a}R^{a}, C(═O)OR^{b}, —(CH₂)ₙ—OR^{b}, —(CH₂)ₙ—NR^{a}R^{a}, C₁₋₆ alkyl substituted with 0-5 R^{e}, aryl substituted with 0-5 R^{e}, —(CH₂)ₙ—C₃₋₆ cycloalkyl substituted with 0-5 R^{e}, —(CH₂)ₙ—O-4- to 10-membered heterocyclyl substituted with 0-5 R^{e};
R^{a}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};
R^{b}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 Rₑ, —(CH₂)₁—C₃₋₁₀carbocyclyl substituted with 0-5 R^{e}, and —(CH₂)ₙ-heterocyclyl substituted with 0-5 R^{e}.
R^{c}, at each occurrence, is independently selected from C₁₋₆ alkyl substituted with 0-5 R^{e}, C₂₋₆alkenyl substituted with 0-5 R^{e}, C₂₋₆alkynyl substituted with 0-5 R^{e}, C₃₋₆carbocyclyl, and heterocyclyl;
R^{d}, at each occurrence, is independently selected from H and C₁₋₄alkyl substituted with 0-5 R^{e};
R^{e}, at each occurrence, is independently selected from F, Cl, Br, CN, NO₂, ═O, C₁₋₆ alkyl substituted with 0-5 R^{f}, C₂₋₆ alkenyl, C₂₋₆ alkynyl, —(CH₂)ₙ—C₃₋₆ cycloalkyl, —(CH₂)ₙ-heterocyclyl, CO₂H, —(CH₂)ₙOR^{f}, SR^{f}, and —(CH₂)ₙNR^{f}R^{f};
R^{f}, at each occurrence, is independently selected from H, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl;
n, at each occurrence, is an integer independently selected from 0, 1, 2, and 4; and
p, at each occurrence, is an integer independently selected from 0, 1, and 2.

12. The compound according to claim 11 or a stereoisomer, an enantiomer, a diastereomer, a tautomer, a pharmaceutically acceptable salt thereof, wherein:
Ma is (M4-I): 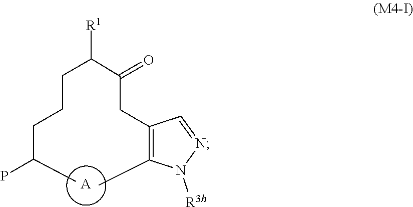
P is independently selected from Formulae P1), (P2), (P3), (P4), (P5), (P6), (P7), (P8), P(9), P(10), P(11), P(12), P(13), and P(14): 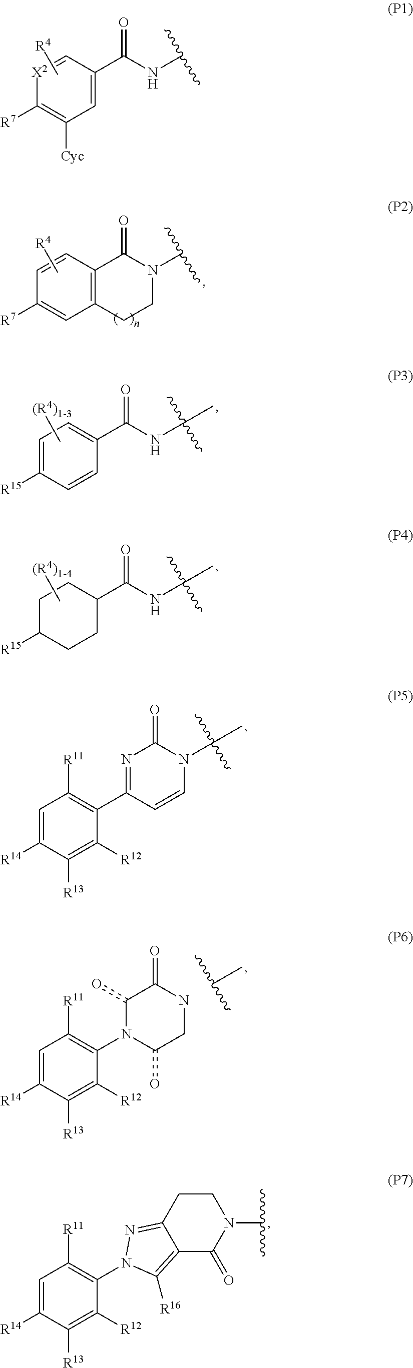 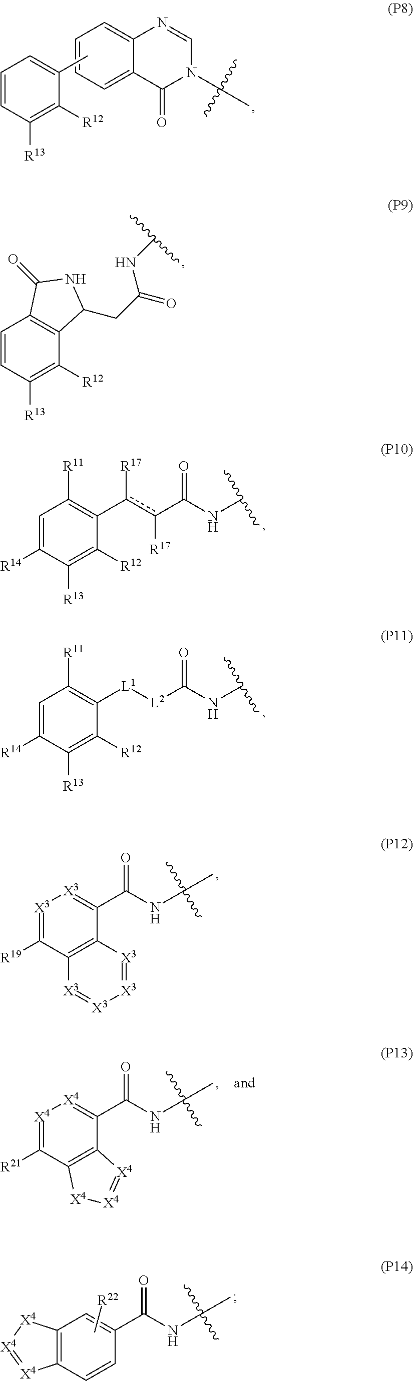
Cyc is C₃₋₆ cycloalkyl;
ring A is independently selected from 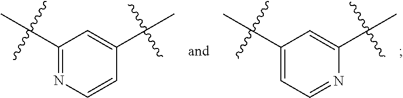 [SMILES: *c1ccnc(*)c1 ; *c1ccnc(*)c1]
X² is independently selected from N mid CR⁴;
X³ is independently selected from CR¹⁸ and N;
X⁴ is independently selected from CR²⁰, O, S. N, NH, and NC₁₋₄alkyl;
L¹ is independently selected from CH₂, O, NH, NC₁₋₄alkyl, and S;
L² is independently selected from NH and NC₁₋₄alkyl;
R¹ is independently selected from H and C₁₋₄alkyl;
R^{3h} is independently selected from H, CHF₂, CD₃, and CH₃;
R⁷ is —(CH₂)ₙNH₂;
R⁹, at each occurrence, is independently selected from H, halogen and OH;
R¹¹ is independently selected from H, halogen, CN, OCH₃, OCF₃, CH₃, C(═O)CH₃, CHF₂, CF₃, CCH₃F₂, OCHF₂, aryl, C₃₋₆ cycloalkyl, and 4-6 membered heterocycle, wherein said aryl, cycloalkyl and heterocycle is optionally substituted with R²⁴;
R¹² is independently selected from H and halogen;
R¹³ is independently selected from H, F, Cl, methyl, ethyl, isopropyl, and OCH₃;
R¹⁴ is independently selected from H, F, and Cl;
R¹⁵ is independently selected from pyrazolyl, aminopyrazolyl, pyrrolidinyl, triazolonyl, aminothiadiazolyl, oxazolyl, thiazolyl, pyridinyl, pyrimidinyl, and 2-pyridonyl;
R¹⁶ is independently selected from H, CN, OR^{b}, halogen, NR^{a}R^{a}, and C₁₋₃ alkyl substituted with 0-5 R^{e};
R¹⁷ is independently selected from H, methyl and halogen;
R¹⁸ is independently selected from H, methyl and halogen;
R¹⁹ is independently selected from C₁₋₄ alkyl, OC₁₋₄ alkyl, SC₁₋₄ alkyl, and C₃₋₆ cycloalkyl;
R²⁰ is independently selected from H, halogen, CN, and C₁₋₄alkyl;
R²¹ is independently selected from C₁₋₄ alkyl, OC₁₋₄ alkyl, SC₁₋₄ alkyl, and C₃₋₆ cycloalkyl;
R²² is independently selected from C₁₋₄alkyl and NR^{a}R^{a};
R²⁴, at each occurrence, is independently selected from H, F, Cl, Br, C(═O)NR^{a}R^{a}, C(═O)OR^{b}, —(CH₂)ₙ—OR^{b}, —(CH₂)ₙ—NR^{a}R^{a}, C₁₋₆ alkyl substituted with 0-5 R^{e}, aryl substituted with 0-5 R^{e}, —(CH₂)ₙ—C₃₋₆ cycloalkyl substituted with 0-5 R^{e}, —(CH₁)ₙ—O-4 to 10-membered heterocyclyl substituted with 0-5 Rₑ.
R^{a}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ-C₃₋₁₀carbocyclyl substituted with 0-5 R^{e}, and —(CH₂)ₙ-heterocyclyl substituted with 0-5 R^{e}; or R^{a} and R^{a} together with the nitrogen atom to which they are both attached form a heterocyclic ring substituted with 0-5 R^{e};
R^{b}, at each occurrence, is independently selected from H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, and —(CH₂)ₙ-heterocyclyl substituted with 0-5 R^{e};
R^{c}, at each occurrence, is independently selected from C₁₋₆ alkyl substituted with 0-5 R^{e}, C₂₋₆alkenyl substituted with 0-5 R^{e}, C₂₋₆alkynyl substituted with 0-5 R^{e}, C₃₋₆carbocyclyl, and heterocyclyl;
R^{d}, at each occurrence, is independently selected from H and C₁₋₄alkyl substituted with 0-5 R^{e};
R^{e}, at each occurrence, is independently selected from F, Cl, Br, CN, NO₂, ═O, C₁₋₆alkyl substituted with 0-5 R^{e}, C₂₋₆ alkenyl, C₂₋₆ alkynyl, —(CH₂)ₙ-C₃₋₆ cycloalkyl, —(CH₂)₁-aryl, —(CH₂)ₙ-heterocyclyl, CO₂H, —(CH₂)ₙOR^{f}, SR^{f}, and —(CH₂)ₙNR^{f}R^{f};
R^{f}, at each occurrence, is independently selected from H, C₁₋₅ alkyl optionally substituted with F, Cl, Br, C₃₋₆ cycloalkyl, and phenyl, or R^{f} and R^{f} together with the nitrogen atom to which they are both attached form a heterocyclic ring optionally substituted with C₁₋₄alkyl;
n, at each occurrence, is an integer independently selected from 0, 1, 2, and 4; and
p, at each occurrence, is an integer independently selected from 0, 1, and 2.

13. The compound according to claim 1, or a stereoisomer, a stereoisomer, an enantiomer, a tautomer, a pharmaceutically acceptable salt thereof, wherein: 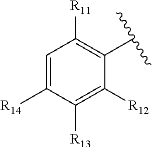
of (P5), (P6), (P7), P(10) and (P11) is independently selected from

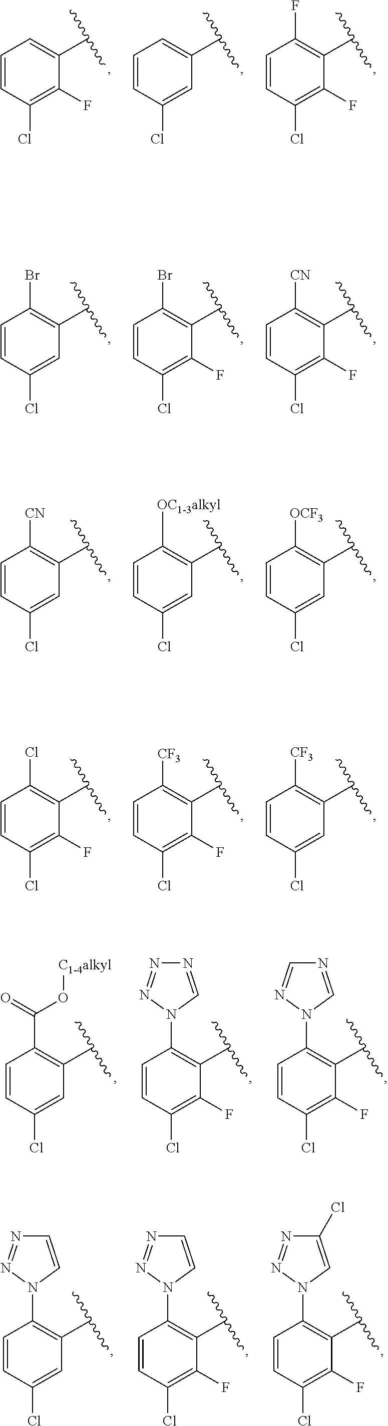

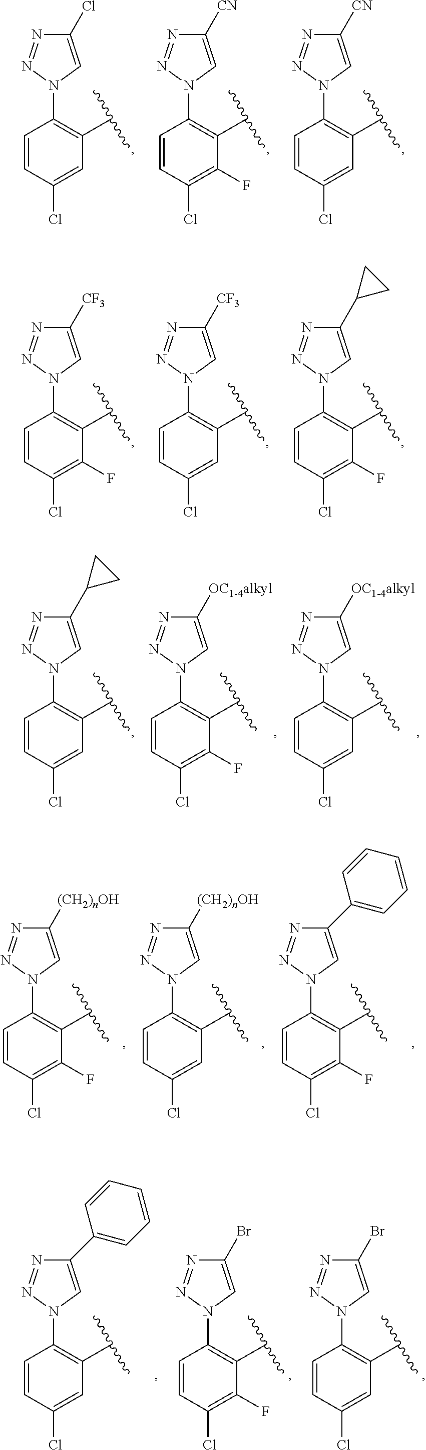

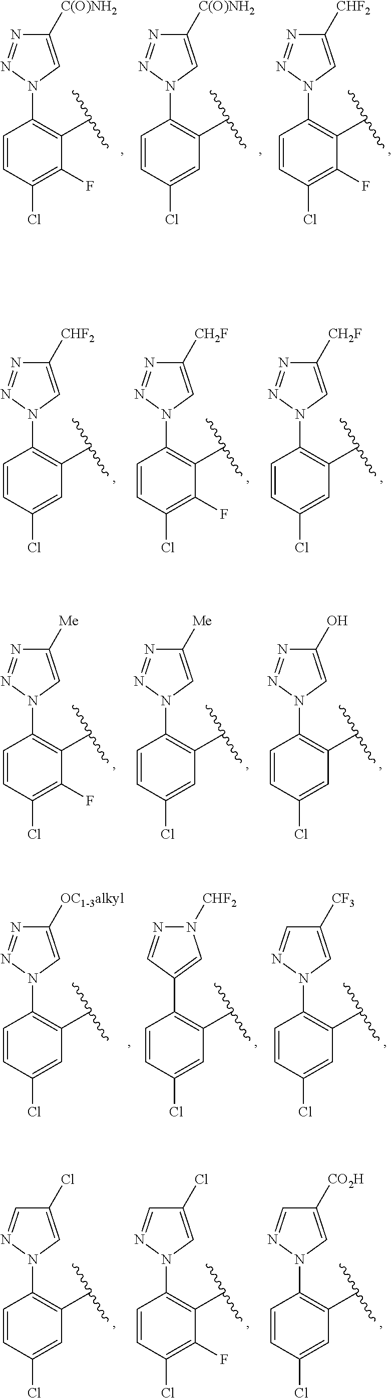

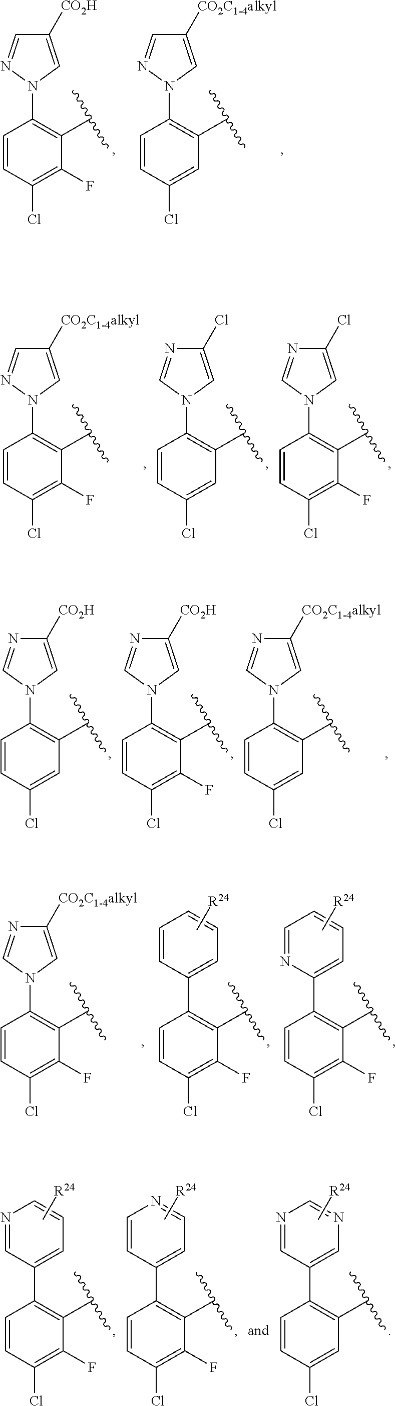

14. A pharmaceutical composition comprising one or more compounds according to claim 1 and a pharmaceutically acceptable carrier or diluent.

15. A method for the treatment and/or prophylaxis of a thromboembolic disorder, comprising: administering to a patient in need thereof a therapeutically effective amount of a compound of claim 1, or a stereoisomer, a tautomer, or a pharmaceutically acceptable salt thereof, wherein the thromboembolic disorder is selected from arterial cardiovascular thromboembolic disorders, venous cardiovascular thromboembolic disorders, and thromboembolic disorders in the chambers of the heart or in the peripheral circulation.

16. A method according to claim 15, wherein the thromboembolic disorder is selected from unstable angina, an acute coronary syndrome, atrial fibrillation, myocardial infarction, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from medical implants, devices, or procedures in which blood is exposed to an artificial surface that promotes thrombosis.
